(19) United States
(12) Patent Application Publication
(10) Pub. No.: US 2023/0212590 A1
(43) Pub. Date: Jul. 6, 2023

(54) NUCLEIC ACID CONSTRUCTS FOR PROTEIN MANUFACTURE

(71) Applicant: CATALENT PHARMA SOLUTIONS, LLC, Somerset, NJ (US)

(72) Inventors: Gregory T. Bleck, Cross Plains, WI (US); Rachel H. Kravitz, Madison, WI (US); Chad A. Hall, Black Earth, WI (US)

(21) Appl. No.: 18/007,568

(22) PCT Filed: Jun. 2, 2021
(86) PCT No.: PCT/US2021/035404

§ 371 Date: Dec. 1, 2022

Related U.S. Application Data
(60) Provisional application No. 63/033,514, filed on Jun. 2, 2020.
(60) Provisional application No. 63/033,516, filed on Jun. 2, 2020.

(51) Int. Cl. C12N 15/64; C12N 15/90

(52) U.S. Cl. CPC C12N 15/64; C12N 15/90

(57) ABSTRACT

The present invention relates to nucleic acid constructs and their use to develop host cell lines for production of a protein of interest, and in particular to nucleic acid constructs which allow for improved selection to develop high-producing cell lines.

### CROSS-REFERENCE TO RELATED APPLICATIONS

This application claims the benefit of U.S. Prov. Appl. 63/033,514, filed Jun. 2, 2020, the entire contents of which are incorporated herein by reference.

This application also claims the benefit of U.S. Prov. Appl. 63/033,516, filed Jun. 2, 2020, the entire contents of which are incorporated herein by reference.

### FIELD OF THE INVENTION

The present invention relates to nucleic acid constructs and their use to develop host cell lines for production of a protein of interest, and in particular to nucleic acid constructs which allow for improved selection to develop high-producing cell lines.

### BACKGROUND OF THE INVENTION

Therapeutic protein drugs are an important class of medicines serving patients most in need of novel therapies. Recently approved recombinant protein therapeutics have been developed to treat a wide variety of clinical indications, including cancers, autoimmunity/inflammation, exposure to infectious agents, and genetic disorders. The latest advances in protein-engineering technologies have allowed drug developers and manufacturers to fine-tune and exploit desirable functional characteristics of proteins of interest while maintaining (and in some cases enhancing) product safety or efficacy or both.

The manufacturing and production of therapeutic proteins are highly complex processes. For example, a typical protein drug may include in excess of 5,000 critical process steps, many times greater than the number required for manufacturing a small-molecule drug.

Similarly, protein therapeutics, which include monoclonal antibodies as well as large or fusion proteins, can be orders-of-magnitude larger in size than small-molecule drugs, having molecular weights exceeding 100 kDa. In addition, protein therapeutics exhibit complex secondary and tertiary structures that must be maintained. Protein therapeutics cannot be completely synthesized by chemical processes and have to be manufactured in living cells or organisms; consequently, the choices of the cell line, species origin, and culture conditions all affect the final product characteristics. Moreover, most biologically active proteins require post-translational modifications that can be compromised when heterologous expression systems are used. Additionally, as the products are synthesized by cells or organisms, complex purification processes are involved. Furthermore, viral clearance processes such as removal of virus particles by using filters or resins, as well as inactivation steps by using low pH or detergents, are implemented to prevent the serious safety issue of viral contamination of protein drug substances. Given the complexity of therapeutic proteins with respect to their large molecular size, post-translational modifications, and the variety of biological materials involved in their manufacturing process, the ability to enhance particular functional attributes while maintaining product safety and efficacy achieved through protein-engineering strategies is highly desirable.

While the integration of novel strategies and approaches to modify protein drug products is not a trivial matter, the potential therapeutic advantages have driven the increased use of such strategies during drug development. A number of protein-engineering platform technologies are currently in use to increase the circulating half-life, targeting, and functionality of novel therapeutic protein drugs as well as to increase production yield and product purity. For example, protein conjugation and derivatization approaches, including Fc-fusion, albumin-fusion, and PEGylation, are currently being used to extend a drug's circulating half-life.

The production of protein pharmaceutical (biologics) is expensive and time consuming. What is needed in the art are more efficient tools and processes for producing this important class of drugs.

### SUMMARY OF THE INVENTION

The present invention relates to nucleic acid constructs and their use to develop host cell lines for production of a protein of interest, and in particular to nucleic acid constructs which allow for improved selection to develop high-producing cell lines.

In some preferred embodiments, the present invention provides nucleic acid constructs for expression of a protein of interest comprising the following elements in operable association in 5′ to 3′ order: optionally, a first promoter sequence; a selectable marker sequence; a second promoter sequence; a nucleic acid sequence encoding a first protein of interest that is operably linked to the second promoter sequence; and a poly A signal sequence; the nucleic acid construct further comprising at least one insertion element at a position or positions selected from the group consisting of 5′ to the optional first promoter or selectable marker sequence, 3′ to the poly A signal sequence, between the optional first promoter and the poly A signal sequence, between the selectable marker and the second promoter sequence, and both 5′ to the optional first promoter sequence or the selectable marker sequence and 3′ to the poly A signal sequence. In some embodiments, nucleic acid constructs comprise the first promoter sequence. In some preferred embodiments, the construct does not comprise a poly A signal sequence between the selectable marker and the second promoter. In some preferred embodiments, the selectable marker is adjacent to the second promoter. In some preferred embodiments, the second promoter is adjacent to the nucleic acid sequence encoding the first protein of interest. In some preferred embodiments, the nucleic acid construct comprises a non-coding region between the first promoter and the selectable marker. In some preferred embodiments, the non-coding region comprises multiple potential Kozak sequences and/or ATG translation start sites. In some preferred embodiments, the nucleic acid construct comprises an extending packaging region (EPR) between the first promoter and the selectable marker. In some preferred embodiments, the EPR comprises multiple potential Kozak sequences and/or ATG translation start sites.

In some preferred embodiments, the first promoter sequence is selected from the group consisting of SIN-LTR, SV40, E. coli lac, E. coli trp, phage lambda PL, phage lambda PR, T3, T7, cytomegalovirus (CMV) immediate early, herpes simplex virus (HSV) thymidine kinase, alpha-lactalbumin, human elongation factor 1 alpha (hEF1alpha), and mouse metallothionein-I promoter sequences. In some preferred embodiments, the first promoter sequence is not a retroviral LTR promoter.

In some preferred embodiments, the selectable marker sequence is an amplifiable selectable marker sequence selected from the group consisting of the Glutamine Synthase (GS) sequence and the Dihydrofolate Reductase (DHFR) sequence. In some preferred embodiments, the selectable marker sequence is an antibiotic resistance marker sequence selected from the group consisting of neomycin resistance gene (neo), hygromycin B phosphotransferase gene and puromycin N-acetyl transferase gene sequences.

In some preferred embodiments, the second promoter sequence is selected from the group consisting of SV40, E. coli lac, E. coli trp, phage lambda PL, phage lambda PR, T3, T7, cytomegalovirus (CMV) immediate early, herpes simplex virus (HSV) thymidine kinase, alpha-lactalbumin, human elongation factor 1 alpha (hEF1alpha), and mouse metallothionein-I promoter sequences.

In some preferred embodiments, the nucleic acid sequence encoding a protein of interest encodes a protein selected from the group consisting of heavy and light chain immunoglobulin sequences.

In some preferred embodiments, the insertion element is selected from the group consisting of a transposon insertion element, a recombinase insertion element, and a HDR insertion element. In some preferred embodiments, the transposon insertion element is an inverted terminal repeat. In some preferred embodiments, the construct comprises two inverted terminal repeats positioned 5′ to the first promoter and 3′ to the poly A signal sequence. In some preferred embodiments, the recombinase insertion element is an attachment site (att). In some preferred embodiments, the attachment site (aft) is attB. In some preferred embodiments, the HDR insertion element comprises an AAVS1 safe harbor locus sequence. In some preferred embodiments, the HDR insertion element is a nucleic acid sequence homologous to a target site in a chromosome. In some preferred embodiments, the nucleic acid sequence homologous to a target site in a chromosome is from about 30 to 1000 bases in length. In some preferred embodiments, the construct comprises two nucleic acid sequences homologous to a target site in a chromosome positioned 5′ to the first promoter and 3′ to the poly A signal sequence. In some preferred embodiments, the recombinase insertion element is a Flp Recombination Target (FRT) site. In some preferred embodiments, the recombinase insertion element is a LoxP sequence.

In some preferred embodiments, the constructs further comprise an RNA export element. In some preferred embodiments, the RNA export element is located 3′ or 5′ to the nucleic acid sequence encoding the protein of interest. In some preferred embodiments, the RNA export element is a pre-mRNA processing enhancer (PPE). In some preferred embodiments, the RNA export element is a posttranscriptional regulatory element (PRE). In some preferred embodiments, the PRE RNA export element is a Woodchuck hepatitis virus post-transcriptional regulatory element (WPRE).

In some preferred embodiments, the constructs further comprise a signal peptide sequence operably linked to the first protein of interest. In some preferred embodiments, the signal peptide sequence is selected from the group consisting of tissue plasminogen activator, human growth hormone, lactoferrin, alpha-casein and alpha-lactalbumin signal peptide sequences.

In some preferred embodiments, the constructs further comprise a protein purification marker sequence. In some preferred embodiments, the protein purification marker sequence is a hexahistidine tag or a hemagglutinin (HA) tag.

In some preferred embodiments, the constructs further comprise an Internal Ribosome Entry Site (IRES) sequence and a second nucleic acid sequence encoding at least a second protein of interest (e.g., including third, fourth, fifth, etc. protein of interest) positioned 3′ to the nucleic acid sequence encoding the first protein of interest. In some preferred embodiments, the IRES sequence is selected from the group consisting of foot and mouth disease virus (FDV), encephalomyocarditis virus and poliovirus IRES sequences.

In some preferred embodiments, the nucleic acid construct further comprises a third promoter operably linked to a second nucleic acid sequence encoding a second protein of interest positioned 3′ to the nucleic acid sequence encoding the first protein of interest. In some preferred embodiments, the third promoter sequence is selected from the group consisting of SV40, E. coli lac, E. coli trp, phage lambda PL, phage lambda PR, T3, T7, cytomegalovirus (CMV) immediate early, herpes simplex virus (HSV) thymidine kinase, alpha-lactalbumin, human elongation factor 1 alpha (hEF1alpha), and mouse metallothionein-I promoter sequences. In some preferred embodiments, the constructs further comprise an RNA export element in operable association with the second nucleic acid sequence encoding a second protein of interest. In some preferred embodiments, the constructs further comprise a poly A signal sequence in operable association with the second nucleic acid sequence encoding a second protein of interest.

In some preferred embodiments, the first protein of interest is one of an antibody heavy and light chain and the second protein of interest is the other of an antibody heavy and light chain.

In some preferred embodiments, the nucleic acid construct further comprises an intron operably linked to a second nucleic acid sequence encoding a second protein of interest positioned 3′ to the nucleic acid sequence encoding the first protein of interest. In some preferred embodiments, the constructs further comprise an RNA export element in operable association with the second nucleic acid sequence encoding a second protein of interest. In some preferred embodiments, the constructs further comprise a poly A signal sequence in operable association with the second nucleic acid sequence encoding a second protein of interest. In some preferred embodiments, the first protein of interest is one of an antibody heavy and light chain and the second protein of interest is the other of an antibody heavy and light chain.

In some preferred embodiments, the present invention provides a vector comprising a nucleic acid construct as described above. In some preferred embodiments, the vector is a plasmid.

In some preferred embodiments, the present invention provides a host cell comprising a nucleic acid construct as described above or a vector as described above. In some preferred embodiments, the host cell is selected from the group consisting of Chinese Hamster Ovary (CHO) cells, HEK 293 cells, CAP cells, bovine mammary epithelial cells, monkey kidney CV1 line transformed by SV40, baby hamster kidney cells, mouse sertoli cells, monkey kidney cells, African green monkey kidney cells, human cervical carcinoma cells, canine kidney cells, buffalo rat liver cells, human lung cells, human liver cells, mouse mammary tumor, TRI cells, MRC 5 cells, FS4 cells, rat fibroblasts, MDBK cells and human hepatoma line cells. In some preferred embodiments, the host cell is selected from the group consisting of a Chinese Hamster Ovary (CHO) cells, a HEK 293 cells and a CAP cells. In some preferred embodiments, the host cell line is a GS knockout cell line. In some preferred embodiments, the host cell line is a DHFR knockout cell line. In some preferred embodiments, the host cell comprises from about 1 to 1000 copies of the nucleic acid construct. In some preferred embodiments, the host cell comprises from about 10 to 200 copies of the nucleic acid construct. In some preferred embodiments, the host cell comprises from about 10 to 100 copies of the nucleic acid construct. In some preferred embodiments, the host cell comprises from about 20 to 100 copies of the nucleic acid construct. In some preferred embodiments, the host cell comprises from 50 to 500 copies of the nucleic acid construct. In some preferred embodiments, the host cell comprises from 50 to 250 copies of the nucleic acid construct.

In some preferred embodiments, the host cell further comprises at least a second nucleic acid construct that encodes and allows for expression of a second protein of interest. In some preferred embodiments, the second nucleic acid construct does not include a selectable marker. In some preferred embodiments, the second nucleic acid construct includes a selectable marker that is different from the selectable marker in the first nucleic acid construct. In some preferred embodiments, the first protein of interest in the first nucleic acid construct is one of an immunoglobulin heavy or light chain and the second protein in the second nucleic acid construct is the other of an immunoglobulin heavy or light chain. In some preferred embodiments, the first protein of interest is an immunoglobulin heavy chain and the second protein of interest is an immunoglobulin light chain. In some preferred embodiments, the host cell comprises from about 1 to 1000 copies of the second nucleic acid construct. In some preferred embodiments, the host cell comprises from about 10 to 200 copies of the second nucleic acid construct. In some preferred embodiments, the host cell comprises from about 10 to 100 copies of the second nucleic acid construct. In some preferred embodiments, the host cell comprises from about 20 to 100 copies of the second nucleic acid construct. In some preferred embodiments, the host cell comprises from 50 to 500 copies of the second nucleic acid construct. In some preferred embodiments, the host cell comprises from 50 to 250 copies of the second nucleic acid construct.

In some preferred embodiments, the present invention provides a host cell culture comprising a population of host cells as described above.

In some preferred embodiments, the present invention provides processes for producing a protein of interest comprising culturing host cells as described above under conditions such that the protein(s) of interest is expressed and purifying the protein(s) of interest from the host cell culture. In some preferred embodiments, the host cells grown in a medium comprising an inhibitor of the selectable marker. In some preferred embodiments, the selectable marker is GS and the inhibitor is phosphinothricin or methionine sulphoximine (Msx). In some preferred embodiments, the selectable marker is DHFR and the inhibitor is methotrexate.

In some preferred embodiments, the present invention provides a vector comprising the nucleic acid construct as described above. In some preferred embodiments, the vector is selected from the group consisting of a plasmid vector, a retroviral vector, a lentiviral vector, an AAV vector, and a transposon vector.

In some preferred embodiments, the present invention provides a system comprising: a first nucleic acid construct as described above; and a second nucleic acid construct encoding an enzyme. In some preferred embodiments, the constructs are provided on different vectors. In some preferred embodiments, the constructs are provided on the same vectors. In some preferred embodiments, the enzyme is selected from the group consisting of a transposase, an integrase, a recombinase, a nuclease and a nickase. In some preferred embodiments, the nuclease is a Cas nuclease. In some preferred embodiments, the nickase is a Cas nickase. In some preferred embodiments, the systems further comprise one or more RNA guide sequences. In some preferred embodiments, the enzyme facilitates insertion of the nucleic acid construct or a portion thereof into the genome of a host cell.

In some preferred embodiments, the systems further comprise at least a third nucleic acid construct as described above, the third nucleic acid construct encoding a protein of interest that is different from the protein of interest in the first nucleic acid construct. In some preferred embodiments, the third nucleic acid construct is provided in a separate vector. In some preferred embodiments, the third nucleic acid construct is provided in the same vector as the first and second nucleic acid constructs.

In some preferred embodiments, the present invention provides a system comprising at least first and second nucleic acid constructs as described above; wherein the first and second nucleic acid constructs each encode a different protein of interest. In some preferred embodiments, the first and second nucleic acid constructs are provided in separate vectors. In some preferred embodiments, the first and second nucleic acid constructs are provided in the same vector. In some preferred embodiments, the systems further comprise a third nucleic acid construct encoding an enzyme. In some preferred embodiments, the enzyme is selected from the group consisting of a transposase, an integrase, a recombinase, a nuclease and a nickase. In some preferred embodiments, the nuclease is a Cas nuclease. In some preferred embodiments, the nickase is a Cas nickase. In some preferred embodiments, the systems further comprise one or more RNA guide sequences. In some preferred embodiments, the enzyme facilitates insertion of the nucleic acid construct or a portion thereof into the genome of a host cell. In some preferred embodiments, the third nucleic acid construct is provided in a separate vector. In some preferred embodiments, the third nucleic acid construct is provided in the same vector as the first and second nucleic acid constructs.

In some preferred embodiments, the present invention provides processes for producing a protein of interest comprising: introducing a nucleic acid construct, vector, or a system as described above into a host cell under conditions such that the nucleic acid construct is incorporated into the genome of the host cell; developing a host cell line that expresses the protein of interest; culturing host cells from the host cell line under conditions such that the protein of interested is produced by the host cells; and purifying the protein of interest from the host cell culture.

In some preferred embodiments, the host cell is selected from the group consisting of Chinese Hamster Ovary (CHO) cells, HEK 293 cells, CAP cells, bovine mammary epithelial cells, monkey kidney CV1 line transformed by SV40, baby hamster kidney cells, mouse sertoli cells, monkey kidney cells, African green monkey kidney cells, human cervical carcinoma cells, canine kidney cells, buffalo rat liver cells, human lung cells, human liver cells, mouse mammary tumor, TRI cells, MRC 5 cells, FS4 cells, rat fibroblasts, MDBK cells and human hepatoma line cells. In some preferred embodiments, the host cell is selected from the group consisting of a Chinese Hamster Ovary (CHO) cells, a HEK 293 cells and a CAP cells. In some preferred embodiments, the host cell line is a GS knockout cell line. In some preferred embodiments, the host cell line is a DHFR knockout cell line. In some preferred embodiments, the host cells are grown in a medium comprising an inhibitor of the selectable marker. In some preferred embodiments, the selectable marker is GS and the inhibitor is phosphinothricin or methionine sulphoximine (Msx). In some preferred embodiments, the selectable marker is DHFR and the inhibitor is methotrexate. In some preferred embodiments, culturing host cells from the host cell line under conditions such that the protein of interested is produced by the host cells further comprises culturing in a system selected from the group consisting of petri dishes, well plates, roller bottles, bioreactors, perfusion systems and fed batch cultures.

### DESCRIPTION OF THE FIGURES

Abbreviations used in figures:

AmpR=bacterial ampicillin resistance gene

attB=Bacterial Attachment Site

attP=Phage Attachment Site

attR=Recombined Upstream Attachment Site

Backbone=Plasmid Backbone

CDS=Coding Sequence

EPR=MMLV Extended Packaging Region

GCI=Gene Copy Index

GS=Glutamine Synthetase

H or HC═Heavy Chain

hCMV=Human Cytomegalovirus immediate-early Promoter

I=intron

L or LC=Light Chain

MoMuSV 5′LTR=Moloney Murine Sarcoma Virus 5′ Long Terminal Repeat

Neo=Neomycin resistance genePA or PolyA=Polyadenylation signal

ProV SIN-LTR=Proviral Self-Inactivating Long Terminal Repeat

sCMV=Simian Cytomegalovirus immediate-early Promoter

SDS-PAGE=Sodium Dodecyl Sulphate-Polyacrylamide Gel Electrophoresis

SIN-3′LTR=Self-Inactivation 3′ Long Terminal Repeat

SV40=Simian Virus 40

TK=Thymidine Kinase

UTR=Untranslated Region

W or WPRE=Woodchuck Post-transcriptional Regulatory Element

FIG. 1. Nucleic acid construct design for certain embodiments of the invention

FIG. 2. Graph of cell survival curves after transfection and selection in the absence of glutamine. Averages from duplicate transfections are shown.

FIG. 3. Chart depicting productivity and copy number analysis of pooled cell lines made using different plasmids. Averages from duplicate transfections are shown.

FIG. 4. Graph of cell survival curves after transfection and selection in the absence of glutamine. Averages from duplicate transfections are shown.

FIG. 5. PhiC31 Integrase Expression Plasmid Map.

FIG. 6. PhiC31 Integrase Expression Plasmid Sequence.

FIG. 7. Dock Plasmid Map.

FIG. 8. Dock Plasmid Sequence.

FIG. 9. Dock-WPRE Plasmid Map.

FIG. 10. Dock-WPRE Plasmid Sequence.

FIG. 11. Transgene-Promoter-Anyway Plasmid Map. In this plasmid, the expression of GS is driven by the weak, Moloney Murine Sarcoma Virus 5′ proviral self-inactivation Long Terminal Repeat.

FIG. 12. Transgene-Promoter-Anyway Plasmid Sequence. In plasmid and all subsequent Transgene plasmids, there is no promoter to drive GS expression in the Transgene plasmid.

FIG. 13. Transgene-Anyway Plasmid Map

FIG. 14. Transgene-Anyway Plasmid Sequence

FIG. 15. Transgene-MCS Plasmid Map

FIG. 16. Transgene-MCS Plasmid Sequence

FIG. 17. Transgene-MCS-WPRE-Intron-MCS Plasmid Map

FIG. 18. Transgene-MCS-WPRE-Intron-MCS Plasmid Sequence

FIG. 19. Transgene-MCS-WPRE-MCS-WPRE Plasmid Map

FIG. 20. Transgene-MCS-WPRE-MCS-WPRE Plasmid Sequence

FIG. 21. Transgene-Yourway-HWIL Plasmid Map

FIG. 22. Transgene-Yourway-HWIL Plasmid Sequence

FIG. 23. Transgene-Yourway-LWIH Plasmid Map

FIG. 24. Transgene-Yourway-LWIH Plasmid Sequence

FIG. 25. Transgene-Yourway-HWLW Plasmid Map

FIG. 26. Transgene-Yourway-HWLW Plasmid Sequence

FIG. 27. Transgene-Yourway-LWHW Plasmid Map

FIG. 28. Transgene-Yourway-LWHW Plasmid Sequence

FIG. 29. Graph of unselected attR gene copy index from Dock cell pools containing approximately 36 Docks per cell, on average, transfected with the Transgene-Promoter-Anyway plasmid at the indicated ratios.

FIG. 30. Graph of percent viable cells over time of selection from select pools in FIG. 29.

FIG. 31. Chart of attR gene copy indexes and copy numbers of all pools from FIG. 30.

FIG. 32. Graph of percent viable cells over time of selection from Dock cell pools containing approximately 135 Docks per cell, on average, transfected with the promoterless Transgene-Anyway plasmid and Integrase plasmid at the indicated ratios. The average of duplicate pools is shown.

FIG. 33. Chart of attR gene copy indexes of pools from FIG. 32 after selection. The average of duplicate pools is shown.

FIG. 34. Graph of percent viable cells over time of selection from Dock clone cells containing approximately 181 copies of Dock per cell transfected with the Transgene-Yourway-LWHW plasmid and Integrase plasmid at the indicated ratios. The average of duplicate pools is shown.

FIG. 35. Chart of attR gene copy indexes of pools from FIG. 34 after selection. The average of duplicate pools is shown.

FIG. 36. Chart of attR (filled dock) and attP (empty dock) gene copy indexes, % filled Docks, and final titer from fed-batch productivity from clones made from Dock pools containing approximately 135 copies of Dock per cell transfected with the Transgene-Anyway plasmid and Integrase plasmid.

FIG. 37. Graph of Excell Fed-batch productivity titer versus attR gene copy indexes for all 25 clones in FIG. 36.

FIG. 38. Graph of percent viable cells over time of selection of Dock clone cells containing approximately 181 copies of Dock per cell transfected with the Transgene-Yourway-LWHW, Yourway-HWLW, Yourway-HWIL, Yourway-LWIH, or Anyway plasmids (individually) and Integrase plasmid. The average of duplicate pools is shown.

FIG. 39. Chart of attR gene copy indexes and final titer from fed-batch productivity of clones made from Dock pools from FIG. 38. The average of duplicate pools is shown.

FIG. 40. SDS-PAGE analysis of Transgene-Yourway and Transgene-Anyway products run under both nonreducing (left) and reducing conditions (right).

FIG. 41. Graph of final titer over 40 generations from fed-batch productivity using two different media/feeding strategies of 3 pools expressing Anyway.

### DEFINITIONS

To facilitate understanding of the invention, a number of terms are defined below.

As used herein, the term “host cell” refers to any eukaryotic cell (e.g., mammalian cells, avian cells, amphibian cells, plant cells, fish cells, and insect cells), whether located in vitro or in vivo.

As used herein, the term “cell culture” refers to any in vitro culture of cells. Included within this term are continuous cell lines (e.g., with an immortal phenotype), primary cell cultures, finite cell lines (e.g., non-transformed cells), and any other cell population maintained in vitro, including oocytes and embryos.

As used herein, the term “vector” refers to any genetic element, such as a plasmid, phage, transposon, cosmid, chromosome, virus, virion, etc., which is capable of replication when associated with the proper control elements and which can transfer gene sequences between cells. Thus, the term includes cloning and expression vehicles, as well as viral vectors.

As used herein, the term “genome” refers to the genetic material (e.g., chromosomes) of an organism.

The term “nucleotide sequence of interest” refers to any nucleotide sequence (e.g., RNA or DNA), the manipulation of which may be deemed desirable for any reason (e.g., treat disease, confer improved qualities, expression of a protein of interest in a host cell, expression of a ribozyme, etc.), by one of ordinary skill in the art. Such nucleotide sequences include, but are not limited to, coding sequences of structural genes (e.g., reporter genes, selection marker genes, oncogenes, drug resistance genes, growth factors, etc.), and non-coding regulatory sequences which do not encode an mRNA or protein product (e.g., promoter sequence, polyadenylation sequence, termination sequence, enhancer sequence, etc.).

As used herein, the term “protein of interest” refers to a protein encoded by a nucleic acid of interest.

As used herein, the terms “nucleic acid molecule encoding,” “DNA sequence encoding,” “DNA encoding,” “RNA sequence encoding,” and “RNA encoding” refer to the order or sequence of deoxyribonucleotides or ribonucleotides along a strand of deoxyribonucleic acid or ribonucleic acid. The order of these deoxyribonucleotides or ribonucleotides determines the order of amino acids along the polypeptide (protein) chain. The DNA or RNA sequence thus codes for the amino acid sequence.

The term “promoter,” “promoter element,” or “promoter sequence” as used herein, refers to a DNA sequence which when ligated to a nucleotide sequence of interest is capable of controlling the transcription of the nucleotide sequence of interest into mRNA. A promoter is typically, though not necessarily, located 5′ (i.e., upstream) of a nucleotide sequence of interest whose transcription into mRNA it controls, and provides a site for specific binding by RNA polymerase and other transcription factors for initiation of transcription.

Transcriptional control signals in eukaryotes comprise “promoter” and “enhancer” elements. Promoters and enhancers consist of short arrays of DNA sequences that interact specifically with cellular proteins involved in transcription (Maniatis et al., Science 236:1237) [1987]). Promoter and enhancer elements have been isolated from a variety of eukaryotic sources including genes in yeast, insect and mammalian cells, and viruses (analogous control elements, i.e., promoters, are also found in prokaryotes). The selection of a particular promoter and enhancer depends on what cell type is to be used to express the protein of interest. Some eukaryotic promoters and enhancers have a broad host range while others are functional in a limited subset of cell types (for review see, Voss et al., Trends Biochem. Sci., 11:287 [1986]; and Maniatis et al., supra). For example, the SV40 early gene enhancer is very active in a wide variety of cell types from many mammalian species and has been widely used for the expression of proteins in mammalian cells (Dijkema et al., EMBO J. 4:761 [1985]). Two other examples of promoter/enhancer elements active in a broad range of mammalian cell types are those from the human elongation factor 1a gene (Uetsuki et al., J. Biol. Chem., 264:5791 [1989]; Kim et al., Gene 91:217 [1990]; and Mizushima and Nagata, Nuc. Acids. Res., 18:5322 [1990]) and the long terminal repeats of the Rous sarcoma virus (Gorman et al., Proc. Natl. Acad. Sci. USA 79:6777 [1982]) and the human cytomegalovirus (Boshart et al., Cell 41:521 [1985]).

As used herein, the term “promoter/enhancer” denotes a segment of DNA which contains sequences capable of providing both promoter and enhancer functions (i.e., the functions provided by a promoter element and an enhancer element, see above for a discussion of these functions). For example, the long terminal repeats of retroviruses contain both promoter and enhancer functions. The enhancer/promoter may be “endogenous” or “exogenous” or “heterologous.” An “endogenous” enhancer/promoter is one that is naturally linked with a given gene in the genome. An “exogenous” or “heterologous” enhancer/promoter is one that is placed in juxtaposition to a gene by means of genetic manipulation (i.e., molecular biological techniques such as cloning and recombination) such that transcription of that gene is directed by the linked enhancer/promoter.

As used herein, the term “long terminal repeat” of “LTR” refers to transcriptional control elements located in or isolated from the U3 region 5′ and 3′ of a retroviral genome. As is known in the art, long terminal repeats may be used as control elements in retroviral vectors, or isolated from the retroviral genome and used to control expression from other types of vectors.

As used herein, the terms “complementary” or “complementarity” are used in reference to polynucleotides (i.e., a sequence of nucleotides) related by the base-pairing rules. For example, the sequence “5′-A-G-T-3′,” is complementary to the sequence “3′-T-C-A-5′.” Complementarity may be “partial,” in which only some of the nucleic acids' bases are matched according to the base pairing rules. Or, there may be “complete” or “total” complementarity between the nucleic acids. The degree of complementarity between nucleic acid strands has significant effects on the efficiency and strength of hybridization between nucleic acid strands. This is of particular importance in amplification reactions, as well as detection methods that depend upon binding between nucleic acids.

The terms “homology” and “percent identity” when used in relation to nucleic acids refers to a degree of complementarity. There may be partial homology (i.e., partial identity) or complete homology (i.e., complete identity). A partially complementary sequence is one that at least partially inhibits a completely complementary sequence from hybridizing to a target nucleic acid sequence and is referred to using the functional term “substantially homologous.” The inhibition of hybridization of the completely complementary sequence to the target sequence may be examined using a hybridization assay (Southern or Northern blot, solution hybridization and the like) under conditions of low stringency. A substantially homologous sequence or probe (i.e., an oligonucleotide which is capable of hybridizing to another oligonucleotide of interest) will compete for and inhibit the binding (i.e., the hybridization) of a completely homologous sequence to a target sequence under conditions of low stringency. This is not to say that conditions of low stringency are such that non-specific binding is permitted; low stringency conditions require that the binding of two sequences to one another be a specific (i.e., selective) interaction. The absence of non-specific binding may be tested by the use of a second target which lacks even a partial degree of complementarity (e.g., less than about 30% identity); in the absence of non-specific binding the probe will not hybridize to the second non-complementary target.

The terms “in operable combination,” “in operable order,” and “operably linked” as used herein refer to the linkage of nucleic acid sequences in such a manner that a nucleic acid molecule capable of directing the transcription of a given gene and/or the synthesis of a desired protein molecule is produced. The term also refers to the linkage of amino acid sequences in such a manner so that a functional protein is produced.

As used herein, the term “selectable marker” refers to a gene that encodes an enzymatic activity or other protein that confers the ability to grow in medium lacking what would otherwise be an essential nutrient; in addition, a selectable marker may confer resistance to an antibiotic or drug upon the cell in which the selectable marker is expressed.

As used herein, the term “retrovirus” refers to a retroviral particle which is capable of entering a cell (i.e., the particle contains a membrane-associated protein such as an envelope protein or a viral G glycoprotein which can bind to the host cell surface and facilitate entry of the viral particle into the cytoplasm of the host cell) and integrating the retroviral genome (as a double-stranded provirus) into the genome of the host cell. The term “retrovirus” encompasses Oncovirinae (e.g., Moloney murine leukemia virus (MoMLV), Moloney murine sarcoma virus (MoMSV), and Mouse mammary tumor virus (MMTV), Spumavirinae, amd Lentivirinae (e.g., Human immunodeficiency virus, Simian immunodeficiency virus, Equine infection anemia virus, and Caprine arthritis-encephalitis virus; See, e.g., U.S. Pat. Nos. 5,994,136 and 6,013,516, both of which are incorporated herein by reference).

As used herein, the term “retroviral vector” refers to a retrovirus that has been modified to express a gene of interest. Retroviral vectors can be used to transfer genes efficiently into host cells by exploiting the viral infectious process. Foreign or heterologous genes cloned (i.e., inserted using molecular biological techniques) into the retroviral genome can be delivered efficiently to host cells that are susceptible to infection by the retrovirus. Through well-known genetic manipulations, the replicative capacity of the retroviral genome can be destroyed. The resulting replication-defective vectors can be used to introduce new genetic material to a cell but they are unable to replicate. A helper virus or packaging cell line can be used to permit vector particle assembly and egress from the cell. Such retroviral vectors comprise a replication-deficient retroviral genome containing a nucleic acid sequence encoding at least one gene of interest (i.e., a polycistronic nucleic acid sequence can encode more than one gene of interest), a 5′ retroviral long terminal repeat (5′ LTR); and a 3′ retroviral long terminal repeat (3′ LTR).

As used herein, the term “lentivirus vector” refers to retroviral vectors derived from the Lentiviridae family (e.g., human immunodeficiency virus, simian immunodeficiency virus, equine infectious anemia virus, and caprine arthritis-encephalitis virus) that are capable of integrating into non-dividing cells (See, e.g., U.S. Pat. Nos. 5,994,136 and 6,013,516, both of which are incorporated herein by reference).

As used herein, the term “transposon” refers to transposable elements (e.g., Tn5, Tn7, and Tn10) that can move or transpose from one position to another in a genome. In general, the transposition is controlled by a transposase. The term “transposon vector,” as used herein, refers to a vector encoding a nucleic acid of interest flanked by the terminal ends of transposon. Examples of transposon vectors include, but are not limited to, those described in U.S. Pat. Nos. 6,027,722; 5,958,775; 5,968,785; 5,965,443; and 5,719,055, all of which are incorporated herein by reference.

As used herein, the term “adeno-associated virus (AAV) vector” refers to a vector derived from an adeno-associated virus serotype, including without limitation, AAV-1, AAV-2, AAV-3, AAV-4, AAV-5, AAVX7, etc. AAV vectors can have one or more of the AAV wild-type genes deleted in whole or part, preferably the rep and/or cap genes, but retain functional flanking ITR sequences.

AAV vectors can be constructed using recombinant techniques that are known in the art to include one or more heterologous nucleotide sequences flanked on both ends (5′ and 3′) with functional AAV ITRs. In the practice of the invention, an AAV vector can include at least one AAV ITR and a suitable promoter sequence positioned upstream of the heterologous nucleotide sequence and at least one AAV ITR positioned downstream of the heterologous sequence. A “recombinant AAV vector plasmid” refers to one type of recombinant AAV vector wherein the vector comprises a plasmid. As with AAV vectors in general, 5′ and 3′ ITRs flank the selected heterologous nucleotide sequence.

As used herein, the term “adenoviral vector” refers to a non-enveloped double-stranded DNA vector comprising an adenovirus backbone.

As used herein, the term “purified” refers to molecules, either nucleic or amino acid sequences, that are removed from their normal environment, isolated or separated. An “isolated nucleic acid sequence” is therefore a purified nucleic acid sequence. “Substantially purified” molecules are at least 60% free, preferably at least 75% free, and more preferably at least 90% free from other components with which they are normally associated.

### DETAILED DESCRIPTION OF THE INVENTION

The present invention relates to nucleic acid constructs and their use to develop host cell lines for production of a protein of interest, and in particular to nucleic acid constructs which allow for improved selection to develop high-producing cell lines.

In some preferred embodiments, the present invention provides nucleic acid constructs for use in expressing a protein or proteins of interest in a host cell. In some preferred embodiments, the nucleic acid constructs comprise the following elements in operable association, most preferably in 5′ to 3′ order:

first promoter sequence—selectable marker sequence—second promoter sequence—nucleic acid sequence encoding a first protein of interest—poly A signal sequence.

In some preferred embodiments, the constructs of the invention do not comprise a poly A signal sequence between the selectable marker sequence and second promoter sequence. The present invention is not limited to any particular mechanism of action. Indeed, an understanding of the mechanism of action is not necessary to practice the present invention. Nevertheless, constructs which lack a poly A signal sequence after the selectable marker have been found to provide for better selection and production of the protein of interest in host cell cultures. In still other preferred embodiments, the selectable marker is adjacent to the second promoter. In still other preferred embodiments, the second promoter is adjacent to the nucleic acid sequence encoding the first protein of interest. In this context, the term “adjacent” means that there is no intervening functional element or intron between the listed components.

The nucleic acid constructs may be utilized with many different vectors and vectors systems. Suitable vectors and vectors systems include, but are not limited to, viral gene insertion technologies such as retroviral, lentiviral and AAV systems as well as non-viral gene insertion technologies such as transposase, recombinase, integrase or CRISPR gene insertion. Specific examples of technologies/enzymes that can be used with nucleic acid constructs of the present invention include piggyback transposase systems, sleeping beauty transposase systems, Mos1 transposase systems, To12 transposase systems, Leapin transposase systems, Lambda recombinase systems, FLP/FRT systems, Cre/Lox systems, MMLV integrase systems, Rep 78 integrase systems and CRISPR systems which can include nucleases or nickases as well as guide sequences. In some preferred embodiments, the system is a nucleic acid integration system with the proviso that the system is not a retroviral or lentiviral systems utilizing a retroviral or lentiviral LTR.

In some embodiments, the constructs are useful in host cells comprise integrated docking sites as described in U.S. Prov. Appl. 63/033,516, the entire contents of which are incorporated here by reference. The integrated docking sites preferably comprise one or more insertion elements (which may be termed a “dock site insertion element.” The dock site insertion elements are preferably nucleic acid sequences that facilitate insertion of a nucleic acid sequence encoding a protein of interest at the dock site. Nucleic acid constructs that can be inserted into the dock sites in the host cells of the present invention are described in detail below.

For example, in some preferred embodiments, the recombinase dock site insertion element comprises an attachment site (att). In some particularly preferred embodiments, the attachment site is attP. These attachment sites are utilized by the PhiC31 integrase, which is a recombinase enzyme and which can be provided in the host cell via a vector in preferred embodiments. These dock sites serve as acceptors for integration of nucleic acid constructs comprising an attB attachment site. In other preferred embodiments, attR and attL attachment sites may be utilized.

In other preferred embodiments, the recombinase dock site insertion element comprises an Flp Recombination Target (FRT) site. These sites are utilized by the enzyme flippase, which is a recombinase enzyme and which can be provided in the host cell via a vector in preferred embodiments. These dock sites serve as acceptors for integration of nucleic acid constructs comprising at the FRT site.

In other preferred embodiments, the recombinase dock site insertion element comprises a LoxP site. These sites are utilized by the Cre recombinase which can be provided in the host cell via a vector in preferred embodiments. These dock sites serve as acceptors for integration of nucleic acid constructs comprising the LoxP site.

In other preferred embodiments, the insertion element is an HDR (homology directed repair) dock site insertion element. HDR dock site insertion elements are nucleic acid sequences that provide an area of homology (a “homology arm”) that base pair with corresponding homology arms on the nucleic acid construct that is inserted at the site. These systems are preferably used with endonucleases that introduce double stranded breaks at a targeted site or sites, preferably flanked by the homology arms. In some embodiments, the HDR dock site insertion element is an AAVS1 safe harbor locus. In these embodiments, the dock site is used utilized by the Rep 78 endonuclease (nickase) which may be introduced into the host cell via a vector. The Rep 78 protein nickase promotes site-specific integration of nucleic acid sequences bearing homology arms corresponding to the AAVS1 safe harbor locus.

In other preferred embodiments, the HDR dock site insertion element comprises one or more homology arms that are exogenous sequences of from 30 to 1000 base pairs in length. These dock sites are preferably used in conjunction with CRISPR gene editing systems. In some embodiments, the dock site further comprises one or more sequences that are homologous to guide RNA sequences. In these embodiments, the nucleic acid construct that is inserted at the dock site preferably comprises homology arms that are homologous to and base pair with the homology arms in the dock site. For utilization with CRISPR gene editing systems, a CRISPR gene editing system-compatible nuclease is introduced into the host cell. The CRISPR gene editing system-compatible nuclease may be a wild-type endonuclease that creates a double-stranded break at a position determined by the guide RNA (and within the docking site) or a mutated nuclease (i.e., a nickase) that creates a single stranded break at a staggered positions within the dock site defined by two guide RNAs. Suitable nucleases are described in detail below in the discussion of nucleic acid expression constructs.

In some preferred embodiments, the docking site may preferably comprise a suitable promoter so that a promoter trap scheme is utilized when suitable nucleic acid constructs are introduced at the docking site. Suitable promoters include, but are not limited to, SIN-LTR, SV40, EF1a, E. coli lac, E. coli trp, phage lambda PL, phage lambda PR, T3, T7, cytomegalovirus (CMV) immediate early, herpes simplex virus (HSV) thymidine kinase, alpha-lactalbumin, and mouse metallothionein-I promoter sequences. In some preferred embodiments the promoter sequence is oriented at the dock site so that the promoter will drive expression from an inserted nucleic acid construct. In some preferred embodiments, the promoter is oriented 5′ to the docking site. In some particularly preferred embodiments, the promoter is a SIN LTR. In these embodiments, the SIN-LTR and EPR are positioned 5′ to the dock site and a SIN LTR is positioned 3′ to the dock site.

Accordingly, in some preferred embodiments, nucleic acid constructs comprise an insertion element. Preferably the insertion element may be located 5′ to the first promoter, 3′ to the poly A signal sequence, between the first promoter and the poly A signal sequence, between the selectable marker and the second promoter sequence, and both 5′ to the first promoter and 3′ to the poly A signal sequence. Suitable constructs are shown in the following non-limiting examples:
expression construct insertion element—first promoter sequence (optional depending on whether the dock site already comprises an exogenous promoter sequence)—selectable marker sequence—second (i.e., internal) promoter sequence—nucleic acid sequence encoding a first protein of interest—poly A signal sequence
first promoter sequence (optional depending on whether the dock site already comprises an exogenous promoter sequence)—selectable marker sequence—second promoter sequence—nucleic acid sequence encoding a first protein of interest—poly A signal sequence—expression construct insertion element
expression construct insertion element—first promoter sequence (optional depending on whether the dock site already comprises an exogenous promoter sequence)—selectable marker sequence—second promoter sequence—nucleic acid sequence encoding a first protein of interest—poly A signal sequence—expression construct insertion element.
first promoter sequence (optional depending on whether the dock site already comprises an exogenous promoter sequence)—selectable marker sequence—expression construct insertion element—second promoter sequence—nucleic acid sequence encoding a first protein of interest—poly A signal sequence.

In some preferred embodiments, the constructs may include nucleic acid sequences encoding multiple proteins of interest, for example 2, 3, 4 or 5 proteins of interest. Suitable constructs for expressing two proteins of interest are shown in the following nonlimiting examples.
expression construct insertion element—first promoter sequence (optional depending on whether the dock site already comprises an exogenous promoter sequence)—selectable marker sequence—second (i.e., internal) promoter sequence—nucleic acid sequence encoding a first protein of interest—WPRE (optional)—poly A signal sequence—third promoter sequence or IRES—nucleic acid sequence encoding a second protein of interest—WPRE (optional)—poly A signal sequence
first promoter sequence (optional depending on whether the dock site already comprises an exogenous promoter sequence)—selectable marker sequence—second promoter sequence—nucleic acid sequence encoding a first protein of interest—WPRE (optional)—poly A signal sequence—third promoter sequence—intron (optional)—nucleic acid sequence encoding a second protein of interest—WPRE (optional)—poly A signal sequence—expression construct insertion element
expression construct insertion element—first promoter sequence (optional depending on whether the dock site already comprises an exogenous promoter sequence)—selectable marker sequence—second promoter sequence—nucleic acid sequence encoding a first protein of interest—WPRE (optional)—poly A signal sequence—third promoter sequence—intron (optional)—nucleic acid sequence encoding a second protein of interest— WPRE (optional)—poly A signal sequence—expression construct insertion element.
first promoter sequence (optional depending on whether the dock site already comprises an exogenous promoter sequence)—selectable marker sequence—expression construct insertion element—second promoter sequence—nucleic acid sequence encoding a first protein of interest—WPRE—poly A signal sequence—third promoter sequence or IRES—nucleic acid sequence encoding a second protein of interest—WPRE—poly A signal sequence
expression construct insertion element—first promoter sequence (optional depending on whether the dock site already comprises an exogenous promoter sequence)—selectable marker sequence—second promoter sequence—nucleic acid sequence encoding a first protein of interest— WPRE (optional)—poly A signal sequence—third promoter sequence—nucleic acid sequence encoding a second protein of interest—WPRE (optional)—poly A signal sequence—expression construct insertion element.
expression construct insertion element—first promoter sequence (optional depending on whether the dock site already comprises an exogenous promoter sequence)—selectable marker sequence—second promoter sequence—nucleic acid sequence encoding a first protein of interest—WPRE (optional)—poly A signal sequence—third promoter sequence—intron—nucleic acid sequence encoding a second protein of interest— WPRE (optional)—poly A signal sequence—expression construct insertion element.

In some preferred embodiments, the first protein of interest is one of an antibody heavy and light chain and the second protein of interest is the other of an antibody heavy and light chain.

However, any suitable proteins of interest may be expressed via the host cells, constructs and systems of the present invention. Exemplary proteins of interest include immunoglobulins, single chain antibodies, anticoagulant proteins, blood factor proteins, bone morphogenetic proteins, engineered protein scaffolds, enzymes, Fc fusion proteins, growth factors, hormones, interferons, interleukins, antigens, and thrombolytic proteins. In other preferred embodiments, the constructs of the present invention may be utilized to express viral vectors. In these embodiments, the protein of interest sequence described in the exemplary vectors above is replaced with a nucleic acid sequence encoding a viral vector backbone. Viral vectors that may be included in the constructs of the present invention include, but are not limited to, retroviral vectors, lentiviral vectors, adenoviral vectors and AAV vectors. In some preferred embodiments, the retroviral vectors themselves include a nucleic acid sequence encoding a protein of interest as described above that is expressed by the vector. In some particularly preferred embodiments, the protein of interest that is expressed by the vector is an antigen sequence for use in a vaccine.

In some preferred embodiments, the insertion elements are elements that find use in conjunction with or are recognized by transposons, integrases, recombinases or CRISPR systems. Suitable insertion elements include, but are not limited to, inverted terminal repeats, integrase attachment sites (att), and homologous recombination arms which in the context of the constructs described herein can be described as homologous recombination insertion elements.

In some preferred embodiments, the nucleic acid constructs of the present invention comprise transposon insertion elements, preferably inverted terminal repeats that are recognized by transposons. In some preferred embodiments, the inverted terminal repeats are positioned at both the 5′ and 3′ ends of the construct. Transposons are mobile genetic elements that can move or transpose from one location another in the genome. Transposition within the genome is controlled by a transposase enzyme that is encoded by the transposon. Many examples of transposons are known in the art, including, but not limited to, Tn5 (See e.g., de la Cruz et al., J. Bact. 175: 6932-38 [1993], Tn7 (See e.g., Craig, Curr. Topics Microbiol. Immunol. 204: 27-48 [1996]), and Tn10 (See e.g., Morisato and Kleckner, Cell 51:101-111 [1987) transpose systems as well as piggyback transposase systems, sleeping beauty transposase systems, Mos1 transposase systems, To12 transposase systems, and Leapin transposase systems. The ability of transposons to integrate into genomes has been utilized to create transposon vectors (See, e.g., U.S. Pat. Nos. 5,719,055; 5,968,785; 5,958,775; and 6,027,722; all of which are incorporated herein by reference, as well as those supplied by System Biosciences (Palo Alto, Calif.; piggybac system), Creative Biolabs (Shirley, N.Y.; sleeping beauty system), and ATUM (Newark, Calif.; Leapin system)).

Transposition involves an ordered series of events: (1) sequence-specific binding of transposase to the terminal inverted repeats (IRs) present at the ends of the transposon, (2) cleavage of both strands of DNA at each end of the transposon, (3) synapsis of the ends by transposase-transposase interactions, (4) capture of the target DNA and (5) strand transfer to insert the element into the target.

Transposases are members of the retroviral integrase superfamily of proteins. Despite the structural similarities in their catalytic domains, these proteins carry out phosphoryl transfer reactions with different specificities. Some cleave only one strand of DNA, while RNase H cleaves one strand of RNA in an RNA:DNA hybrid duplex. Others generate double-strand DNA breaks, and a variety of mechanisms are employed. The transposases of the bacterial transposons Tn5 and Tn10 carry out first-strand cleavage by hydrolysis to form a 3′ hydroxyl (3′OH) at each end of the element, while the second strand is cleaved by trans-esterification using this 3′OH as the attacking nucleophile. This forms a DNA hairpin at each end of the element, which is hydrolysed by transposase to regenerate the 3′OH required for strand transfer. V(D)J recombination and transposition of the eukaryotic element Hermes, a member of the hAT family, proceed by a similar mechanism, except that the order of strand cleavage is reversed and a hairpin is formed on the flanking, rather than on the excised, DNA. Another bacterial transposon, Tn7, utilizes TnsB to perform first-strand cleavage and recruits a second protein, TnsA, to cleave the nontransferred strand.

Because transposons are not infectious, transposon vectors are introduced into host cells via methods known in the art (e.g., electroporation, lipofection, or microinjection). Therefore, the ratio of transposon vectors to host cells may be adjusted to provide the desired multiplicity of infection to produce the high copy number host cells. Transposon vectors suitable for use in the present invention generally comprise a nucleic acid encoding a protein of interest interposed between two transposon insertion sequences. Some vectors also comprise a nucleic acid sequence encoding a transposase enzyme. In these vectors, one of the insertion sequences is positioned between the transposase enzyme and the nucleic acid encoding the protein of interest so that it is not incorporated into the genome of the host cell during recombination. Alternatively, the transposase enzyme may be provided by a suitable method (e.g., lipofection or microinjection).

In some preferred embodiments, the nucleic acid constructs of the present invention comprise a recombinase insertion element that is recognized by a recombinase. Suitable recombinase insertion elements include, but are not limited to, attachment sites (aat), LoxP sites and MMLV LTR sequences.

In some preferred embodiments, the recombinase insertion element is attB and is used in conjunction with phiC31 integrase (BioCat GmbH, Heidelberg, Del. or System Biosciences, Palo Alto, Calif.)). The phiC31 integrase is a sequence-specific recombinase encoded within the genome of the bacteriophage phiC31. The phiC31 integrase mediates recombination between two 34 base pair sequences termed attachment sites (att), one found in the phage and the other in the host. This serine integrase has been shown to function efficiently in many different cell types including mammalian cells. In the presence of phiC31 integrase, an attB-containing donor plasmid can be unidirectional integrated into a target genome through recombination at sites with sequence similarity to the native attP site (termed pseudo-attP sites). phiC31 integrase can integrate a plasmid of any size, as a single copy, and requires no cofactors. The integrated transgenes are stably expressed and heritable.

In still other preferred embodiments, the insertion element is a nucleic acid sequence homologous to a target site in a chromosome such as a chromosome in a host cell and used in conjunction with a recombinase or systems such as CRISPR. Suitable recombinase-based systems include CRE-Lox, FLP-FRT, and lambda recombinase systems. In general, the nucleic acid sequence that is homologous to a target site in a chromosome will be from 30 to 1000 bases in length.

In some preferred embodiments, the recombinase insertion element is a lox sequence. Cre-Lox recombination is a site-specific recombinase technology, used to carry out deletions, insertions, translocations and inversions at specific sites in the DNA of cells. It allows the DNA modification to be targeted to a specific cell type or be triggered by a specific external stimulus. It is implemented both in eukaryotic and prokaryotic systems. The Cre-lox recombination system has been particularly useful to help neuroscientists to study the brain in which complex cell types and neural circuits come together to generate cognition and behaviors. The system consists of a single enzyme, Cre recombinase, that recombines a pair of short target sequences called the Lox sequences. This system can be implemented without inserting any extra supporting proteins or sequences. The Cre enzyme and the original Lox site called the LoxP sequence are derived from bacteriophage P1. See, e.g., Targeted integration of DNA using mutant lox sites in embryonic stem cells. Araki, et al. Nucleic Acids Res, February 1997, Vol. 25, Issue 4, pp. 868-872; High-Resolution Labeling and Functional Manipulation of Specific Neuron Types in Mouse Brain by Cre-Activated Viral Gene Expression. Kuhlman, et al. PLos One, April 2008, Vol. 3, e2005; When reverse genetics meets physiology: the use of site-specific recombinases in mice. Tronche, et al. FEBS Letters, August 2002, Vol. 529, Issue 1, pp. 116-121.

In some preferred embodiments, the recombinase insertion element is an FRT sequence. The FLP-FRT recombination system is another site-directed recombination technology very conceptually similar to Cre-lox, with flippase (Flp) and the short flippase recognition target (FRT) site being analogous to Cre and loxP, respectively. See, e.g., Candice et al., Cre/loxP, Flp/FRT Systems and Pluripotent Stem Cell Lines (2012) Topics in Current Genetics, vol 23. The FLP-FRT technology can be an effective alternative to Cre-lox, and has also been used in conjunction with it, allowing for two separate recombination events to be controlled in parallel.

In still other preferred embodiments, the nucleic acid constructs of the present invention may used in conjunction with CRISPR homologous recombination (HDR) systems. In these systems, the HDR insertion elements comprise homology arms that are homologous to or base pair with target sequences in the genome. HDR is initiated by the presence of double strand breaks (DSBs) in DNA. The CRISPR/Cas9 system is preferably used to create targeted double stranded breaks via a guide RNA sequence so that the nucleic acid construct of the invention can be inserted. See, e.g., Zhang et al., Efficient precise knockin with a double cut HDR donor after CRISPR/Cas9-mediated double-stranded DNA cleavage (2017) Genome Biol. 18:35; Mali et al., Cas9 as a versatile tool for engineering biology. Nature Methods10, 957-963 (2013); Mali et al., RNA-Guided Human Genome Engineering via Cas9. Science339(6121), 823-826 (2013); Ran et al., Double nicking by RNA-guided CRISPR Cas9 for enhanced genome editing specificity. Cell, 155(2), 479-480(2013). Suitable guide RNA sequences (gRNAs) may be designed as is known in the art. In some preferred embodiments, CRISPR systems for HDR utilize either one or two guide sequences. When one guide RNA sequence is utilized, it preferred to use a nuclease such as a Cas9 nuclease which makes a single double stranded break guided by the guide RNA sequence. When two guide sequences are utilized, it is preferred to use a nickase, which can be a mutated Cas9 nuclease which only makes single stranded breaks in the target DNA sequence guided by each of the guide RNA sequences. The single stranded breaks are preferably positioned at staggered points on different strands (i.e., the sense and antisense strands) of the target DNA sequence. This arrangement generally improves HDR efficiency.

In general, “CRISPR system” refers collectively to transcripts and other elements involved in the expression of or directing the activity of CRISPR-associated (“Cas”) genes, including sequences encoding a Cas gene, a tracr (trans-activating CRISPR) sequence (e.g. tracrRNA or an active partial tracrRNA), a tracr-mate sequence (encompassing a “direct repeat” and a tracrRNA-processed partial direct repeat in the context of an endogenous CRISPR system), a guide sequence (also referred to as a “spacer” in the context of an endogenous CRISPR system), or other sequences and transcripts from a CRISPR locus. In some embodiments, one or more elements of a CRISPR system is derived from a type I, type II, or type III CRISPR system. In some embodiments, one or more elements of a CRISPR system is derived from a particular organism comprising an endogenous CRISPR system, such as Streptococcus pyogenes. In general, a CRISPR system is characterized by elements that promote the formation of a CRISPR complex at the site of a target sequence (also referred to as a protospacer in the context of an endogenous CRISPR system). In the context of formation of a CRISPR complex, “target sequence” refers to a sequence to which a guide sequence is designed to have complementarity, where hybridization between a target sequence and a guide sequence promotes the formation of a CRISPR complex. Full complementarity is not necessarily required, provided there is sufficient complementarity to cause hybridization and promote formation of a CRISPR complex. A target sequence may comprise any polynucleotide, such as DNA or RNA polynucleotides. In some embodiments, a target sequence is located in the nucleus or cytoplasm of a cell. In some embodiments, the target sequence may be within an organelle of a eukaryotic cell, for example, mitochondrion or chloroplast. A sequence or template that may be used for recombination into the targeted locus comprising the target sequences is referred to as an “editing template” or “editing polynucleotide” or “editing sequence”. In aspects of the invention, an exogenous template polynucleotide may be referred to as an editing template. In an aspect of the invention the recombination is homologous recombination.

Typically, in the context of an endogenous CRISPR system, formation of a CRISPR complex (comprising a guide sequence hybridized to a target sequence and complexed with one or more Cas proteins) results in cleavage of one or both strands in or near (e.g. within 1, 2, 3, 4, 5, 6, 7, 8, 9, 10, 20, 50, or more base pairs from) the target sequence. Without wishing to be bound by theory, the tracr sequence, which may comprise or consist of all or a portion of a wild-type tracr sequence (e.g. about or more than about 20, 26, 32, 45, 48, 54, 63, 67, 85, or more nucleotides of a wild-type tracr sequence), may also form part of a CRISPR complex, such as by hybridization along at least a portion of the tracr sequence to all or a portion of a tracr mate sequence that is operably linked to the guide sequence. In some embodiments, the tracr sequence has sufficient complementarity to a tracr mate sequence to hybridize and participate in formation of a CRISPR complex. As with the target sequence, it is believed that complete complementarity is not needed, provided there is sufficient to be functional. In some embodiments, the tracr sequence has at least 50%, 60%, 70%, 80%, 90%, 95% or 99% of sequence complementarity along the length of the tracr mate sequence when optimally aligned. In some embodiments, one or more vectors driving expression of one or more elements of a CRISPR system are introduced into a host cell such that expression of the elements of the CRISPR system direct formation of a CRISPR complex at one or more target sites. For example, a Cas enzyme, a guide sequence linked to a tracr-mate sequence, and a tracr sequence could each be operably linked to separate regulatory elements on separate vectors. Alternatively, two or more of the elements expressed from the same or different regulatory elements, may be combined in a single vector, with one or more additional vectors providing any components of the CRISPR system not included in the first vector. CRISPR system elements that are combined in a single vector may be arranged in any suitable orientation, such as one element located 5′ with respect to (“upstream” of) or 3′ with respect to (“downstream” of) a second element. The coding sequence of one element may be located on the same or opposite strand of the coding sequence of a second element, and oriented in the same or opposite direction. In some embodiments, a single promoter drives expression of a transcript encoding a CRISPR enzyme and one or more of the guide sequence, tracr mate sequence (optionally operably linked to the guide sequence), and a tracr sequence embedded within one or more intron sequences (e.g. each in a different intron, two or more in at least one intron, or all in a single intron). In some embodiments, the CRISPR enzyme, guide sequence, tracr mate sequence, and tracr sequence are operably linked to and expressed from the same promoter.

Non-limiting examples of Cas proteins useful in the present invention include Cas1, Cas1B, Cas2, Cas3, Cas4, Cas5, Cas6, Cas7, Cas8, Cas9 (also known as Csn1 and Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, homologs thereof, or modified versions thereof. These enzymes are known; for example, the amino acid sequence of S. pyogenes Cas9 protein may be found in the SwissProt database under accession number Q99ZW2. In some embodiments, the unmodified CRISPR enzyme has DNA cleavage activity, such as Cas9. In some embodiments the CRISPR enzyme is Cas9, and may be Cas9 from S. pyogenes or S. pneumoniae. In some embodiments, the CRISPR enzyme directs cleavage of one or both strands at the location of a target sequence, such as within the target sequence and/or within the complement of the target sequence. In some embodiments, the CRISPR enzyme directs cleavage of one or both strands within about 1, 2, 3, 4, 5, 6, 7, 8, 9, 10, 15, 20, 25, 50, 100, 200, 500, or more base pairs from the first or last nucleotide of a target sequence. In some embodiments, a vector encodes a CRISPR enzyme that is mutated to with respect to a corresponding wild-type enzyme such that the mutated CRISPR enzyme lacks the ability to cleave one or both strands of a target polynucleotide containing a target sequence. For example, an aspartate-to-alanine substitution (D10A) in the RuvC I catalytic domain of Cas9 from S. pyogenes converts Cas9 from a nuclease that cleaves both strands to a nickase (cleaves a single strand). Other examples of mutations that render Cas9 a nickase include, without limitation, H840A, N854A, and N863A. In aspects of the invention, nickases may be used for genome editing via homologous recombination.

In some preferred embodiments, the HDR insertion element comprises an AAVS1 safe harbor locus and is used in conjunction with Rep 78 integrase. In particularly preferred embodiments, the HDR insertion element comprises homology arms that base pair with the AAVS1 safe harbor locus. The adeno-associated virus serotype 2 (AAV2) Rep 78 protein is a strand-specific endonuclease (nickase) that promotes site-specific integration of transgene sequences bearing homology arms corresponding to the AAVS1 safe harbor locus. See, e.g., Ramachandra et al., Efficient recombinase-mediated cassette exchange at the AAVS1 locus in human embryonic stem cells using baculoviral vectors (2011) Nucleic Acids Research, 39(16):e107; WO1998027207).

As indicated above, in some preferred embodiments, the nucleic acid constructs of the present invention comprise first and second promoter sequences. The first and second promoter sequences may be the same or different. Suitable first and second promoter sequences include, but are not limited to the MMLV LTR promoter, the MoMuSV LTR promoter, the RSV LTR promoter, the SIN LTR promoter, the SV40 promoter, cytomegalovirus (CMV) immediate early promoter, herpes simplex virus (HSV) thymidine kinase promoter, alpha-lactalbumin promoter, mouse metallothionein-I promoter, dihydrofolate reductase promoter, the (3-actin promoter, phosphoglycerol kinase (PGK) promoter, and the EF1α promoter sequences, and combinations thereof. In some preferred embodiments, the first promoter sequence is not a retroviral LTR promoter, i.e., the first promoter is promoter sequence other than a retroviral LTR promoter sequence. However, when the promoter is a retroviral promoter sequence, it may be a SIN (self-inactivating) LTR promoter sequence. See, e.g., co-pending application PCT/US2019/064423, which is incorporated herein by reference in its entirety. Suitable Sin LTR promotors are known in the art and are prepared by removing either all or a portion of the U3 region of the LTR.

As described in PCT/US2019/064423, in some preferred embodiments the first promoter which drives selectable marker is a weak promoter. In some preferred embodiments, a weak promoter is a promoter, preferably a constitutive promoter, that has activity that equal to or less than the activity of the SIN LTR promoter in a host of interest (e.g., a CHO cell) when operably linked to a selectable maker sequence. In still other preferred embodiments, a weak promoter is a promoter, preferably a constitutive promoter, that has activity that equal to or less than the activity of the human Ubiquitin C (UBC) promoter in a host of interest (e.g., a CHO cell) when operably linked to a selectable maker sequence. Suitable methods for assessing promoter strength are known in the art. See, e.g., Dandindorj et al. (2014) A Comparative Analysis of Constitutive Promoters Located in Adeno-Associated Viral Vectors, PLoS One 9(8): e106472; Zhang and Baum (2005) Evaluation of Viral and Mammalian Promoters for Use in Gene Delivery to Salivary Glands Mol. Ther. 12(3):528-536; Qin et al. (2010) Systematic Comparison of Constitutive Promoters and the Doxycycline-Inducible Promoter PLoS 5(5): e10611; Jeyaseelan et al. (2001) Real-time detection of gene promoter activity: quantitation of toxin gene transcription, Nucleic Acids Research. 29 (12): 58e-58. In some embodiments, weak promoters have been altered to reduce promoter activity. Accordingly, in some preferred embodiments, the present invention provides vector(s) for expression of a protein of interest comprising a nucleic acid sequence encoding a selectable marker in operable association with a first weak promoter sequence or promoter sequence that has been altered to reduce promoter activity as compared to a non-altered or wild-type version of the first promoter sequence and a nucleic acid sequence encoding the protein of interest operably linked to a second promoter sequence. The SIN LTR promoter sequence is one such example. Other promoter sequences described above may also be altered to reduce activity and provide a weak promoter or the weak promoter may be naturally occurring weak promoter such as the UBC promoter.

In some preferred embodiments, the nucleic acid constructs include a selectable marker. Suitable selectable markers include but are not limited to glutamine synthetase (GS), dihydrofolate reductase (DHFR) and the like. These genes are described in U.S. Pat. Nos. 5,770,359; 5,827,739; 4,399,216; 4,634,665; 5,149,636; and 6,455,275; all of which are incorporated herein by reference. In some preferred embodiments, the selectable marker that is utilized is compatible with a host cell line that is deficient in the production of the enzyme encoded by the selectable marker nucleic acid sequence. Suitable host cell lines are described in more detail below. In other embodiments, the selectable marker is an antibiotic resistance marker, i.e., a gene that produces a protein that provides cells expressing this protein with resistance to an antibiotic. Suitable antibiotic resistance markers include genes that provide resistance to neomycin (neomycin resistance gene (neo)), hygromycin (hygromycin B phosphotransferase gene), puromycin (puromycin N-acetyl-transferase), and the like.

In other embodiments of the present invention, where secretion of the protein of interest is desired, the nucleic acid constructs include a signal peptide sequence in operable association with the protein of interest. The sequences of several suitable signal peptides are known to those in the art, including, but not limited to, those derived from tissue plasminogen activator, human growth hormone, lactoferrin, alpha-casein, and alpha-lactalbumin.

In other embodiments of the present invention, the nucleic acid constructs include an RNA export element (See, e.g., U.S. Pat. Nos. 5,914,267; 6,136,597; and 5,686,120; and WO99/14310, all of which are incorporated herein by reference) either 3′ or 5′ to the nucleic acid sequence encoding the protein of interest. It is contemplated that the use of RNA export elements allows high levels of expression of the protein of interest without incorporating splice signals or introns in the nucleic acid sequence encoding the protein of interest.

In still other embodiments, the nucleic acid constructs include at least one internal ribosome entry site (IRES) sequence. The sequences of several suitable IRES's are available, including, but not limited to, those derived from foot and mouth disease virus (FDV), encephalomyocarditis virus, and poliovirus. The IRES sequence can be interposed between two transcriptional units (e.g., nucleic acids encoding different proteins of interest or subunits of a multi-subunit protein such as an antibody) to form a polycistronic sequence so that the two transcriptional units are transcribed from the same promoter.

The present invention is not limited to expression of any particular protein of interest. In some preferred embodiments, the protein of interest is selected from the group consisting of an Fc-fusion protein, an enzyme, an albumin fusion, a growth factor, a protein receptor, a single chain antibody (scFv), a single chain-Fc (scFv-Fc), a diabody, and minibody (scFv-CH3), Fab, single chain Fab (scFab), an immunoglobulin heavy chain, and an immunoglobulin light chain and other antigen binding proteins.

In some preferred embodiments, the nucleic acid constructs are incorporated into a nucleic acid expression vector. Vectors include, but are not limited to, nucleic acid molecules that are single-stranded, double-stranded, or partially double-stranded; nucleic acid molecules that comprise one or more free ends, no free ends (e.g. circular); nucleic acid molecules that comprise DNA, RNA, or both; and other varieties of polynucleotides known in the art. One type of vector is a “plasmid,” which refers to a circular double stranded DNA loop into which additional DNA segments can be inserted, such as by standard molecular cloning techniques. Another type of vector is a viral vector, wherein virally-derived DNA or RNA sequences are present in the vector for packaging into a virus (e.g. retroviruses, replication defective retroviruses, adenoviruses, replication defective adenoviruses, and adeno-associated viruses). Viral vectors also include polynucleotides carried by a virus for transfection into a host cell. Certain vectors are capable of autonomous replication in a host cell into which they are introduced (e.g. bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively-linked. Such vectors are referred to herein as “expression vectors.” Common expression vectors of utility in recombinant DNA techniques are often in the form of plasmids.

Accordingly, suitable nucleic acid expression vectors include, but are not limited to, transposon vectors as described above, as well as plasmid vectors, retroviral vectors, lentiviral vectors, AAV vectors, phage vectors, etc). It is contemplated that any vector may be used as long as it is replicable and viable in the host. In preferred embodiments, the vectors are mammalian expression vectors that comprise among other elements described herein an origin of replication, a suitable promoter and enhancer, and also any necessary ribosome binding sites, polyadenylation sites, splice donor and acceptor sites, transcriptional termination sequences, and 5′ flanking non-transcribed sequences.

Suitable plasmid vectors that may be adapted to incorporate the nucleic acid constructs of the present invention include specific plasmids systems for transposon vectors, FLP-FLT systems, Cre-lox systems, CRISPR-Cas9 systems, remonbinase systems and integrase systems as well as plasmid vectors derived from pCIneo, pVAX1, pACT, Gateway plamids, pAdvantage, pBIND, pG5luc, pTNT, pTarget, pCat3, pSI, pCMV, pSV and the like.

In some embodiments, the vectors are retroviral vectors. The most commonly used recombinant retroviral vectors are derived from the amphotropic Moloney murine leukemia virus (MoMLV) (See e.g., Miller and Baltimore Mol. Cell. Biol. 6:2895 [1986]). The MoMLV system has several advantages: 1) this specific retrovirus can infect many different cell types, 2) established packaging cell lines are available for the production of recombinant MoMLV viral particles and 3) the transferred genes are permanently integrated into the target cell chromosome. The established MoMLV vector systems comprise a DNA vector containing a small portion of the retroviral sequence (e.g., the viral long terminal repeat or “LTR” and the packaging or “psi” signal) and a packaging cell line. The gene to be transferred is inserted into the DNA vector. The viral sequences present on the DNA vector provide the signals necessary for the insertion or packaging of the vector RNA into the viral particle and for the expression of the inserted gene. The packaging cell line provides the proteins required for particle assembly (Markowitz et al., J. Virol. 62:1120 [1988]).

In some preferred embodiments, the retroviral vectors are pseudotyped, and for example utilize the G protein of VSV as the membrane associated protein. Unlike retroviral envelope proteins that bind to a specific cell surface protein receptor to gain entry into a cell, the VSV G protein interacts with a phospholipid component of the plasma membrane (Mastromarino et al., J. Gen. Virol. 68:2359 [1977]). Because entry of VSV into a cell is not dependent upon the presence of specific protein receptors, VSV has an extremely broad host range. Pseudotyped retroviral vectors bearing the VSV G protein have an altered host range characteristic of VSV (i.e., they can infect almost all species of vertebrate, invertebrate and insect cells). Importantly, VSV G-pseudotyped retroviral vectors can be concentrated 2000-fold or more by ultracentrifugation without significant loss of infectivity (Burns et al. Proc. Natl. Acad. Sci. USA 90:8033 [1993]).

In some preferred embodiments, the vectors are lentiviral vectors. The lentiviruses (e.g., equine infectious anemia virus, caprine arthritis-encephalitis virus, human immunodeficiency virus) are a subfamily of retroviruses that are able to integrate into non-dividing cells. The lentiviral genome and the proviral DNA have the three genes found in all retroviruses: gag, pol, and env, which are flanked by two LTR sequences. The gag gene encodes the internal structural proteins (e.g., matrix, capsid, and nucleocapsid proteins); the pol gene encodes the reverse transcriptase, protease, and integrase proteins; and the pol gene encodes the viral envelope glycoproteins. The 5′ and 3′ LTRs control transcription and polyadenylation of the viral RNAs. Additional genes in the lentiviral genome include the vif, vpr, tat, rev, vpu, nef, and vpx genes.

A variety of lentiviral vectors and packaging cell lines are known in the art and find use in the present invention (See, e.g., U.S. Pat. Nos. 5,994,136 and 6,013,516, both of which are herein incorporated by reference). Furthermore, the VSV G protein has also been used to pseudotype retroviral vectors based upon the human immunodeficiency virus (HIV) (Naldini et al., Science 272:263 [1996]). Thus, the VSV G protein may be used to generate a variety of pseudotyped retroviral vectors and is not limited to vectors based on MoMLV. The lentiviral vectors may also be modified as described above to contain various regulatory sequences (e.g., signal peptide sequences, RNA export elements, and IRES's). After the lentiviral vectors are produced, they may be used to transfect host cells as described above for retroviral vectors.

In some preferred embodiments, the vectors are adeno-associated virus (AAV) vectors. The AAV genome is composed of a linear, single-stranded DNA molecule that contains approximately 4680 bases. The genome includes inverted terminal repeats (ITRs) at each end that function in cis as origins of DNA replication and as packaging signals for the virus. The internal nonrepeated portion of the genome includes two large open reading frames, known as the AAV rep and cap regions, respectively. These regions code for the viral proteins involved in replication and packaging of the virion. A family of at least four viral proteins are synthesized from the AAV rep region, Rep 78, Rep 68, Rep 52 and Rep 40, named according to their apparent molecular weight. The AAV cap region encodes at least three proteins, VP1, VP2 and VP3 (for a detailed description of the AAV genome, see e.g., Muzyczka, Current Topics Microbiol. Immunol. 158:97-129 [1992]; Kotin, Human Gene Therapy 5:793-801 [1994]).

AAV requires coinfection with an unrelated helper virus, such as adenovirus, a herpesvirus or vaccinia, in order for a productive infection to occur. In the absence of such coinfection, AAV establishes a latent state by insertion of its genome into a host cell chromosome. Subsequent infection by a helper virus rescues the integrated copy, which can then replicate to produce infectious viral progeny. Unlike the non-pseudotyped retroviruses, AAV has a wide host range and is able to replicate in cells from any species so long as there is coinfection with a helper virus that will also multiply in that species. Thus, for example, human AAV will replicate in canine cells coinfected with a canine adenovirus. Furthermore, unlike the retroviruses, AAV is not associated with any human or animal disease, does not appear to alter the biological properties of the host cell upon integration and is able to integrate into nondividing cells. It has also recently been found that AAV is capable of site-specific integration into a host cell genome.

In light of the above-described properties, a number of recombinant AAV vectors have been developed for gene delivery (See, e.g., U.S. Pat. Nos. 5,173,414; 5,139,941; WO 92/01070 and WO 93/03769, both of which are incorporated herein by reference; Lebkowski et al., Molec. Cell. Biol. 8:3988-3996 [1988]; Carter, Current Opinion in Biotechnology 3:533-539 [1992]; Muzyczka, Current Topics in Microbiol. and Immunol. 158:97-129 [1992]; Kotin, (1994) Human Gene Therapy 5:793-801; Shelling and Smith, Gene Therapy 1:165-169 [1994]; and Zhou et al., J. Exp. Med. 179:1867-1875 [1994]).

Recombinant AAV virions can be produced in a suitable host cell that has been transfected with both an AAV helper plasmid and an AAV vector. An AAV helper plasmid generally includes AAV rep and cap coding regions, but lacks AAV ITRs. Accordingly, the helper plasmid can neither replicate nor package itself An AAV vector generally includes a selected gene of interest bounded by AAV ITRs that provide for viral replication and packaging functions. Both the helper plasmid and the AAV vector bearing the selected gene are introduced into a suitable host cell by transient transfection. The transfected cell is then infected with a helper virus, such as an adenovirus, which transactivates the AAV promoters present on the helper plasmid that direct the transcription and translation of AAV rep and cap regions. Recombinant AAV virions harboring the selected gene are formed and can be purified from the preparation. Once the AAV vectors are produced, they may be used to transfect (See, e.g., U.S. Pat. No. 5,843,742, herein incorporated by reference) host cells at the desired multiplicity of infection to produce high copy number host cells. As will be understood by those skilled in the art, the AAV vectors may also be modified as described above to contain various regulatory sequences (e.g., signal peptide sequences, RNA export elements, and IRES's).

In some embodiments, the present invention provides host cells and host cell culture wherein the host cells express the protein of interest from the nucleic acid constructs described above. In preferred embodiment, the host cells a mammalian host cells. A number of mammalian host cell lines are known in the art. In general, these host cells are capable of growth and survival when placed in either monolayer culture or in suspension culture in a medium containing the appropriate nutrients and growth factors, as is described in more detail below. Typically, the cells are capable of expressing and secreting large quantities of a particular protein of interest into the culture medium. Examples of suitable mammalian host cells include, but are not limited to Chinese hamster ovary cells (CHO-K1, ATCC CC1-61); bovine mammary epithelial cells (ATCC CRL 10274; bovine mammary epithelial cells); monkey kidney CV1 line transformed by SV40 (COS-7, ATCC CRL 1651); human embryonic kidney line (293 or 293 cells subcloned for growth in suspension culture; see, e.g., Graham et al., J. Gen Virol., 36:59 [1977]); baby hamster kidney cells (BHK, ATCC CCL 10); mouse sertoli cells (TM4, Mather, Biol. Reprod. 23:243-251 [1980]); monkey kidney cells (CV1 ATCC CCL 70); African green monkey kidney cells (VERO-76, ATCC CRL-1587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCL51); TRI cells (Mather et al., Annals N.Y. Acad. Sci., 383:44-68) [1982]); MRC 5 cells; FS4 cells; rat fibroblasts (208F cells); MDBK cells (bovine kidney cells); CAP (CEVEC's Amniocyte Production) cells; and a human hepatoma line (Hep G2).

In some particularly preferred embodiments, the host cells are modified so that they are deficient, or are naturally deficient, in an enzyme activity that is required for growth or survival of the cells in the presence of a selection agent and which is provided by the selectable marker. For example, Chinese Hamster Ovary (CHO) cells have been modified to be deficient for GS. In some preferred embodiments where vector includes a GS selectable marker, the host cell line is deficient in GS. In some particularly preferred embodiments, the GS deficient host cell line is the CHOZN® GS^{-/-} cell line available from Merck KGaA. In other embodiments, where the selectable marker is, for example, DHFR, the cell line may preferably be deficient for DHFR activity (i.e., DHFR⁻). Suitable DHFR-cell lines include but are not limited to CHO-DG44 and derivatives thereof.

The nucleic acid constructs and vectors of the present invention may be introduced into host cells by any suitable means such as by transfection, transformation or transduction. In some embodiments, after transfection or transduction, the cells are allowed to multiply, and are then trypsinized and replated. Individual colonies are then selected to provide clonally selected cell lines. In still further embodiments, the clonally selected cell lines are screened by Southern blotting or PCR assays to verify that the desired number of integration events has occurred. It is also contemplated that clonal selection allows the identification of superior protein producing cell lines. In other embodiments, the cells are not clonally selected following transfection.

In some embodiments, the host cells are transfected with vectors encoding different proteins of interest. The vectors encoding different proteins of interest can be used to transfect the cells at the same time (e.g., the host cells are exposed to a solution containing vectors encoding different proteins of interest) or the transfection can be serial (e.g., the host cells are first transfected with a vector encoding a first protein of interest, a period of time is allowed to pass, and the host cells are then transfected with a vector encoding a second protein of interest). In some preferred embodiments, the host cells are transfected with an integrating vector encoding a first protein of interest, high expressing cell lines containing multiple integrated copies of the integrating vector are selected (e.g., clonally selected), and the selected cell line is transfected with an integrating vector encoding a second protein of interest. This process may be repeated to introduce multiple proteins of interest. In some embodiments, the multiplicities of infection may be manipulated (e.g., increased or decreased) to increase or decrease the expression of the protein of interest. Likewise, the different promoters may be utilized to vary the expression of the proteins of interest. It is contemplated that these transfection methods can be used to construct host cell lines containing an entire exogenous metabolic pathway or to provide host cells with an increased capability to process proteins (e.g., the host cells can be provided with enzymes necessary for post-translational modification).

In some embodiments of the present invention, following transformation of a suitable host strain and growth of the host strain to an appropriate cell density in media, the protein of interest is secreted during culture of the host cells. In some preferred embodiments where amplifiable markers are utilized, it is contemplated that culture of transduced host cells in a medium comprising an inhibitor of the gene. Suitable inhibitors include, but are not limited to methotrexate for inhibition of DHFR and methionine sulphoximine (Msx) or phosphinothricin for inhibition of GS. It is contemplated that as concentrations of these inhibitors are increased in a cell culture system, cells with higher copy numbers of the amplifiable marker (and thus the genes or genes of interest) or which contain higher-producing insertions are selected.

Accordingly, the host cells containing vectors as described above are preferably cultured according to methods known in the art. Suitable culture conditions for mammalian cells are well known in the art (See e.g., J. Immunol. Methods (1983) 56:221-234 [1983], Animal Cell Culture: A Practical Approach 2nd Ed., Rickwood, D. and Hames, B. D., eds. Oxford University Press, New York [1992]).

The host cell cultures of the present invention are prepared in a media suitable for the particular cell being cultured. Commercially available media such as ActiPro media (HyClone), ExCell Advanced Fed Batch Medium (SAFC), Ham's F10 (Sigma, St. Louis, Mo.), Minimal Essential Medium (MEM, Sigma), RPMI-1640 (Sigma), and Dulbecco's Modified Eagle's Medium (DMEM, Sigma) are exemplary nutrient solutions. Suitable media are also described in U.S. Pat. Nos. 4,767,704; 4,657,866; 4,927,762; 5,122,469; 4,560,655; and WO 90/03430 and WO 87/00195; the disclosures of which are herein incorporated by reference. Any of these media may be supplemented as necessary with serum, hormones and/or other growth factors (such as insulin, transferrin, or epidermal growth factor), salts (such as sodium chloride, calcium, magnesium, and phosphate), buffers (such as HEPES), nucleosides (such as adenosine and thymidine), antibiotics (such as gentamycin (gentamicin), trace elements (defined as inorganic compounds usually present at final concentrations in the micromolar range) lipids (such as linoleic or other fatty acids) and their suitable carriers, and glucose or an equivalent energy source. In some preferred embodiments where selectable markers such as GS are utilized, for example, the media will lack glutamine. Any other necessary supplements may also be included at appropriate concentrations that would be known to those skilled in the art.

The present invention also contemplates the use of a variety of culture systems (e.g., petri dishes, 96 well plates, roller bottles, and bioreactors) for the transfected host cells. For example, the transfected host cells can be cultured in a perfusion system. Perfusion culture refers to providing a continuous flow of culture medium through a culture maintained at high cell density. The cells are suspended and do not require a solid support to grow on. Generally, fresh nutrients must be supplied continuously with concomitant removal of toxic metabolites and, ideally, selective removal of dead cells. Filtering, entrapment and micro-capsulation methods are all suitable for refreshing the culture environment at sufficient rates.

As another example, in some embodiments a fed batch culture procedure can be employed. In the preferred fed batch culture the mammalian host, cells and culture medium are supplied to a culturing vessel initially and additional culture nutrients are fed, continuously or in discrete increments, to the culture during culturing, with or without periodic cell and/or product harvest before termination of culture. The fed batch culture can include, for example, a semi-continuous fed batch culture, wherein periodically whole culture (including cells and medium) is removed and replaced by fresh medium. Fed batch culture is distinguished from simple batch culture in which all components for cell culturing (including the cells and all culture nutrients) are supplied to the culturing vessel at the start of the culturing process. Fed batch culture can be further distinguished from perfusion culturing insofar as the supernatant is not removed from the culturing vessel during the process (in perfusion culturing, the cells are restrained in the culture by, e.g., filtration, encapsulation, anchoring to microcarriers etc. and the culture medium is continuously or intermittently introduced and removed from the culturing vessel). In some particularly preferred embodiments, the batch cultures are performed in roller bottles.

Further, the cells of the culture may be propagated according to any scheme or routine that may be suitable for the particular host cell and the particular production plan contemplated. Therefore, the present invention contemplates a single step or multiple step culture procedure. In a single step culture, the host cells are inoculated into a culture environment and the processes of the instant invention are employed during a single production phase of the cell culture. Alternatively, a multi-stage culture is envisioned. In the multi-stage culture cells may be cultivated in a number of steps or phases. For instance, cells may be grown in a first step or growth phase culture wherein cells, possibly removed from storage, are inoculated into a medium suitable for promoting growth and high viability. The cells may be maintained in the growth phase for a suitable period of time by the addition of fresh medium to the host cell culture.

Fed batch or continuous cell culture conditions are devised to enhance growth of the mammalian cells in the growth phase of the cell culture. In the growth phase cells are grown under conditions and for a period of time that is maximized for growth. Culture conditions, such as temperature, pH, dissolved oxygen (dO2) and the like, are those used with the particular host and will be apparent to the ordinarily skilled artisan. Generally, the pH is adjusted to a level between about 6.5 and 7.5 using either an acid (e.g., CO₂) or a base (e.g., Na2CO3 or NaOH). A suitable temperature range for culturing mammalian cells such as CHO cells is between about 30° to 38° C. and a suitable dO₂ is between 5-90% of air saturation.

Following the polypeptide production phase, the polypeptide of interest is recovered from the culture medium using techniques that are well established in the art. The protein of interest preferably is recovered from the culture medium as a secreted polypeptide (e.g., the secretion of the protein of interest is directed by a signal peptide sequence), although it also may be recovered from host cell lysates. As a first step, the culture medium or lysate is centrifuged to remove particulate cell debris. The polypeptide thereafter is purified from contaminant soluble proteins and polypeptides, with the following procedures being exemplary of suitable purification procedures: by fractionation on immunoaffinity or ion-exchange columns; ethanol precipitation; reverse phase HPLC; chromatography on silica or on a cation-exchange resin such as DEAE; chromatofocusing; SDS-PAGE; ammonium sulfate precipitation; gel filtration using, for example, Sephadex G-75; and protein A Sepharose columns to remove contaminants such as IgG. A protease inhibitor such as phenyl methyl sulfonyl fluoride (PMSF) also may be useful to inhibit proteolytic degradation during purification. Additionally, the protein of interest can be fused in frame to a marker sequence that allows for purification of the protein of interest. Non-limiting examples of marker sequences include a hexa-histidine tag, which may be supplied by a vector, preferably a pQE-9 vector, and a hemagglutinin (HA) tag. The HA tag corresponds to an epitope derived from the influenza hemagglutinin protein (See e.g., Wilson et al., Cell, 37:767 [1984]). One skilled in the art will appreciate that purification methods suitable for the polypeptide of interest may require modification to account for changes in the character of the polypeptide upon expression in recombinant cell culture.

In some preferred embodiments, the nucleic acid constructs are incorporated into systems. In some embodiments, the systems comprise multiple nucleic acid constructs or vectors as described above which are intended for introduction into a host cell. In other preferred embodiments, the systems comprise one or more multiple nucleic acid constructs or vectors as described above which are intended for introduction into a host cell in addition to a nucleic acid or vector that encodes an enzyme that is necessary for incorporation of the nucleic acid constructs into a host cell genome. Exemplary enzymes include, but are not limited to, transposes for use with transposon vector systems, integrases for use in systems which utilize integration sequences such as the PhiC31 system, MMLV systems, and the like, recombinases for use in vector systems such as Cre-loc, FLP-FRT and the like, and Cas9 nucleases for use in CRISP based systems.

### EXPERIMENTAL

The invention provides a unique way of combining the SIN-LTR retroviral expression cassette with the Glutamine Synthase (GS) knock-out CHO cell line system to improve cell line development methods utilizing random integration resulting in higher gene copy number and higher productivity per copy. It further provides an improved and unexpected method for more stringent selection of pools to further improve titer and enrich pools for higher producing clones. It also provides a fast and efficient method for the development of high-producing cell lines through targeted integration of expression cassettes (transgenes) into predefined sites (docks) throughout the CHO genome.

### Example 1

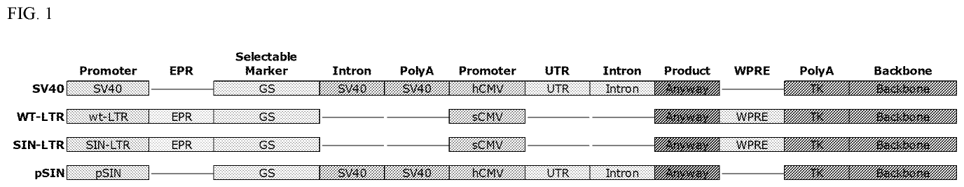

Three pooled cell lines were produced from transient transfection of five independent plasmids (FIG. 1) all designed to express a test protein “Anyway”. These plasmids are referred to by the promoter they utilize to drive GS expression. The first plasmid, SV40, represents the traditional method of cell line development—a plasmid containing a selectable marker gene (GS) driven by the strong SV40 promoter and also containing the SV40 intron and Poly A signal. The second plasmid, WT-LTR, utilizes the proviral wild-type LTR to drive expression of GS expression set up in a context similar to what a GPEx vector insert would look like. Though this is thought to be a relatively strong promoter, the transcript from this promoter does not terminate after GS but rather continues through sCMV, Anyway, and WPRE, utilizing the TK poly A sequence. The third plasmid, SIN-LTR is identical to the second construct except that it contains a truncated version of the LTR, SIN-LTR (Self Inactivating-LTR), that has lower promoter activity. The fourth plasmid, pSIN, is identical to the first plasmid except that instead of a strong promoter driving GS expression, it utilizes the weaker promoter element from SIN-LTR. The fifth plasmid expressed GFP but does not contain the GS gene and therefore serves as a negative control.

Pools generated by transfection of these plasmids were selected for survival in the absence of Glutamine. The selected pools were subjected to generic fed batch production to measure their ability to produce the Anyway protein.

### CHOZN Cell Line Development

Transfection of CHOZn cells: Pooled cell lines containing random integrations of each plasmid were made by transfecting the cells with the indicated plasmid using Expifectamine CHO. 20 ug of plasmid was added to 1 ml of OptiPro medium. 80 ul of Expifectamine CHO was added to 920 ul of OptiPro. These two solutions were mixed for 1 minute, then added to 3 mls of CHO-Gro media containing 30 million CHOZn cells. The cells were incubated overnight at 37 degrees, shaking at 250 RPM. 15 mls of Excell CD Fusion media supplemented with 6 mM Glutamine was added the next morning. Cells were passaged in this media until they recovered from transfection.

Selection of CHOZn cells: Once cells reached >96% viability, they were passaged into Ex-Cell CD Fusion media supplemented with 2% ClonaCell-CHO ACF but without glutamine via a full media replacement. Cells were regularly monitored for viability and viable cell density. Media was replaced weekly until cultured reached 1 million cells per ml and were passaged routinely.

Fed Batch Production: Prior to the fed batch production, each pool was adapted to ActiPro media for at least three passages. For the fed batch production, 50 ml spin tubes were seeded at 600,000 cells per ml in ActiPro media (HyClone) and incubated in a humidified (70-80%) shaking incubator at 250 rpm with 5% CO₂ and temperature of 37° C. (34° C. starting day 5). Cultures were fed six times during the production run using two different feed supplements. Glucose was monitored daily and supplemented if the level dropped below 5 g/L. Cultures were terminated when viabilities were ≤70%.

### Results

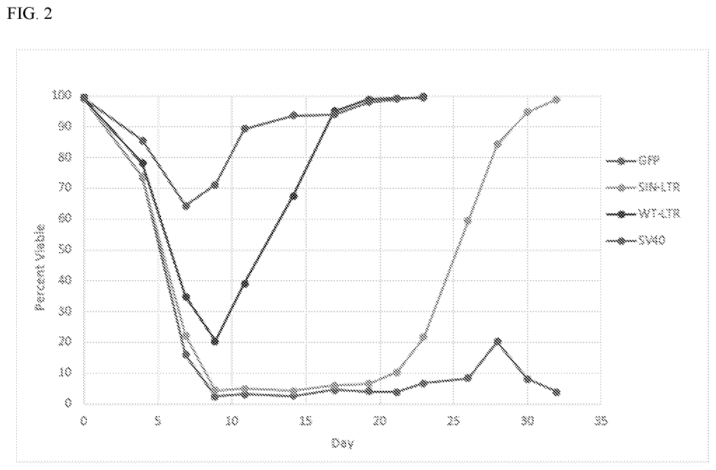

As displayed in FIG. 2, the SV40, WT-LTR, and SIN-LTR pools showed dramatically different selection recovery profiles. SV40 pools showed the fastest recovery (>90% viability), indicating that a relatively large portion of the cells in the unselected pool were resistant to selection. WT-LTR pools slower recovery, indicating a smaller portion of the unselected pool was resistant. SIN-LTR pools showed a markedly delayed recovery indicating a very small portion of the unselected pool was resistant.

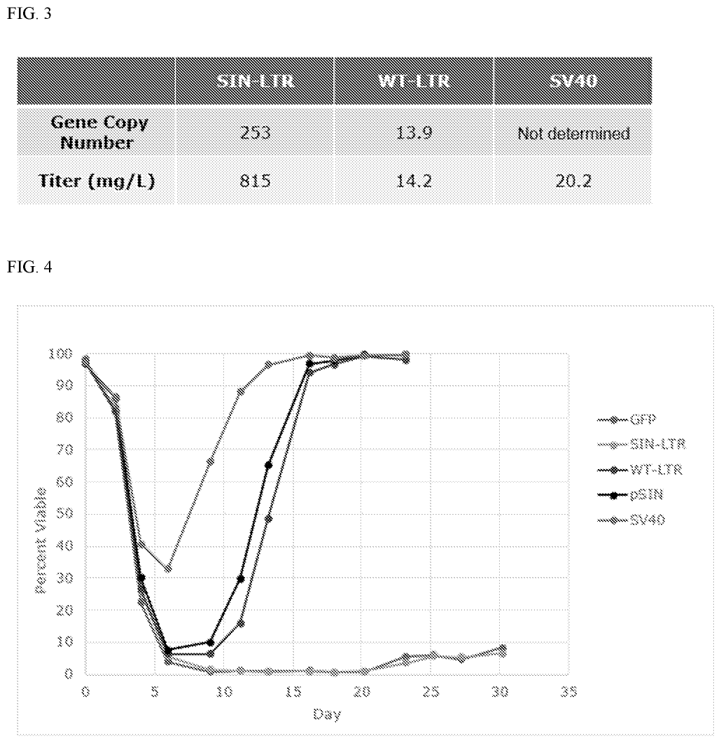

As displayed in FIG. 3, titer was dramatically higher is the SIN-LTR pool compared to the WT-LTR and SV40 pools. In contrast, gene copy numbers showed a similar trend. These data indicate that the SIN-LTR plasmid selects for higher copy number and insertion sites with higher activity.

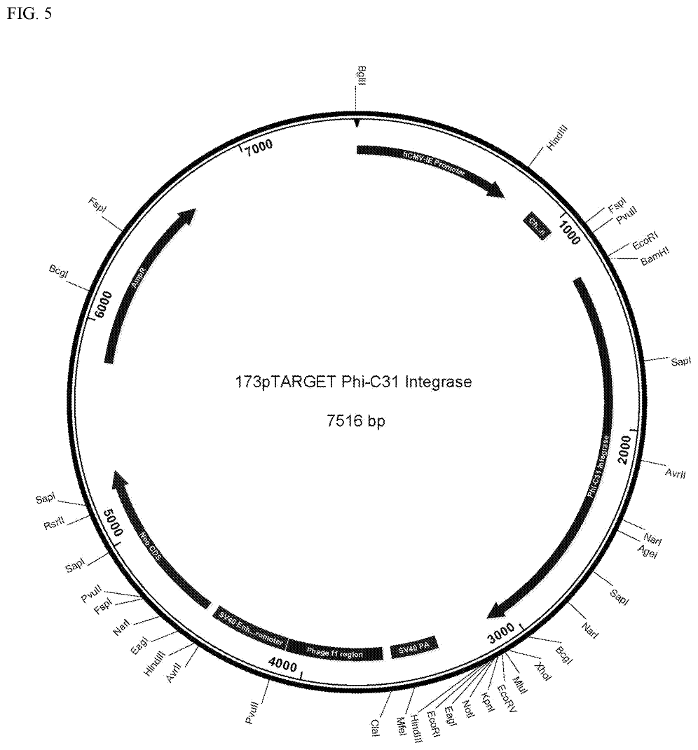

Surprisingly, in a separate experiment displayed in FIG. 4, pSIN pools had a recovery time similar to SV40 or WT-LTR pools. Therefore, promoter activity alone does not explain the differences in recovery time since pSIN has a very weak promoter but still recovered quickly. Other elements in the SIN-LTR plasmid must be responsible for the stronger selection pressure. While not being limited to any particular mechanism of action, it is contemplated that the combination of the weak promoter and long transcript, which also contains a second open reading frame, may affect the transcriptional or translational efficiency of the GS. Likewise, without being limited to any particular mechanism, the known presence of a weak Kozak sequence in the EPR could lead to aberrant translation, reducing the translation efficiency of the GS protein.

### Example 2

The GPEx Boost concepts may also be used in combination with other non-viral gene insertion technologies such as transposase, recombinase, integrase or CRISPR gene insertion. GPEx technology can be used to place many copies of the recognition sequence for the non-viral insertion technology at highly active sites throughout the genome. The resulting “Dock” cell line can then be transiently co-transfected with a plasmid expressing the transposase, recombinase, integrase, or Cas9 in combination with a transgene plasmid that contains the cognate recognition sequence, the GS selectable marker, and the gene product to be expressed. The transposase, recombinase, integrase, or Cas9 will mediate the insertion of a part or all of the transgene plasmid into the Dock sites. The resulting cell line will have multiple copies of the transgene plasmid inserted into highly active dock sites throughout the genome. Some examples of technologies/enzymes that can be used include piggyback transposase, sleeping beauty transposase, Mos1 transposase, To12 transposase, Leapin transposase, Lambda recombinase, FLP/FRT, Cre/Lox, MMLV integrase, Rep 78 integrase, Bxb1 integrase, and various types of CRISPR. We first tested this concept using the PhiC31 Integrase system in combination with GPEx technology.

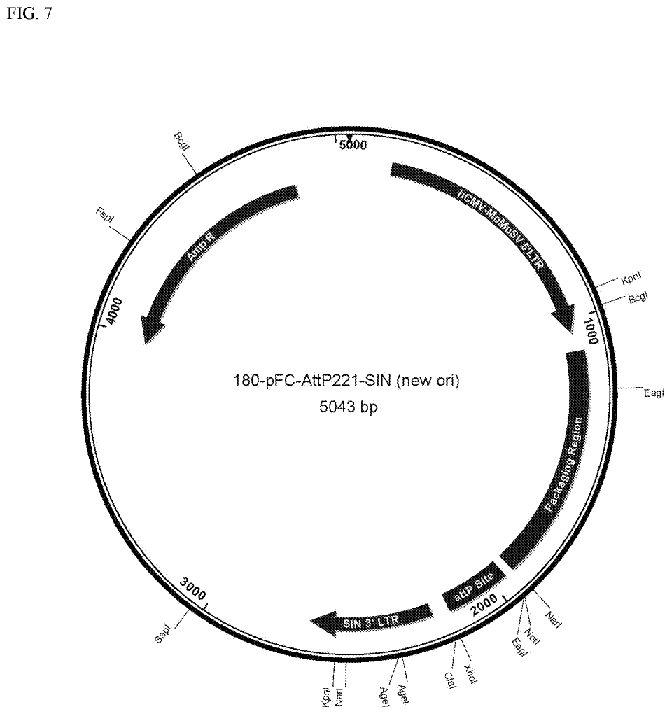

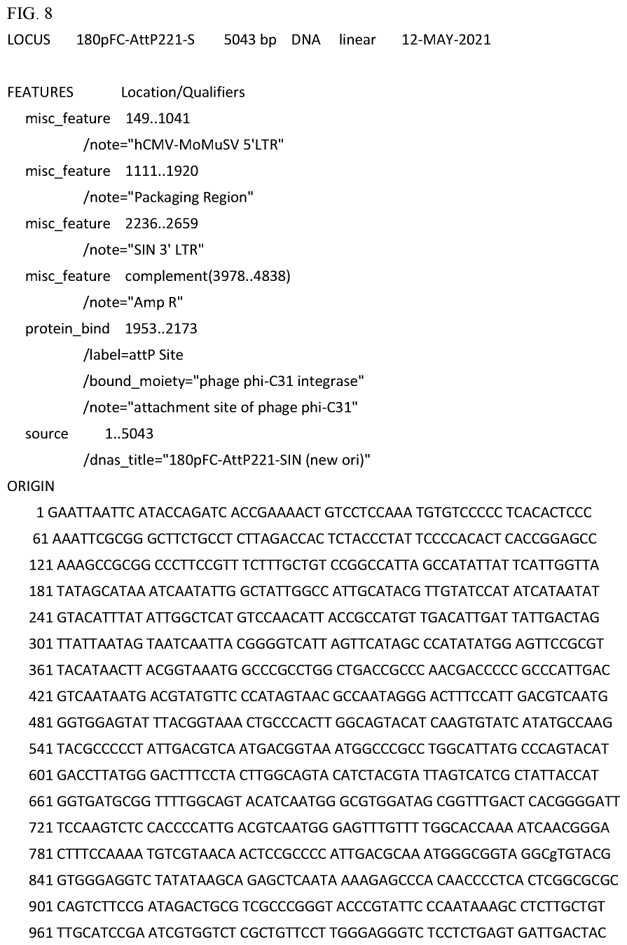

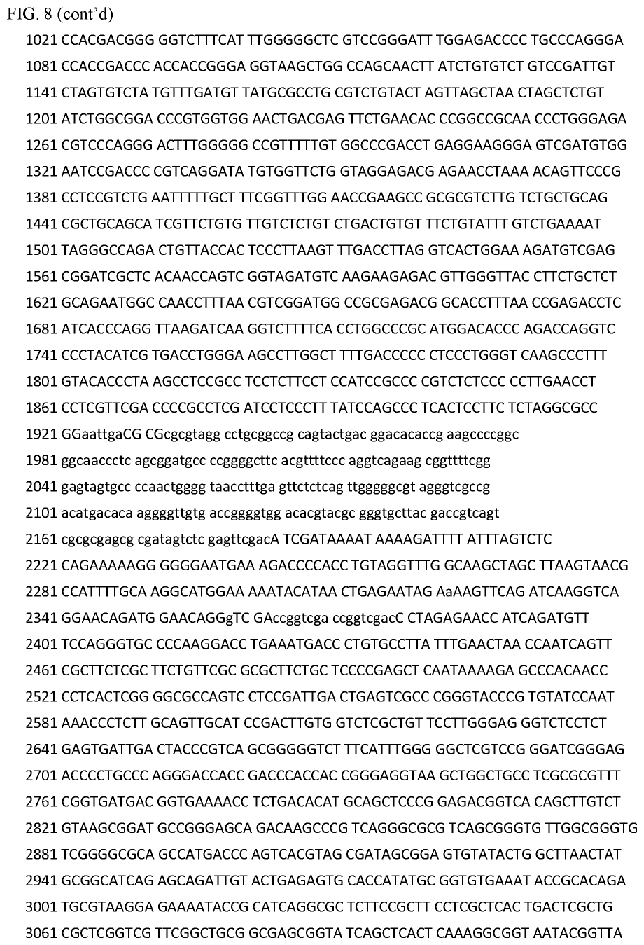

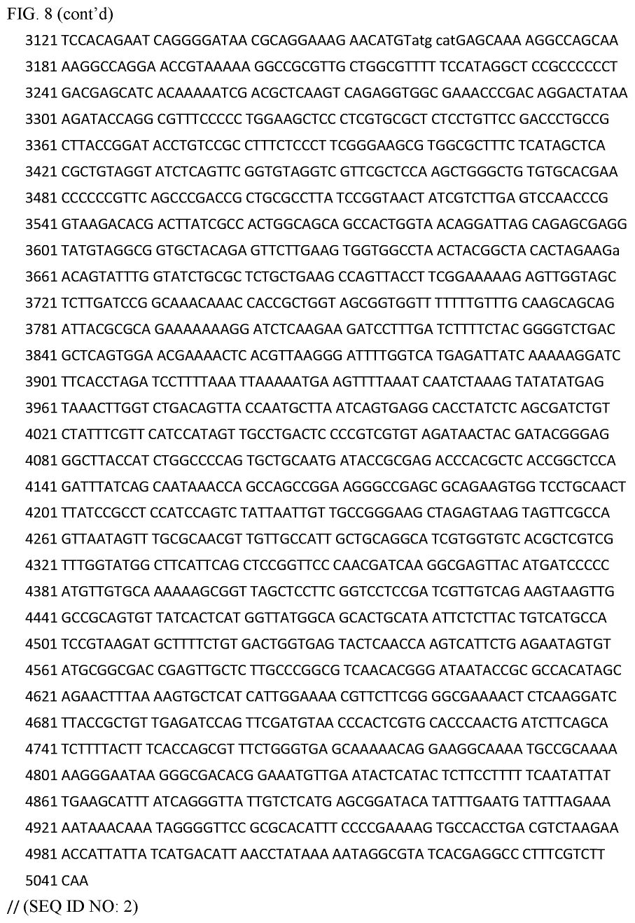

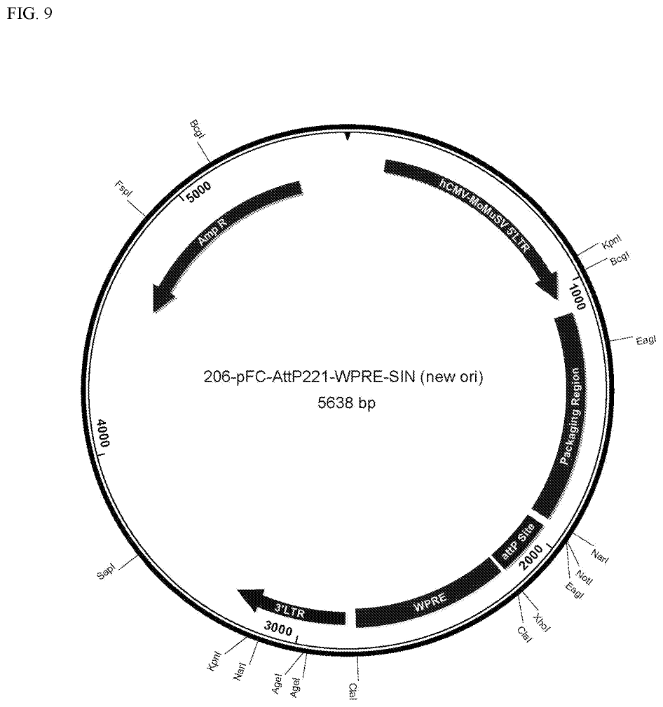

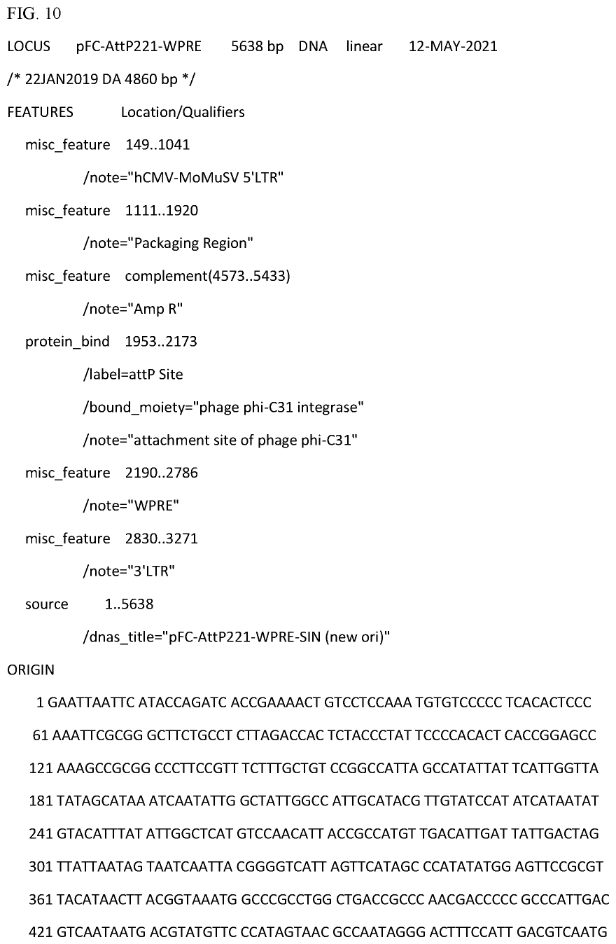

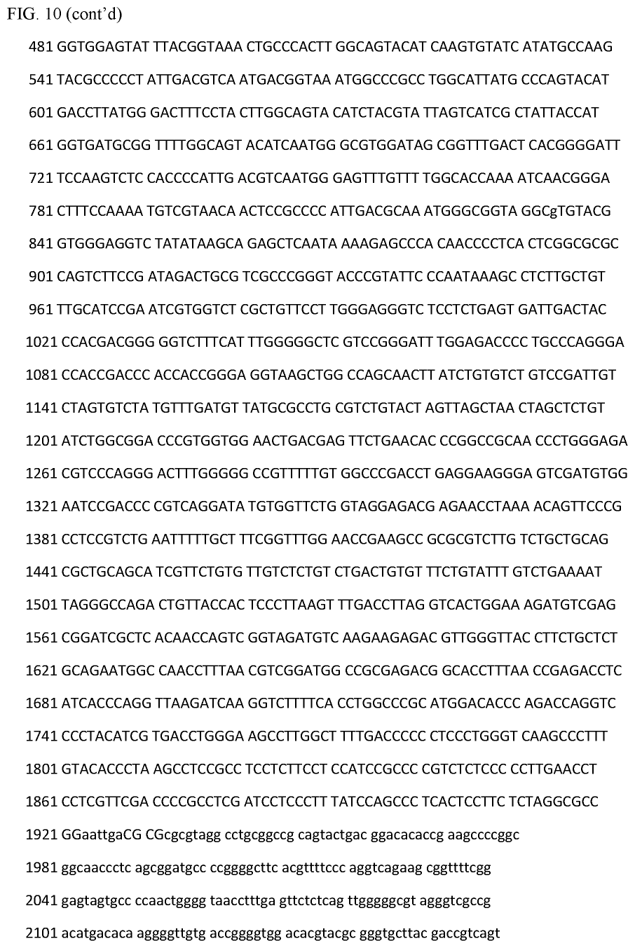

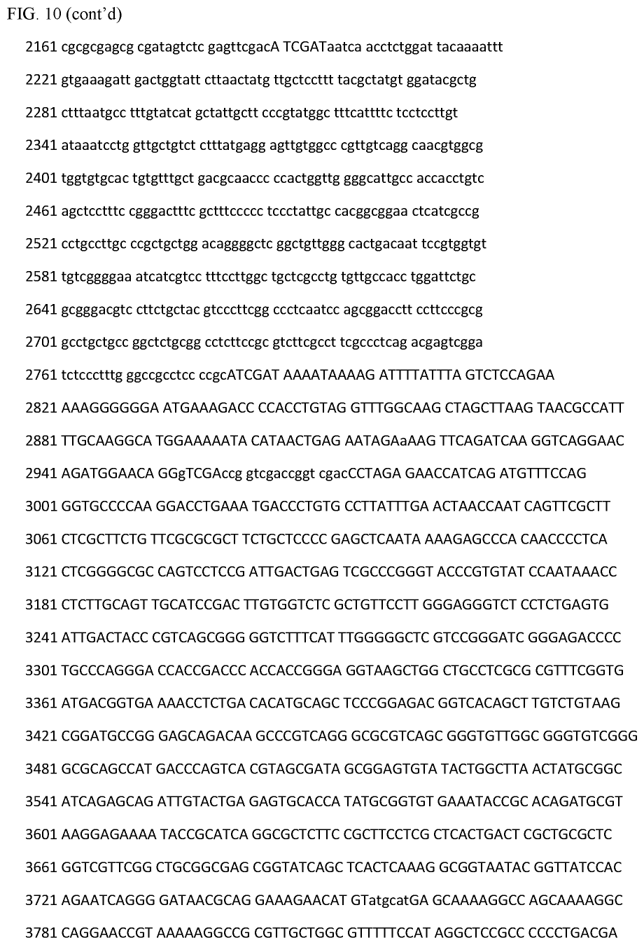

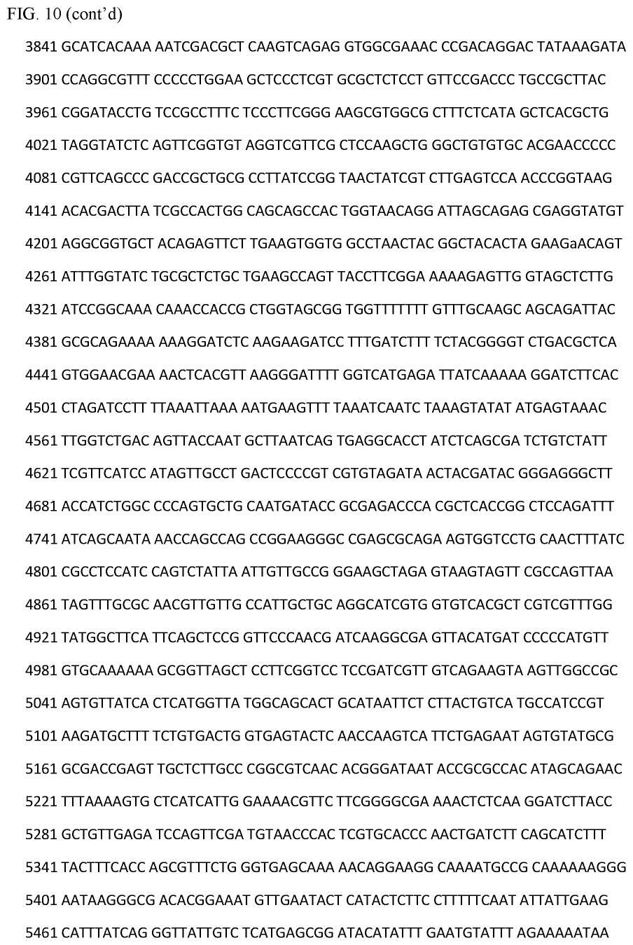

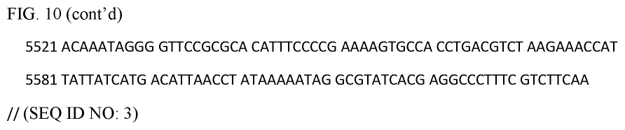

Retrovector Production and Transduction to Create the Dock Parental Cell Line: The Dock construct, FIGS. 7 and 8, was introduced into a HEK 293 cell line that constitutively produces the MLV gag, pro, and pol proteins. An envelope containing expression plasmid was also co-transfected with the each of the gene constructs. The co-transfection resulted in the production of replication incompetent high titer retrovector that was concentrated by ultracentrifugation and used for cell transductions of the CHOZN Chinese Hamster Ovary parental cell line (1,2). 5 sequential rounds of transduction were performed, and cells were routinely maintained media supplemented with 6 mM glutamine. A second pooled dock cell line was also produced successfully using the same methods. This was using the slightly different dock gene construct shown in FIGS. 9 and 10.

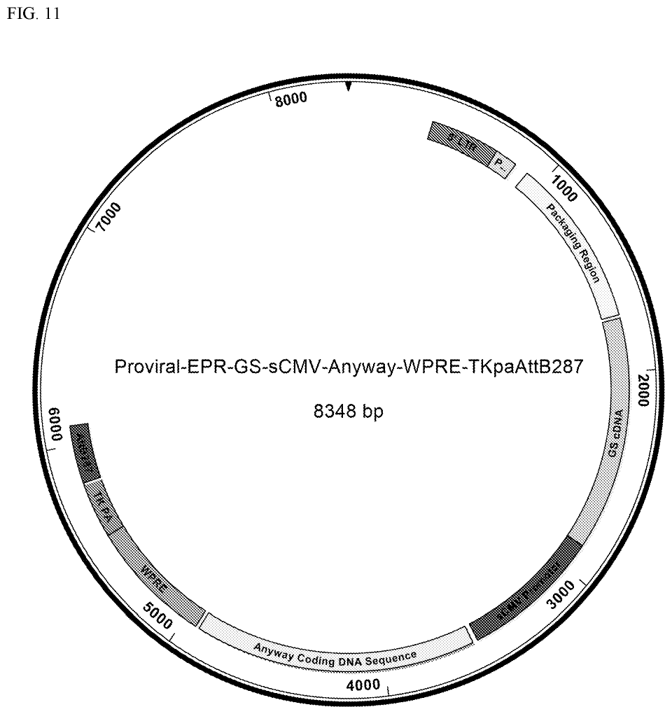

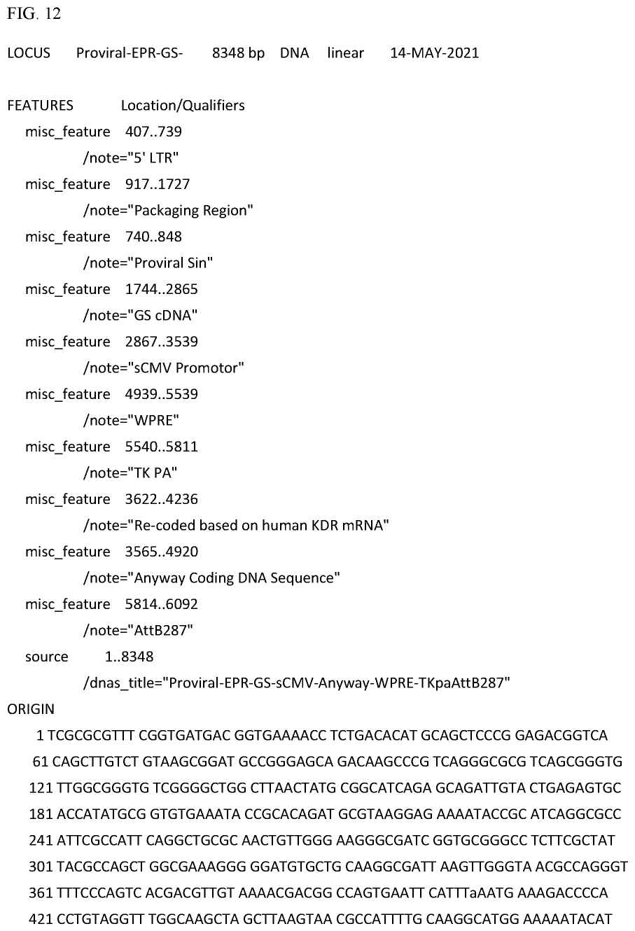

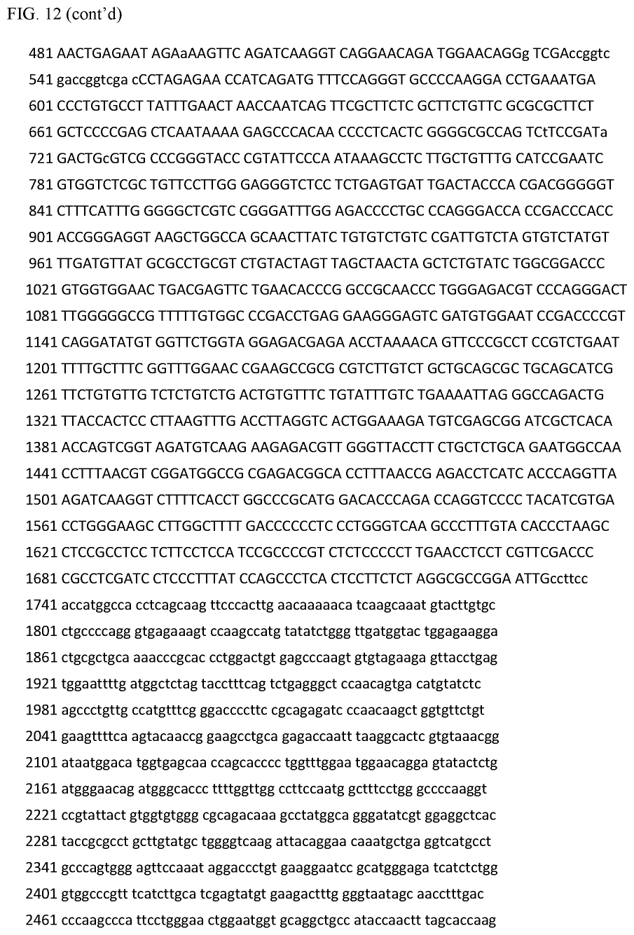

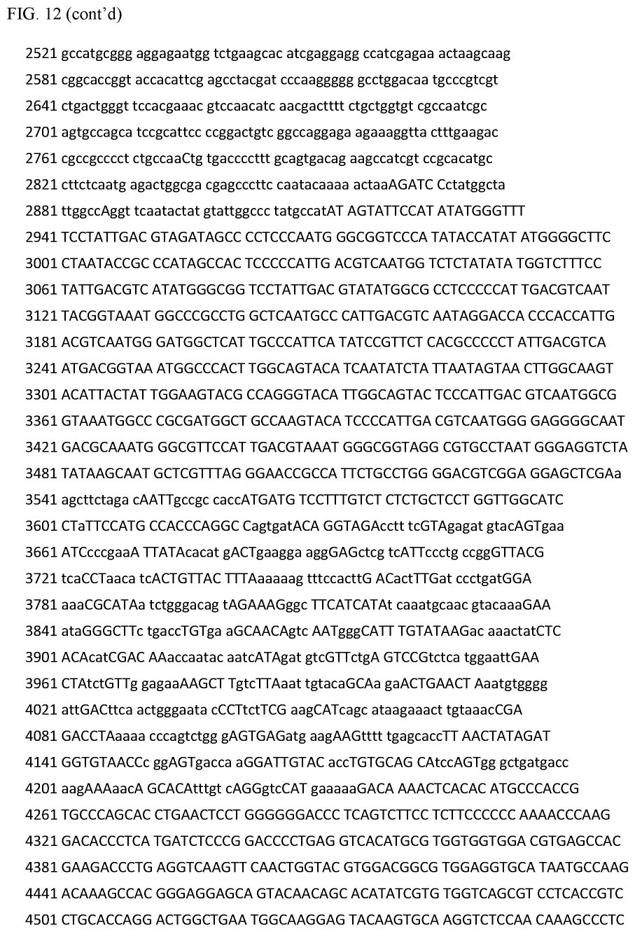

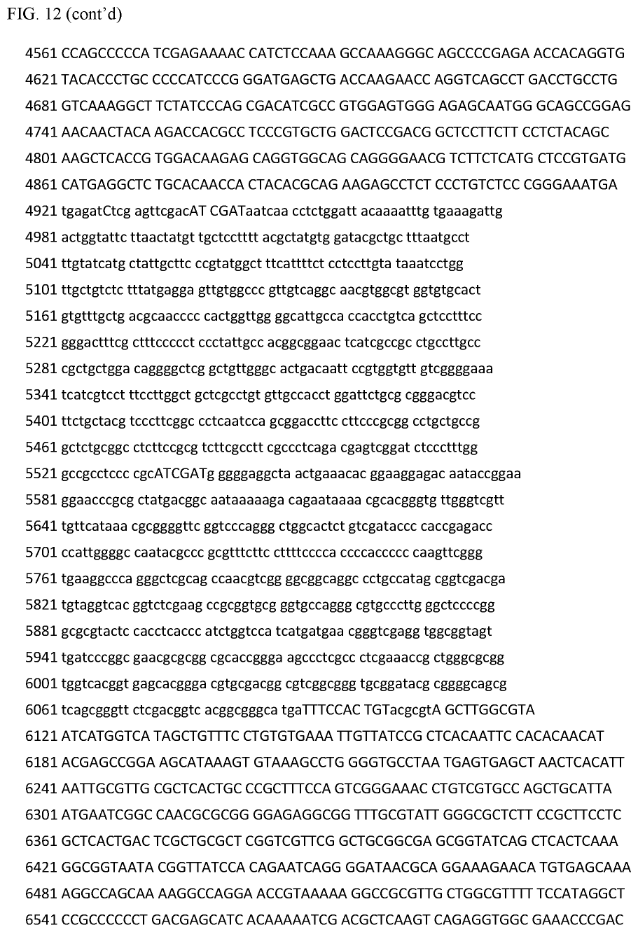

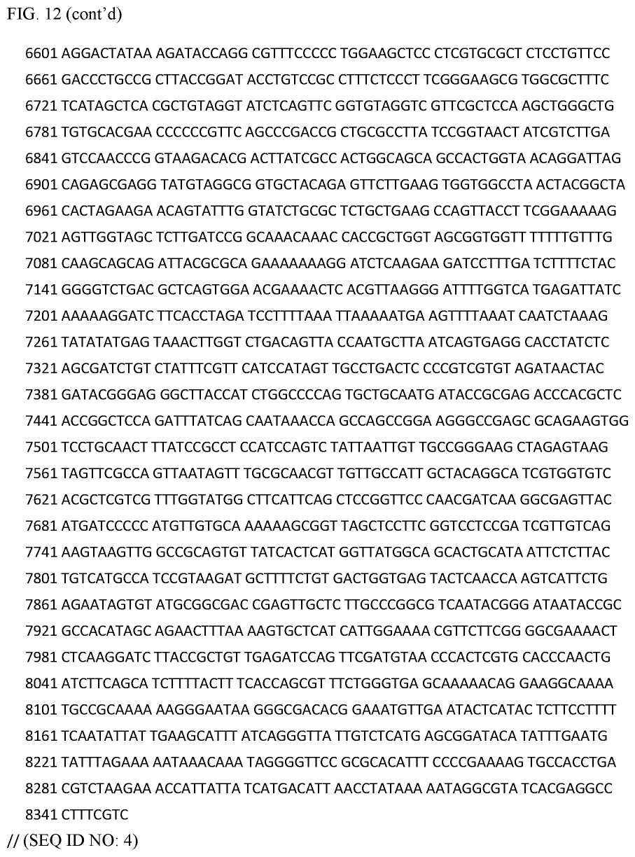

Transfection and Selection of Dock Pooled Cell Line: 1.5 million cells were incubated with a precomplexed mixture containing 1 ug (total) of plasmid transgene and Integrase DNA (FIGS. 11, 12, 5 and 6 respectively) and 4 microliters of ExpiFectamine CHO™ (ThermoFisher Scientific) in a final volume of 250 microliters. Pooled cell lines were allowed to recover in the presence of media supplemented with 6 mM glutamine until viability returned to greater than 95%. Cells were then transferred to media lacking glutamine. Viability was monitored and media was replaced weekly until the resulting selected cell pools returned to greater than 95% viability.

Quantification of Recombination: Genomic DNA from 3 million cells was isolated using a Qiagen DNEasy kit. AttR is the result of recombination between attP and attB. Quantitative Polymerase Chain Reaction (QPCR) using sybr-green dye was performed to quantify attR in the cells using a forward primer in the attP sequence in the dock and a reverse primer in the attB sequence in the transgene plasmid. Amplification using this primer pair will only detect the transgene plasmid when it is recombined into the dock and not free, randomly integrated, or pseudo-attP integrated transgene plasmid. Similarly, this primer pair will not detect unrecombined (empty) dock sequence. The number of PCR cycles needed to cross a fluorescence intensity threshold (Ct value) was determined for this primer set as well as a primer set for an internal CHO reference gene. Gene Copy Indexes (GCIs) were calculated by subtracting the Ct value of the reference gene from the Ct value of the attR primer set. Note that GCI values are a logarithmic, not linear, in nature such that a change of 1 unit at the low end of the scale, ex from GCI=1 to GCI=3, represents a difference of only a few copies whereas a change in one unit at the higher end of the scale, ex from GCI=6 to GCI=7 can represent a difference of numerous copies. In some cases, a plasmid containing the desired amplicons and of known concentration was also subjected to QPCR and this data was subjected to linear regression analysis to more precisely determine the number of copies present.

### Results

Docks containing the PhiC31 attP recognition sequence, FIG. 7+8, were placed throughout the genome of CHOZN cells with 5 sequential rounds of transduction using GPEx technology and the resulting cell pool contained approximately 36 Dock copies per cell on average. This Dock cell pool was co-transfected with Transgene-Promoter-Anyway and Integrase plasmids (FIGS. 11, 12, 5 and 6 respectively) at ratios ranging from 1:50 to 1:1 as suggested published literature (Groth, 2000: Andreas, 2002: Farruggio, 2012). This Transgene-Promoter-Anyway plasmid contains the PhiC31 attB recognition sequence, the glutamine synthetase (GS) gene driven by weak proviral-SIN-LTR (Self-Inactivating Long Terminal Repeat) promoter, and an Fc fusion protein test product, Anyway, driven by a strong promoter. 3 days after transfection but before selection, QPCR was performed to quantify recombination but attR (the upstream product off recombination between attP and attB) levels were not detectable above background with gene copy indexes (GCIs) of approximately −10. When transfected cells were subjected to selection through Glutamine withdrawal, they did not recover after more than 25 days indicating they had not achieved sufficient levels of integration and GS expression.

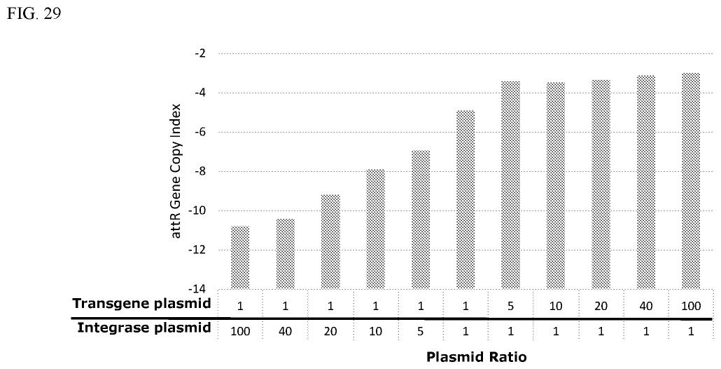

In an attempt to improve recombination frequency, we reasoned that the integrase to transgene plasmid ratios might be a critical parameter for efficient recombination. To explore this possibility, we co-transfected the Dock cell pool with a range of ratios of the Transgene-Promoter-Anyway and Integrase plasmid. 3 days after transfection but before selection, QPCR was performed to quantify recombination, FIG. 29. Ratios containing low Transgene:Integrase ratios (1:20-100) that are commonly used in the literature had attR GCIs near the background level of −10. Surprisingly, we found that high Transgene:Integrase ratios (5-100:1) had attR GCIs of −3 which is approximately 200-fold higher copy number than the lowest ratios.

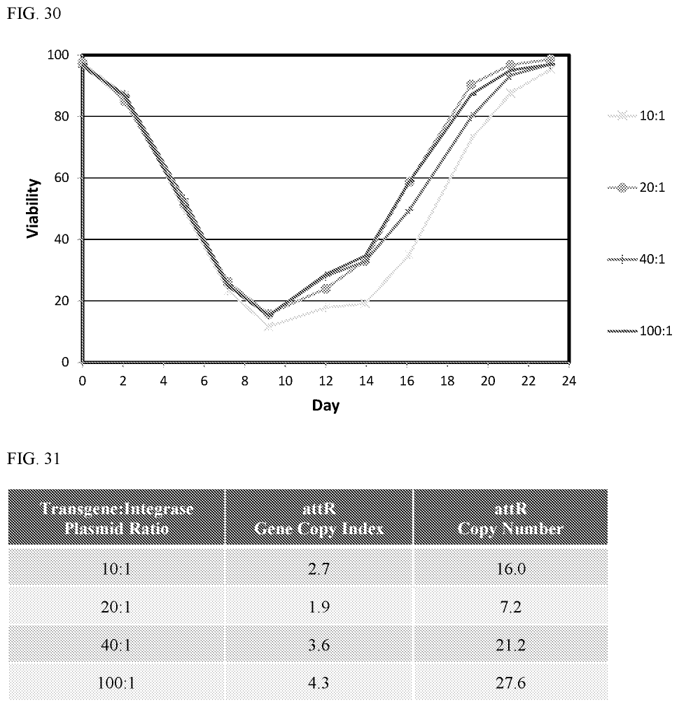

We then performed selection via glutamine withdrawal on the samples with the highest preselection attR GCIs, FIG. 30. These pools began to recover starting on day 9 of selection. After full recovery, we performed QPCR, FIG. 31, and found that these pools contained up to approximately 28 copies of transgene per cell. These data indicate that by using higher Transgene:Integrase ratios we were able to achieve efficient integration of up to an average of 28 transgenes per cell in a pool that contained approximately 36 docks on average. Further, this recombination was approximately 2 orders of magnitude higher than the level of recombination seen using lower ratios described in the literature.

### Example 3

After observing approximately 80% fill in a Dock pool containing about 36 copies per cell, we next sought to determine if we could increase the number of integrated Transgene plasmids further using a Dock pool that contained more than 36 docks. We also sought to determine if a Transgene plasmid that lacked a GS promoter could also be used in this system. Such a plasmid would only express GS, and thus contribute to resistance, if recombined into the Dock and not if randomly integrated or integrated into pseudo-attP sites

Retrovector Production and Transduction to Create the Dock Parental Cell Line: The Dock construct (FIGS. 7 and 8) was introduced into a HEK 293 cell line that constitutively produces the MLV gag, pro, and pol proteins. An envelope containing expression plasmid was also co-transfected with the each of the gene constructs. The co-transfection resulted in the production of replication incompetent high titer retrovector that was concentrated by ultracentrifugation and used for cell transductions of the CHOZN Chinese Hamster Ovary parental cell line (1,2). 9 sequential rounds of transduction were performed, and cells were routinely maintained in media supplemented with 6 mM glutamine.

Transfection and Selection of Dock Pooled Cell Line: 3 million cells were incubated with a precomplexed mixture containing 2 ug (total) of plasmid Transgene and Integrase plasmid DNA, and 8 microliters of ExpiFectamine CHO™ (ThermoFisher Scientific) in a final volume of 500 microliters. Pooled cell lines were allowed to recover in the presence of media supplemented with 6 mM glutamine until viability returned to greater than 95%. Cells were then transferred to media lacking glutamine. Viability was monitored, media was replaced weekly, and cells were subcultured until the resulting selected cell pools returned to greater than 95% viability.

Quantification of Recombination: Genomic DNA from 3 million cells was isolated using a Qiagen DNEasy kit. AttR is the result of recombination between attP and attB. Quantitative Polymerase Chain Reaction (QPCR) using sybr-green dye was performed to quantify attR in the cells using a forward primer in the attP sequence in the dock and a reverse primer in the attB sequence in the transgene. Amplification using this primer pair will only detect the transgene plasmid when it is recombined into the dock and not free, randomly integrated, or pseudo-attP integrated transgene plasmid. Similarly, this primer pair will not detect unrecombined (empty) dock sequences. The number of PCR cycles needed to cross a fluorescence intensity threshold (Ct value) was determined for this primer set as well as a primer set for an internal CHO reference gene. Gene Copy Indexes (GCIs) were calculated by subtracting the Ct value of the reference gene from the Ct value of the attR primer set. Note that GCI values are a logarithmic, not linear, in nature such that a change of 1 unit at the low end of the scale, ex from GCI=1 to GCI=3, represents a difference of only a few copies whereas a change in one unit at the higher end of the scale, ex from GCI=6 to GCI=7 can represent a difference of numerous copies. In some cases, a plasmid containing the desired amplicons and of known concentration was also subjected to QPCR and this data was subjected to linear regression analysis to more precisely determine the number of copies present.

### Results

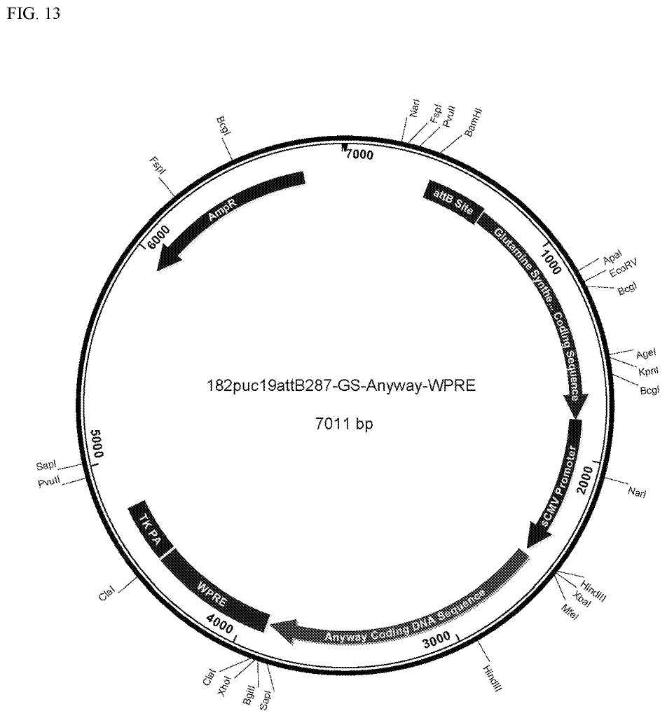

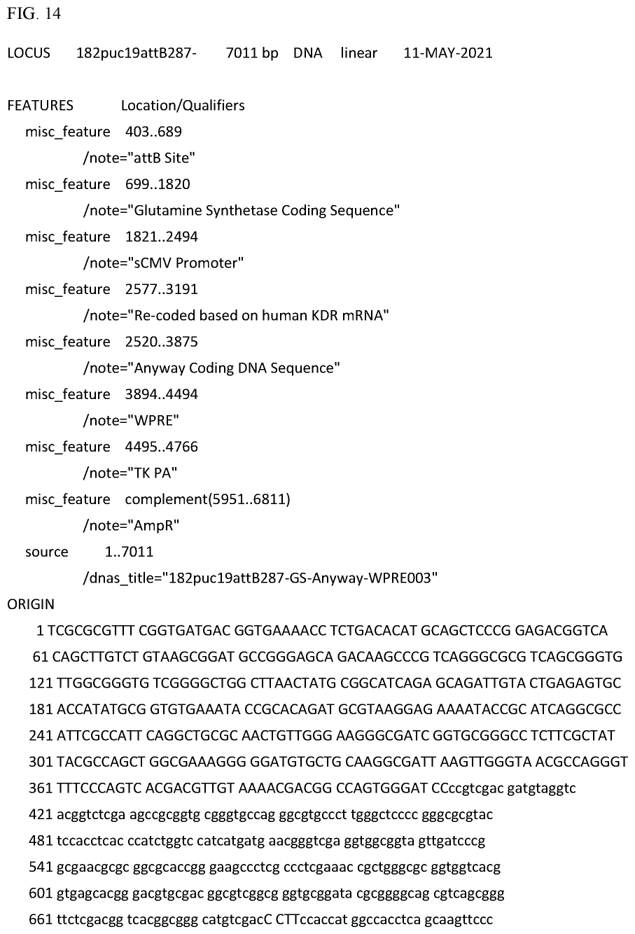

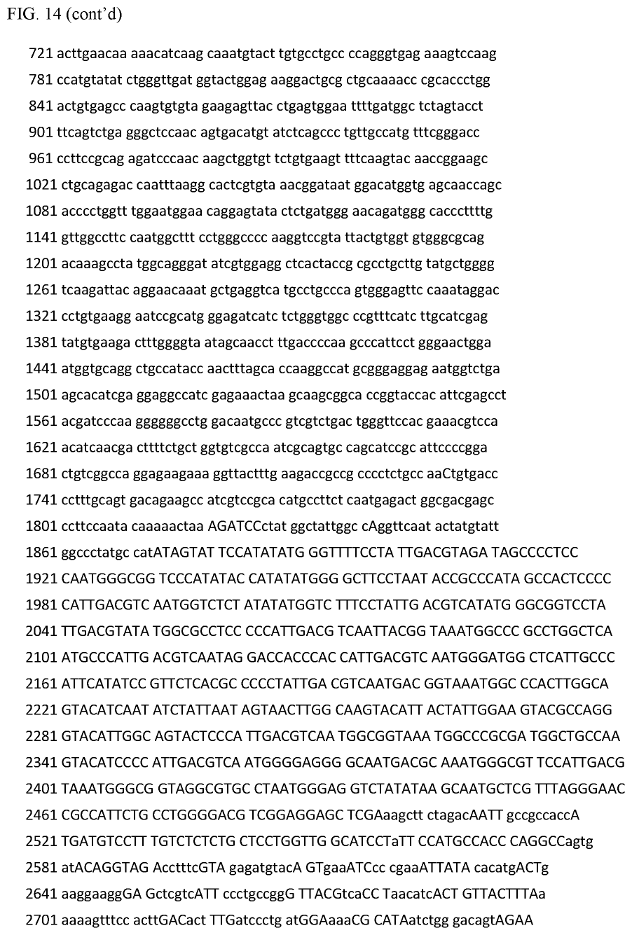

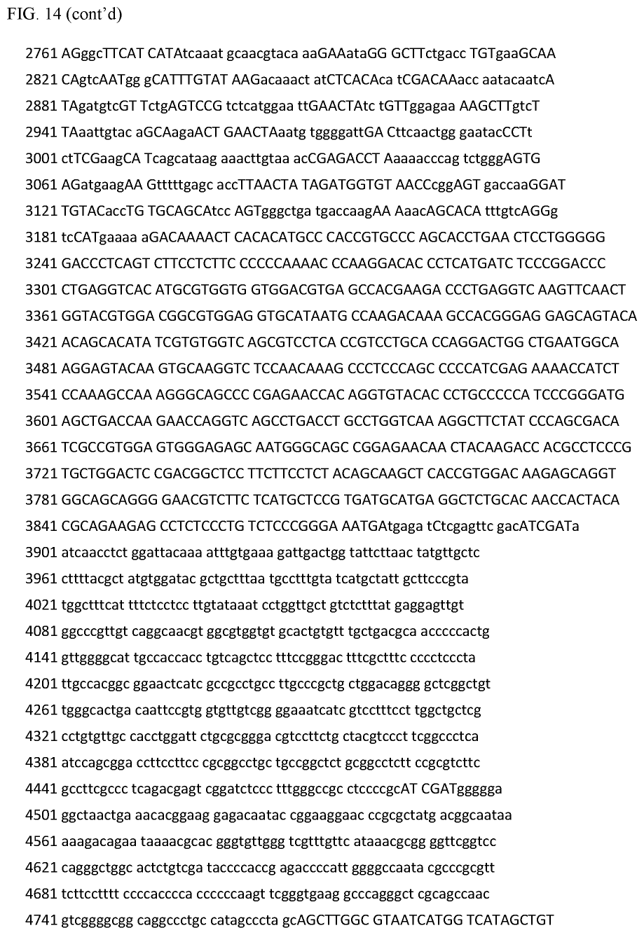

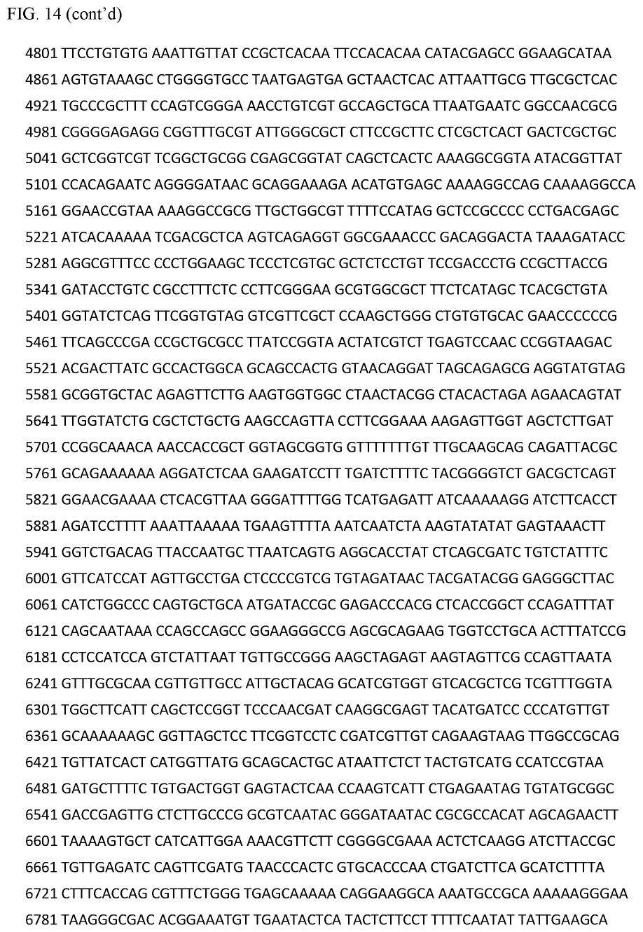

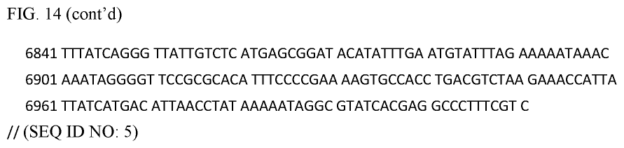

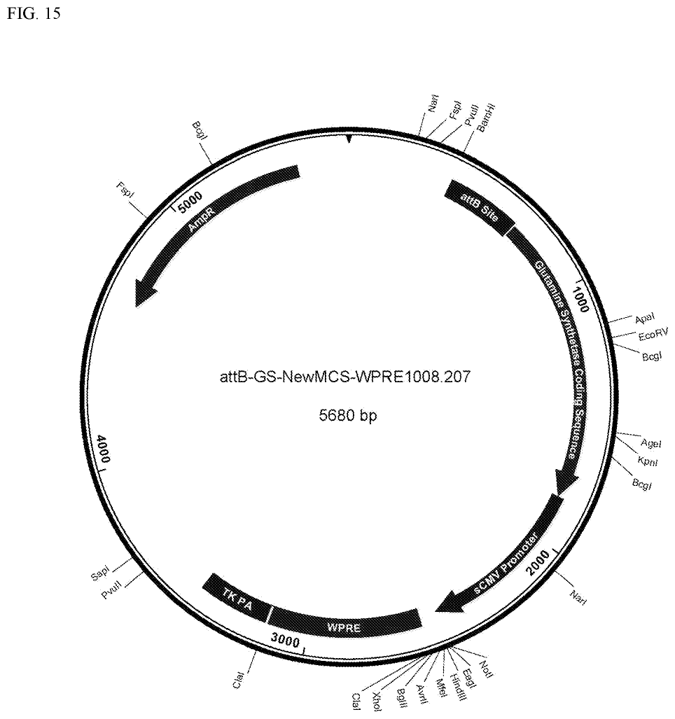

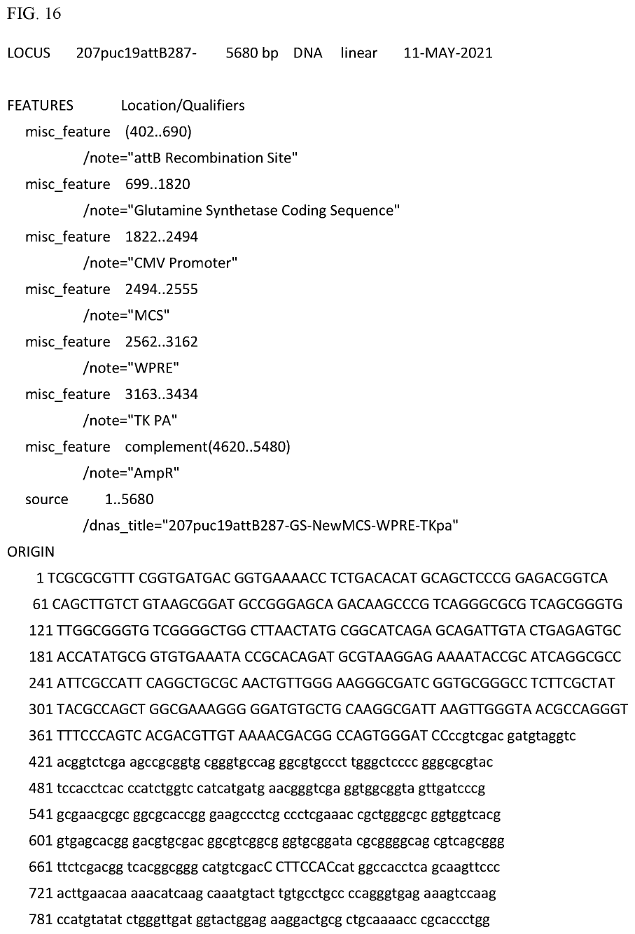

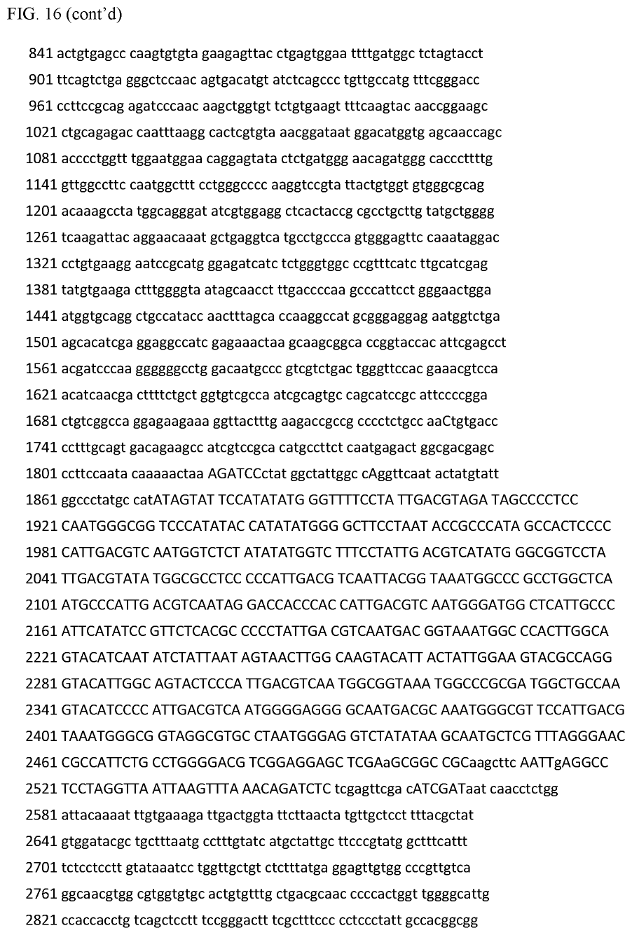

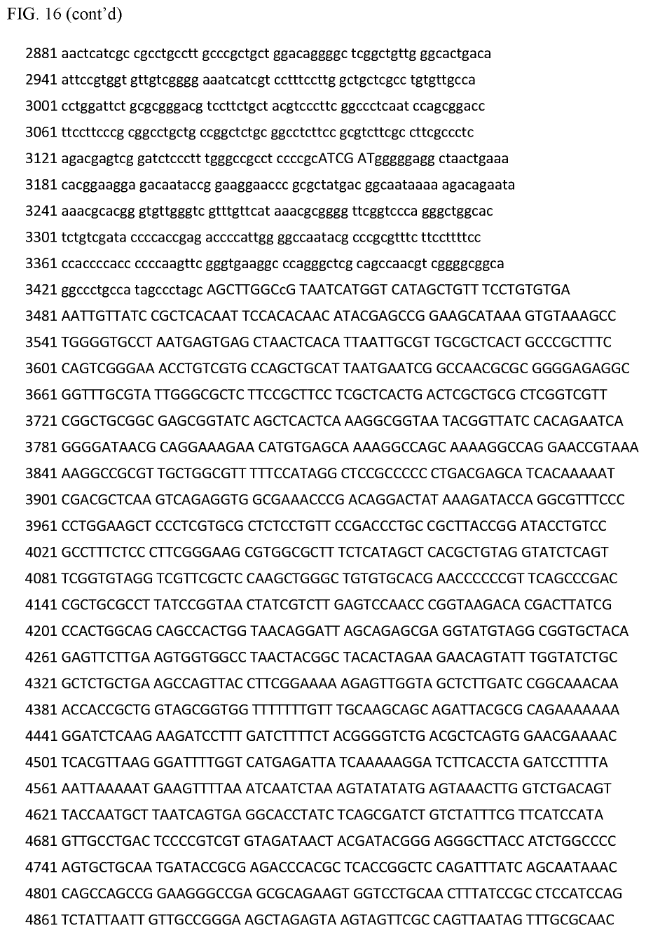

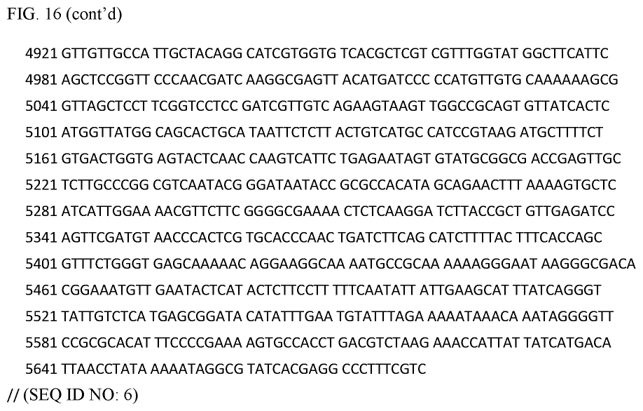

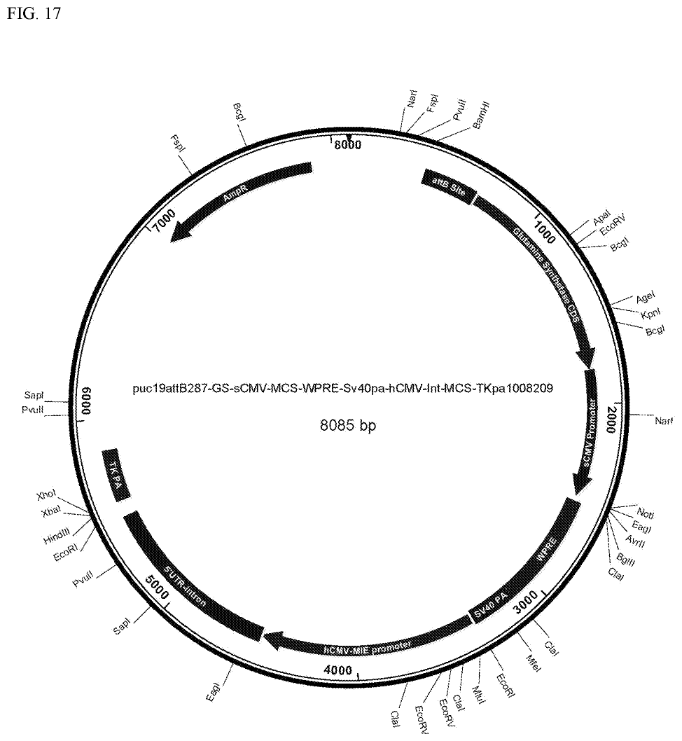

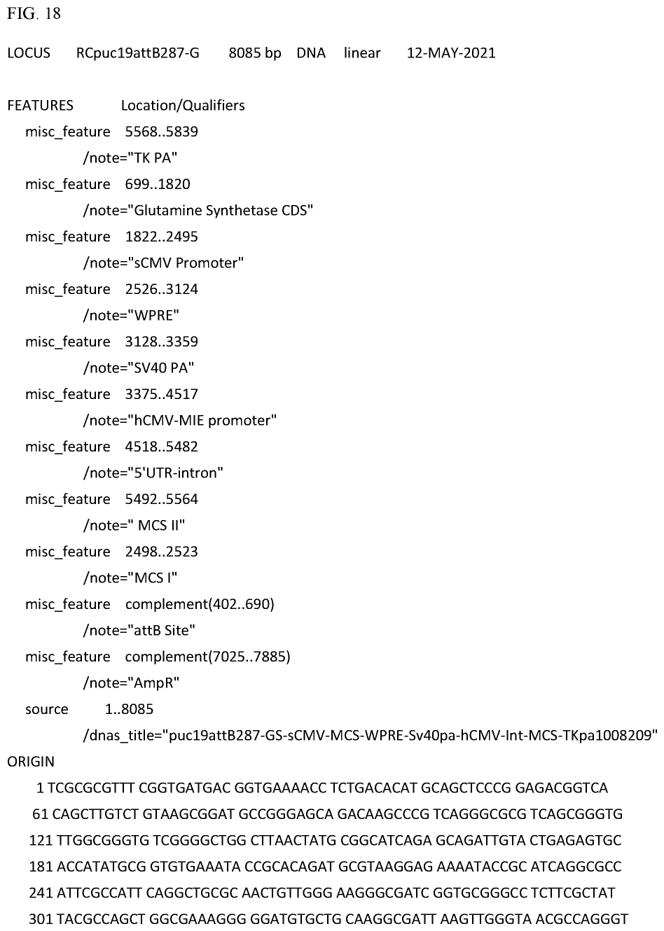

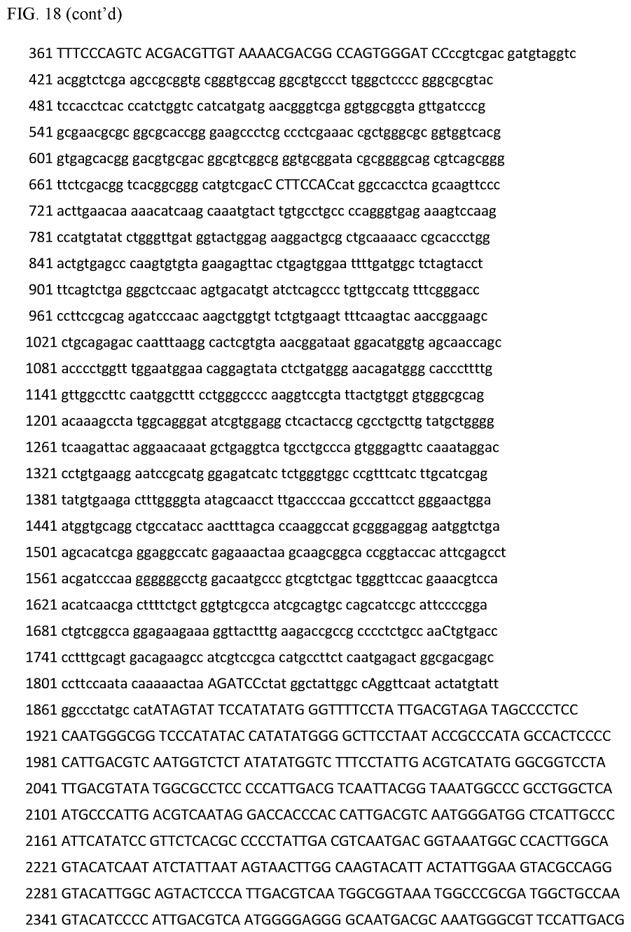

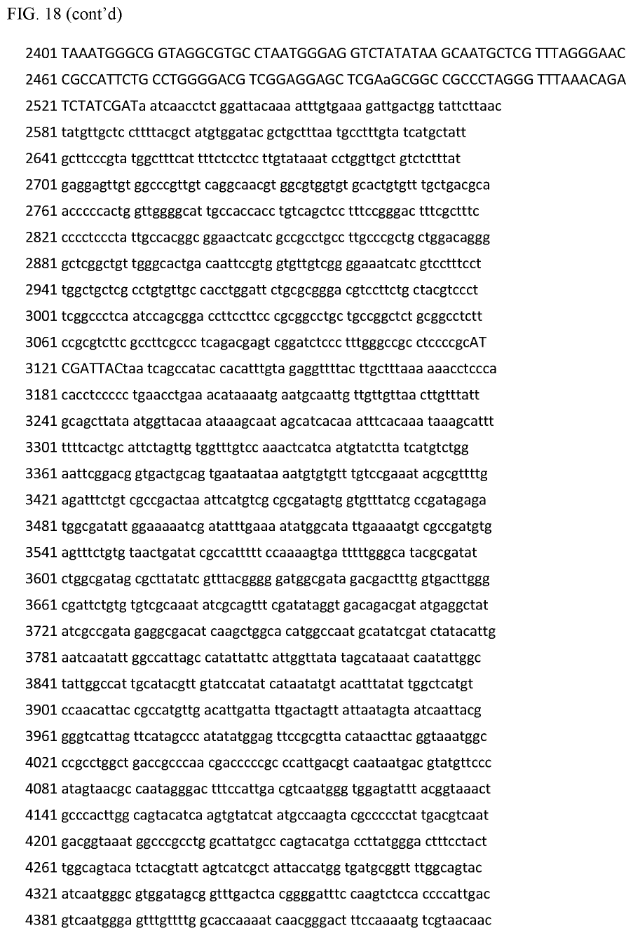

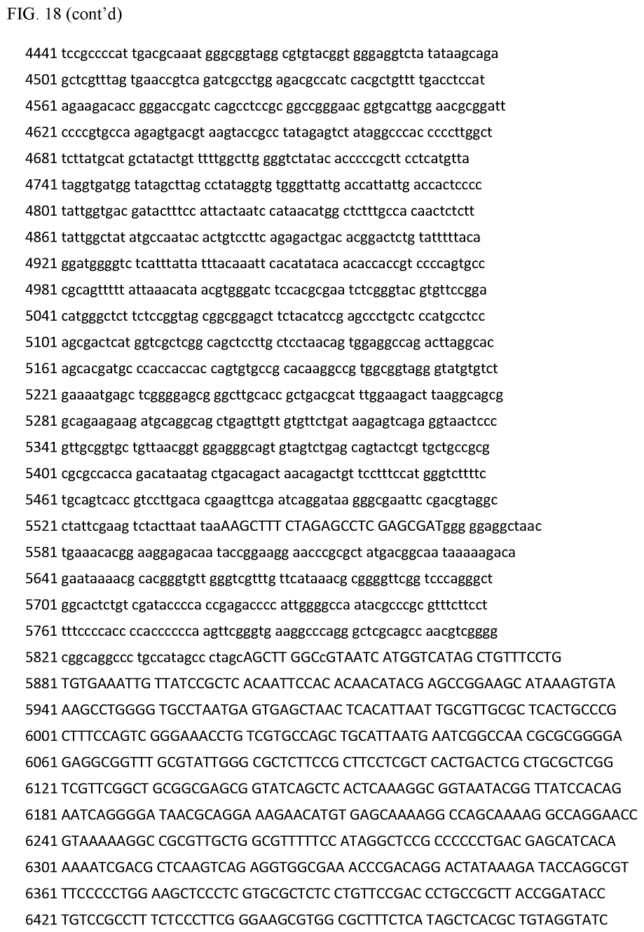

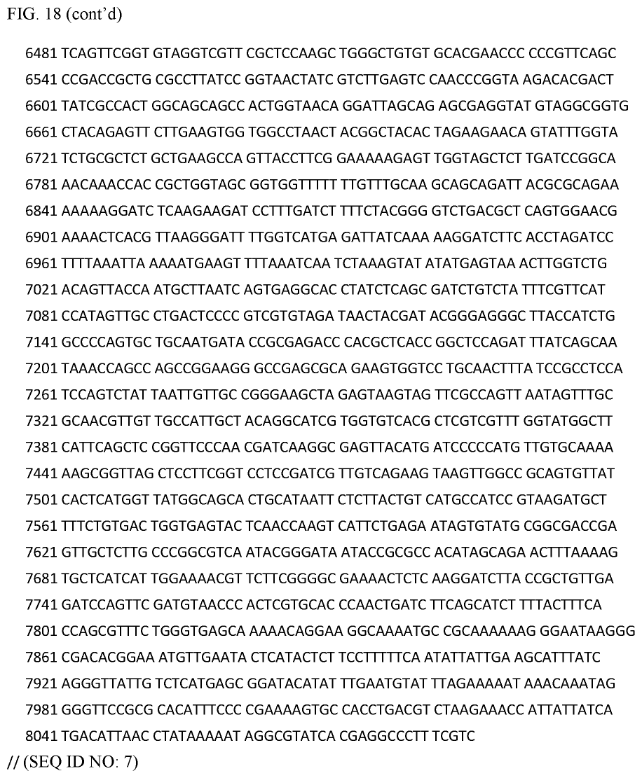

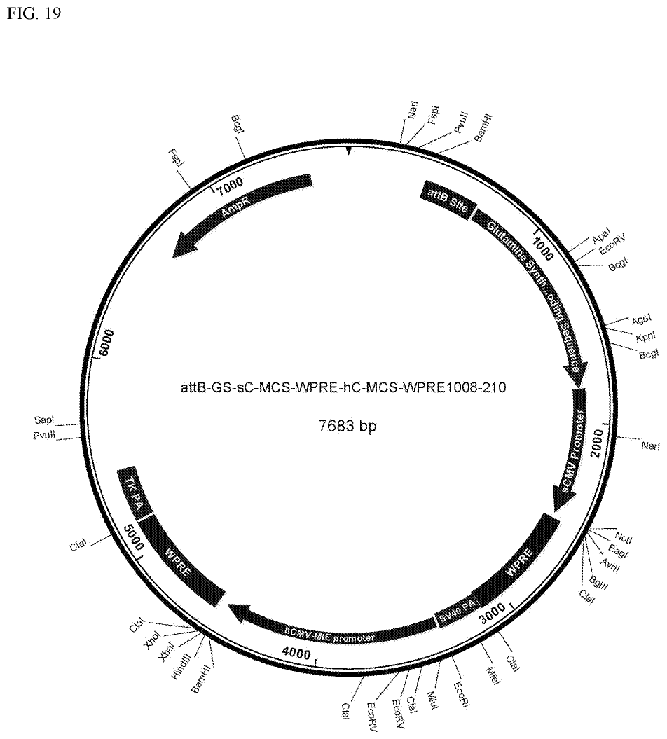

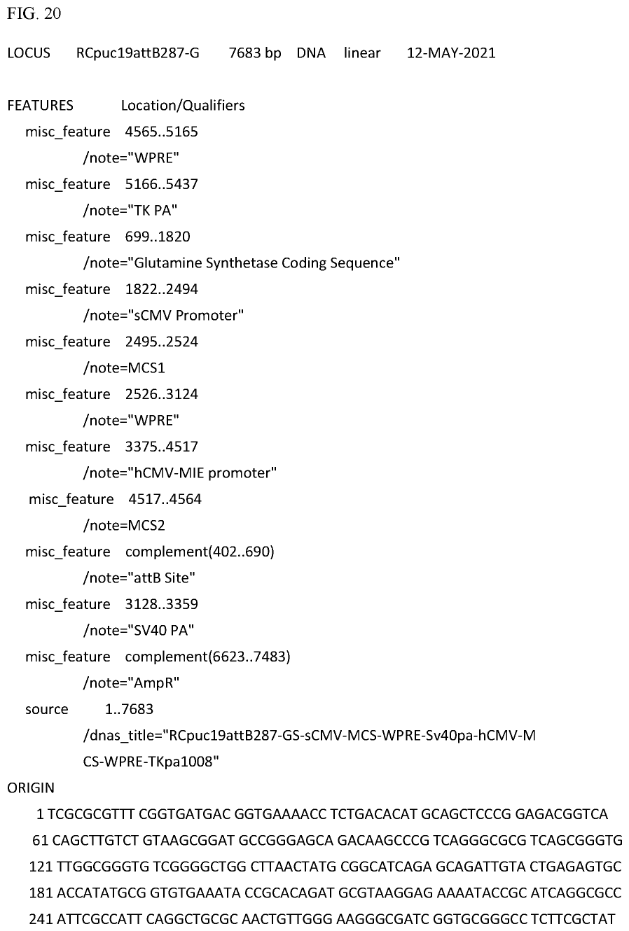

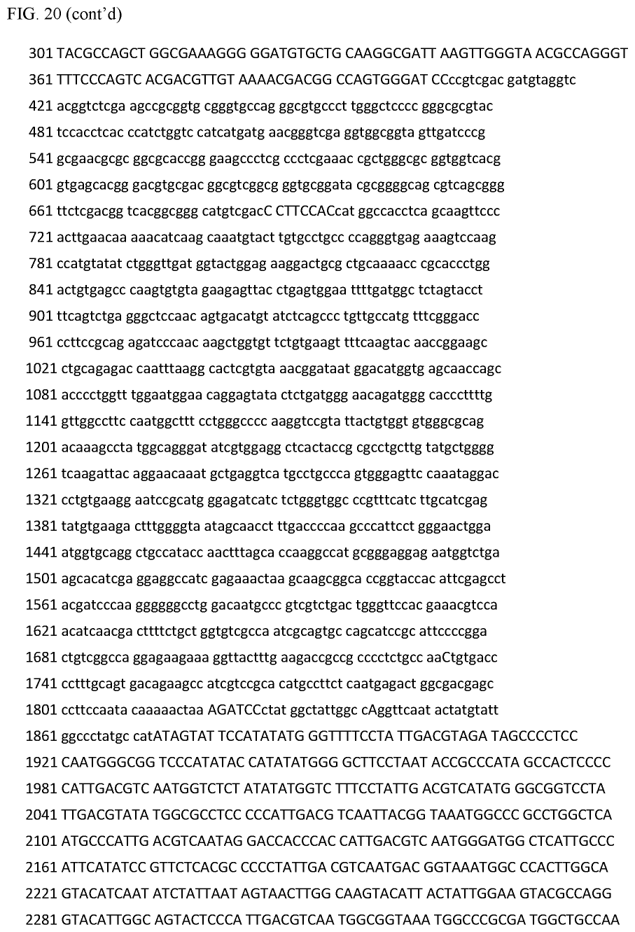

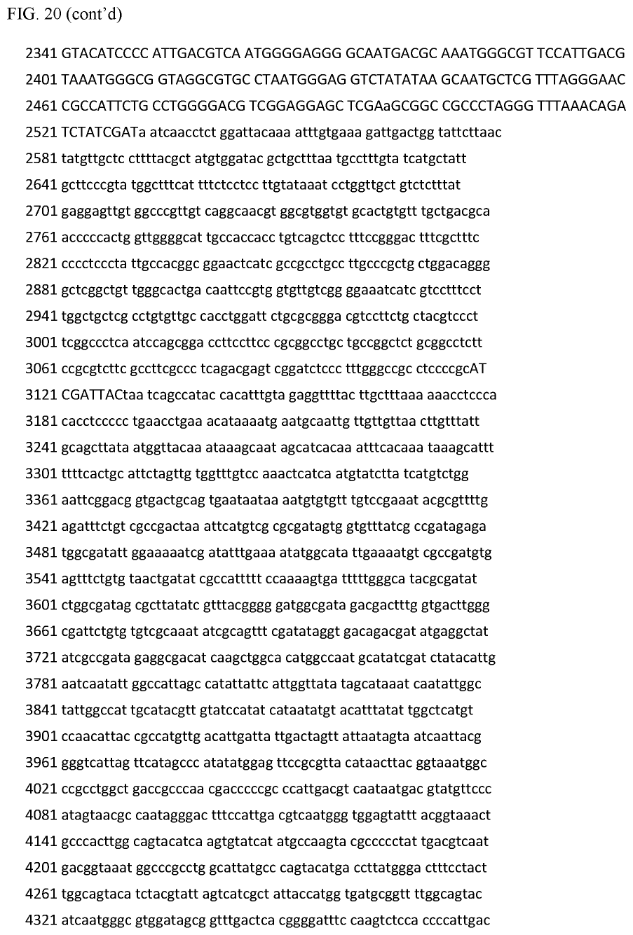

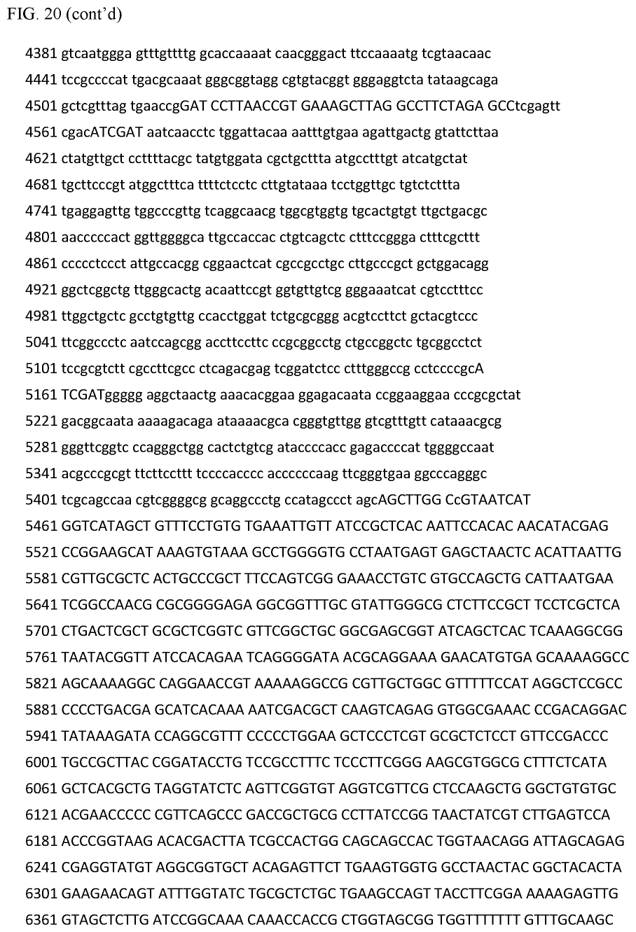

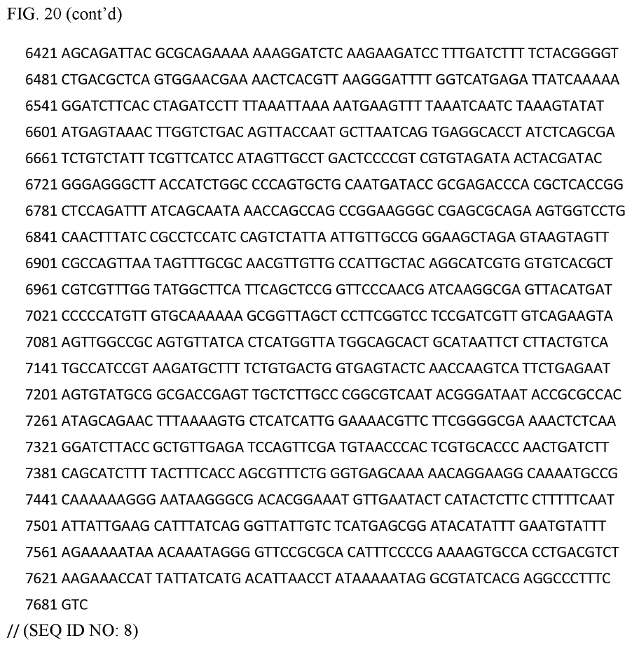

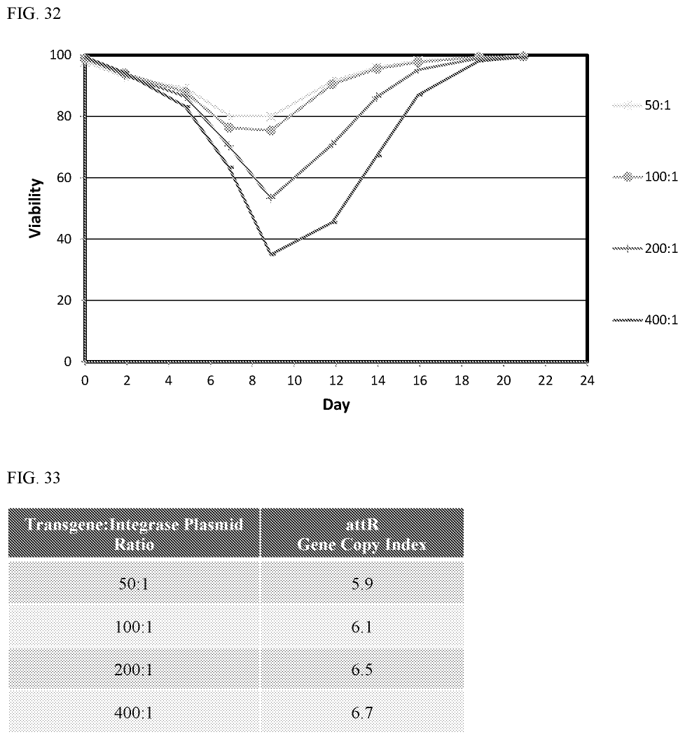

Docks containing the PhiC31 attP recognition sequence were placed throughout the genome of CHOZN cells with 9 sequential rounds of transduction using GPEx technology and the resulting cell pool had an EPR GCI of 6.7 and contained approximately 135 Dock copies per cell on average. This Dock cell pool was co-transfected with Transgene-Anyway and Integrase plasmids (FIGS. 13, 14, 5 and 6, respectively) at ratios ranging from 50:1 to 400:1 followed by selection via Glutamine withdrawal. Nadir (minimum) viability, FIG. 32, was higher than in the Dock line containing approximately 36 copies per cell. AttR GCIs, FIG. 33, were also higher than in the approximately 36 copy Dock cell line and increased with higher Transgene:Integrase ratios suggesting that even further improvement in the number of integrated transgenes might be possible at higher Transgene:Integrase ratios and higher Dock numbers. Additionally, we also demonstrated robust recovery from selection using the Transgene-Anyway plasmid, FIGS. 13 and 14, which lacks a promoter for GS and thus must rely on the weak, SIN-LTR promoter in the Dock.

### Example 4

Having improved integrated Transgene numbers using more Dock sites and higher Transgene:Integrase ratios, we next sought to determine if we could increase the number of integrated Transgene plasmids even further by isolating a higher Dock copy number clone from the 135 copy Dock pool and by testing even higher Transgene:Integrase plasmid ratios. We also sought to determine if larger plasmid sizes could be inserted with this technology.

Cloning of Dock Parental Cell Line: The Dock cell pool made from 9 sequential rounds of transduction was cloned using the Berkeley Lights, Beacon instrument. Clones were expanded, screened by QPCR and the clone with the highest number of dock insertions was selected.

Transfection and Selection of Dock Pooled Cell Line: 3 million cells were incubated with a precomplexed mixture containing 2 ug (total) of plasmid Transgene and Integrase DNA and 8 microliters of ExpiFectamine CHO™ (ThermoFisher Scientific) in a final volume of 500 microliters. Pooled cell lines were allowed to recover in the presence of media supplemented with 6 mM glutamine until viability returned to greater than 95%. Cells were then transferred to media lacking glutamine. Viability was monitored and media was replaced weekly until the resulting selected cell pools returned to greater than 95% viability.

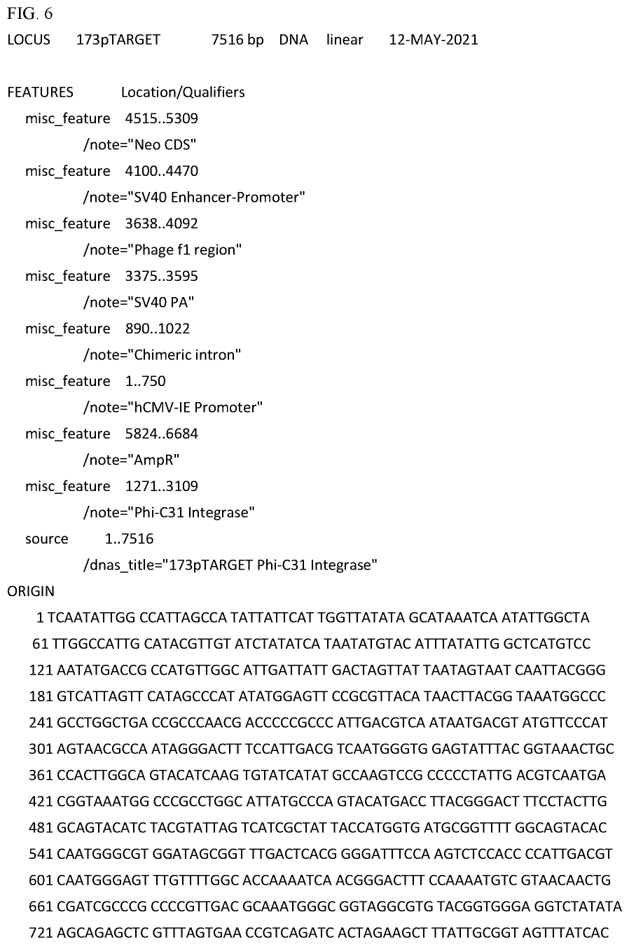

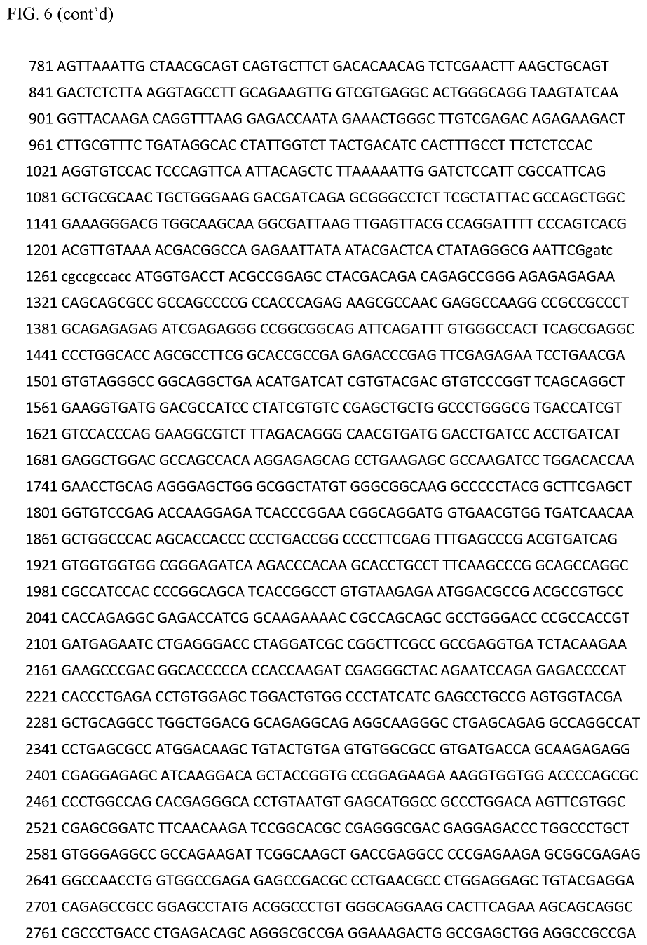

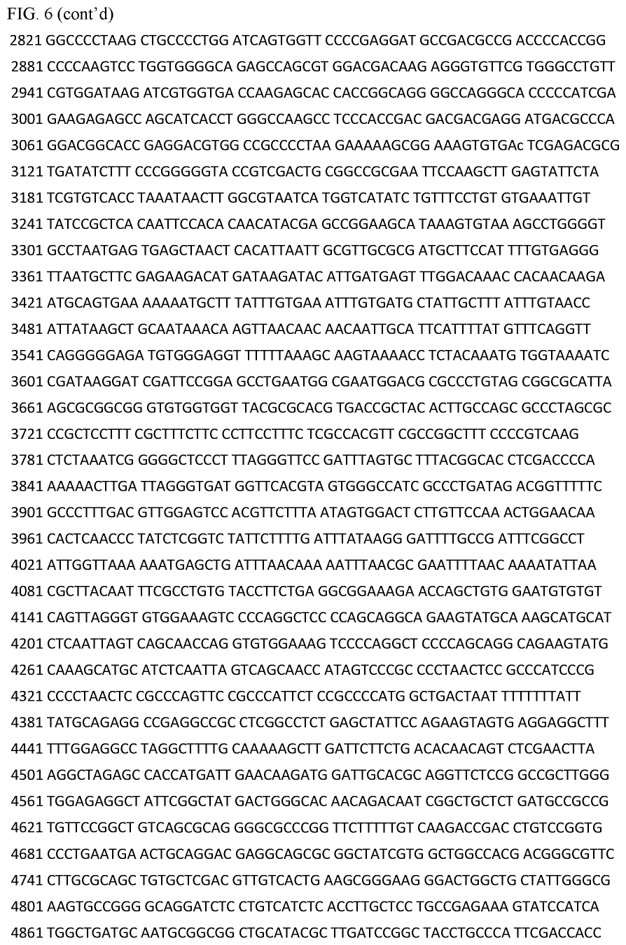

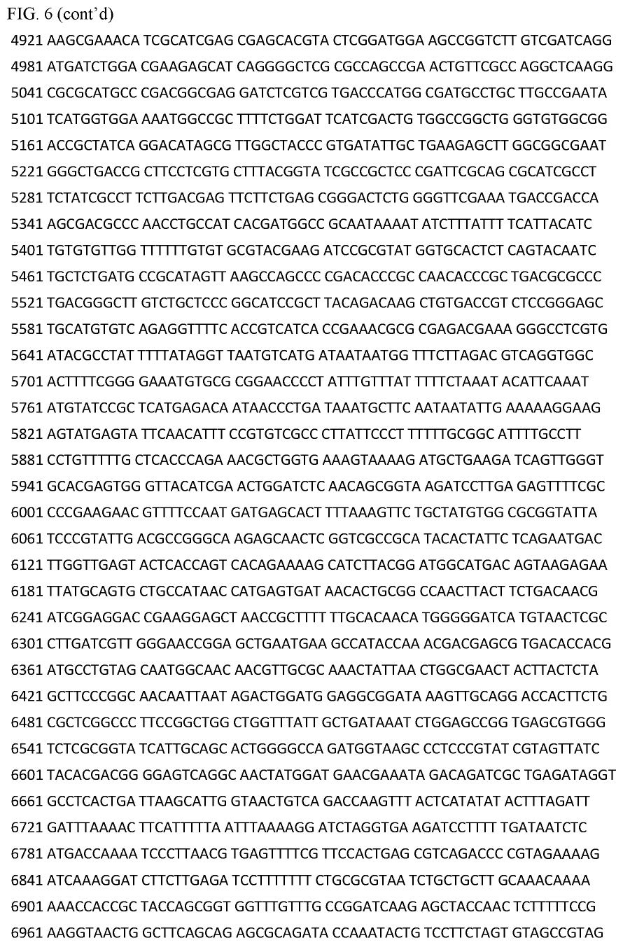

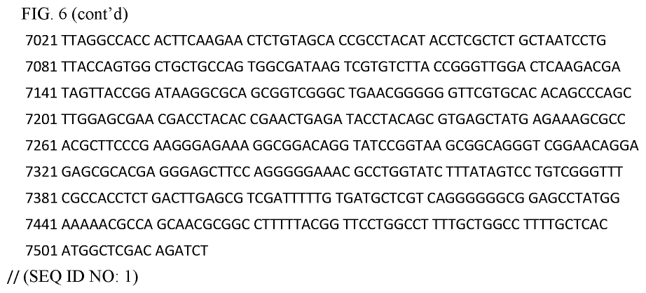

Quantification of Recombination: Genomic DNA from 3 million cells was isolated using a Qiagen DNEasy kit. AttR is the result of recombination between attP and attB. Quantitative Polymerase Chain Reaction (QPCR) using sybr-green dye was performed to quantify attR in the cells using a forward primer in the attP sequence in the dock and a reverse primer in the attB sequence in the transgene. Amplification using this primer pair will only detect the transgene plasmid when it is recombined into the dock and not free, randomly integrated, or pseudo-attP integrated transgene plasmid. Similarly, this primer pair will not detect unrecombined (empty) dock sequence. The number of PCR cycles needed to cross a fluorescence intensity threshold (Ct value) was determined for this primer set as well as a primer set for an internal CHO reference gene. Primers specific to the EPR portion of the Dock (FIGS. 5 and 6) were used to rank clones based on EPR GCI. Gene Copy Index values were calculated by subtracting the Ct value of the reference gene from the Ct value of the attR primer set. Note that GCI values are a logarithmic, not linear, in nature such that a change of 1 unit at the low end of the scale, ex from GCI=1 to GCI=3, represents a difference of only a few copies whereas a change in one unit at the higher end of the scale, ex from GCI=6 to GCI=7 can represent a difference of numerous copies. In some cases, a plasmid containing the desired amplicons and of known concentration was also subjected to QPCR and this data was subjected to linear regression analysis to more precisely determine the number of copies present.

Fed-Batch Production: For the fed batch production, 50 ml spin tubes were seeded at 600,000 cells per ml in 20 mls of Ex-Cell Advanced CHO Fed-Batch™ media (MilliporeSigma) and incubated in a humidified (70-80%) shaking incubator at 250 rpm with 5% CO₂ and temperature of 37° C. (34° C. starting day 4). Cultures were fed every other day starting on day 2 with 6.25% (V:V) of a feed blend containing 66% Ex-cell Advanced CHO Feed 1™ and 33% Cellvento 4Feed (MilliporeSigma). Glucose was monitored daily and supplemented if the level dropped below 5 g/L. Cultures were terminated when viabilities were ≤70% or at the end of day 20.

### Results

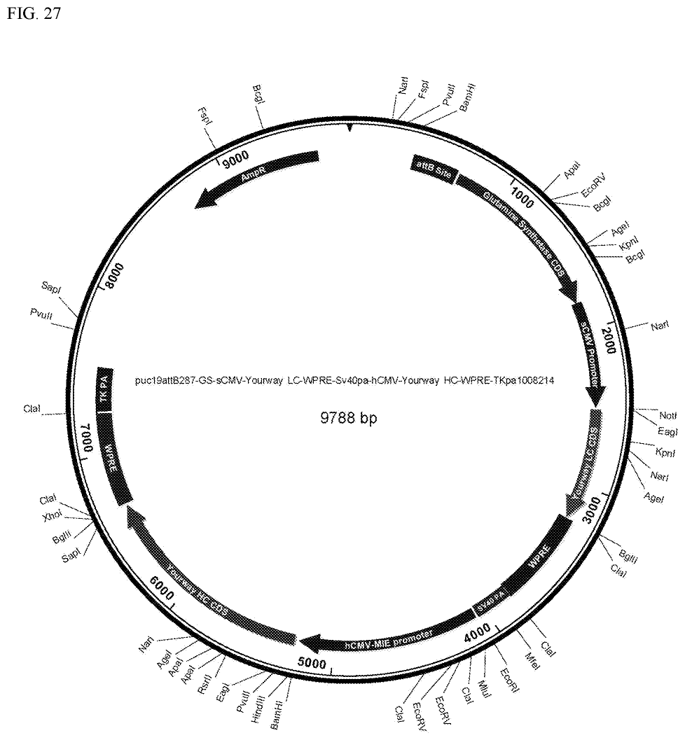

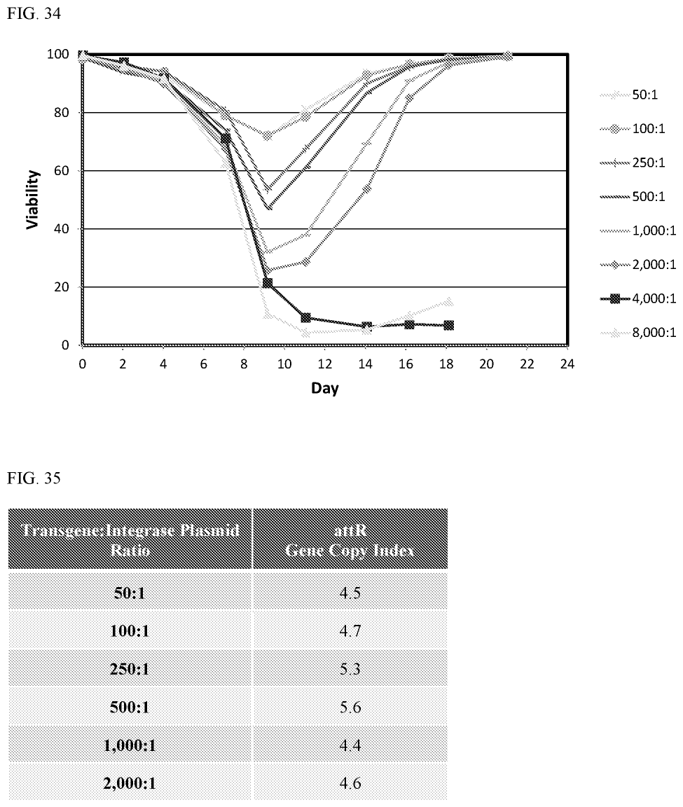

To isolate a high Dock copy number clone, the Dock cell pool made with 9 rounds of transduction was subjected to single cell cloning using the Berkely Lights, Beacon® instrument. Clones were isolated, expanded, and subjected to QPCR using primers specific to the EPR region of the Dock. Clone 1F7 contained approximately 181 copies of the Dock plasmid per cell and was selected for further experimentation. Dock clone 1F7 was co-transfected with Transgene-Yourway-LWHW plasmid expressing both the light and heavy antibody chains, and Integrase plasmid, FIGS. 27+28 and 5+6 with ratios ranging from 50:1 to 8,000:1. The resulting pools were subjected to selection through Glutamine withdrawal, FIG. 34. Pools with ratios 4,000:1 and 8,000:1 did not survive selection. QPCR analysis of attR on surviving pools, FIG. 35, indicates that larger plasmids up to at least 9.8 kilobases can be efficiently integrated with the technology and the optimal Transgene:Integrase plasmid ratio plasmids of this size is 500:1.

### Example 5

After observing relatively high integration efficiency in the 1F7 Dock clone, we next sought to determine if clones derived from pools with high levels of integrated transgenes could contain even higher levels of transgene integration and to determine production capacity of these clones.

Transfection and Selection of Dock Pooled Cell Line: 3 million cells were incubated with a precomplexed mixture containing 2 ug (total) of plasmid Transgene and Integrase DNA (FIGS. 13, 14, 5 and 6, respectively) and 8 microliters of ExpiFectamine CHO™ (ThermoFisher Scientific) in a final volume of 500 microliters. Pooled cell lines were allowed to recover in the presence of media supplemented with 6 mM glutamine until viability returned to greater than 95%. Cells were then transferred to media lacking glutamine. Viability was monitored and media was replaced weekly until the resulting selected cell pools returned to greater than 95% viability.

Cloning of Pools with Integrated Transgenes: Pools with integrated Transgenes were cloned using the Berkeley Lights, Beacon instrument. The Spotlight® assay was used to measure relative productivity of this clones. Clones with the highest productivity were exported from the machine and expanded.

Quantification of Recombination: Genomic DNA from 3 million cells was isolated using a Qiagen DNEasy kit. AttR is the result of recombination between attP and attB. Quantitative Polymerase Chain Reaction (QPCR) using sybr-green dye was performed to quantify attR in the cells using a forward primer in the attP sequence in the dock and a reverse primer in the attB sequence in the transgene. Amplification using this primer pair will only detect the transgene plasmid when it is recombined into the dock and not free, randomly integrated, or pseudo-attP integrated transgene plasmid. Similarly, this primer pair will not detect unrecombined (empty) dock sequence. The number of PCR cycles needed to cross a fluorescence intensity threshold (Ct value) was determined for this primer set as well as a primer set for an internal CHO reference gene. Primers specific to the attP, which is present only in unintegrated Docks were used to estimate the portion of filled docs. Gene Copy Index values were calculated by subtracting the Ct value of the reference gene from the Ct value of the attR primer set. Note that GCI values are a logarithmic, not linear, in nature such that a change of 1 unit at the low end of the scale, ex from GCI=1 to GCI=3, represents a difference of only a few copies whereas a change in one unit at the higher end of the scale, ex from GCI=6 to GCI=7 can represent a difference of numerous copies. In some cases, a plasmid containing the desired amplicons and of known concentration was also subjected to QPCR and this data was subjected to linear regression analysis to more precisely determine the number of copies present.

### Results

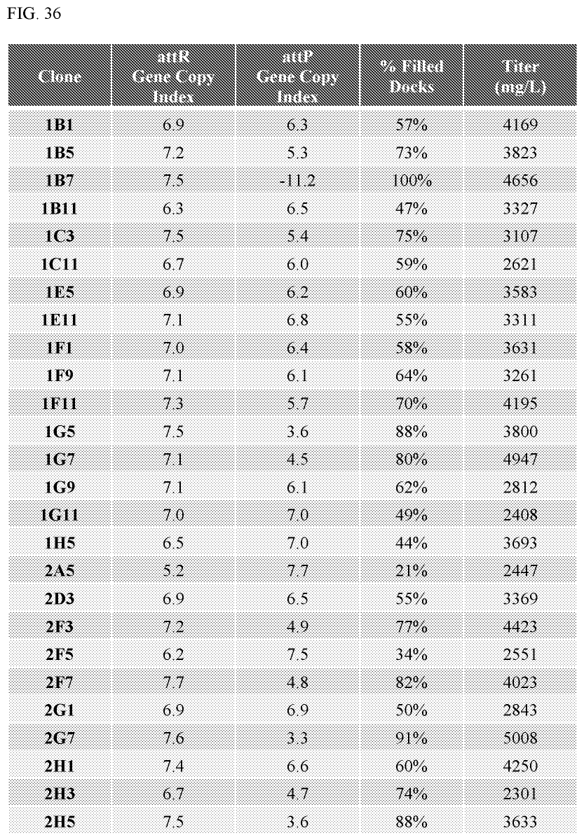

Dock clone 1F7 was co-transfected with Transgene-Anyway and Integrase plasmids (FIGS. 13, 14, 5 and 6, respectively) and the resulting pools were subjected to selection through Glutamine withdrawal. attR GCI for the selected pool was 6.9. This pool was subjected to single cell cloning using the Berkely Lights, Beacon instrument. Clones were ranked and exported based on relative Anyway expression using the Spotlight® Assay. 27 clones were expanded and AttR GCIs in these clones, FIG. 36, ranged from 5.2 to 7.5. AttP GCI, which measures empty Dock, was also measured for these clones, allowing us to estimate the portion of filled Docks in each clone, FIG. 36. The average percent fill in these clones was 65%. This represents roughly 118 copies of integrated Transgene plasmid. Clone 1B7 had an attR GCI of 7.5 which was equivalent to the attP (empty Dock) GCI for the parental Dock clone 1F7. Surprisingly, we were not able to detect attP in this clone using two different primer pairs. These data indicate that, surprisingly, after only a single transfection we were able to obtain a clone with all approximately 181 dock sites filled with transgene.

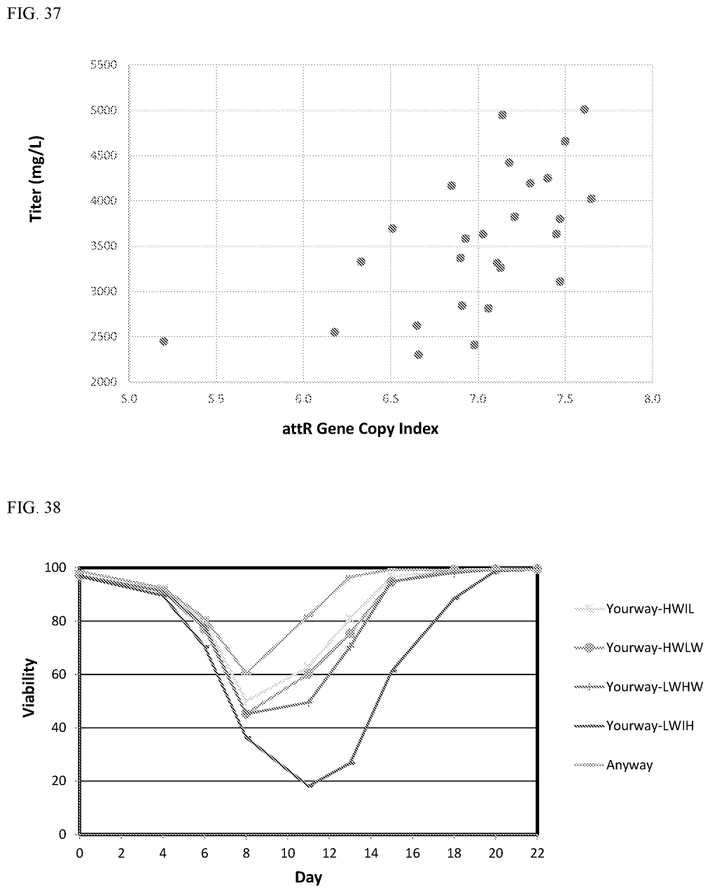

To determine their protein production capacity, generic fed-batch productivity analysis was performed on all these clones with final titers also shown in FIG. 36. High attR GCI levels was associated with high final titer, FIG. 37, indicating that, as expected, increasing amounts of targeted integration of transgenes into highly active dock sites results in increased protein production capacity of the cell line. These data also suggest that we have not yet saturated the production capacity of these cells even with approximately 181 copies integrated.

### Example 6

After observing relatively high integration efficiency and expression of a fusion protein in the 1F7 Dock clone, we next sought to determine if we could also use this system to integrate and express monoclonal antibodies with both heavy and light chains on the same Transgene plasmid.

Transfection and Selection of Dock Pooled Cell Line: 3 million cells were incubated with a precomplexed mixture containing 2 ug (total) of plasmid Transgene and Integrase DNA, and 8 microliters of ExpiFectamine CHO™ (ThermoFisher Scientific) in a final volume of 500 microliters. Pooled cell lines were allowed to recover in the presence of media supplemented with 6 mM glutamine until viability returned to greater than 95%. Cells were then transferred to media lacking glutamine. Viability was monitored and media was replaced weekly until the resulting selected cell pools returned to greater than 95% viability.

Quantification of Recombination: Genomic DNA from 3 million cells was isolated using a Qiagen DNEasy kit. AttR and attL are the result of recombination between attP and attB. Quantitative Polymerase Chain Reaction (QPCR) using sybr-green dye was performed to quantify attR in the cells using a forward primer in the attP sequence in the dock and a reverse primer in the attB sequence in the transgene. Amplification using this primer pair will only detect the transgene plasmid when it is recombined into the dock and not free, randomly integrated, or pseudo-attP integrated transgene plasmid. Similarly, this primer pair will not detect unrecombined (empty) dock sequence. The number of PCR cycles needed to cross a fluorescence intensity threshold (Ct value) was determined for this primer set as well as a primer set for an internal CHO reference gene. Primers specific to the attP, which is present only in unintegrated Docks were used to estimate the portion of filled docs. Gene Copy Index values were calculated by subtracting the Ct value of the reference gene from the Ct value of the attR primer set. Note that GCI values are a logarithmic, not linear, in nature such that a change of 1 unit at the low end of the scale, ex from GCI=1 to GCI=3, represents a difference of only a few copies whereas a change in one unit at the higher end of the scale, ex from GCI=6 to GCI=7 can represent a difference of numerous copies. In some cases, a plasmid containing the desired amplicons and of known concentration was also subjected to QPCR and this data was subjected to linear regression analysis to more precisely determine the number of copies present.

Fed-Batch Production: 50 ml spin tubes were seeded at 600,000 cells per ml in 20 mls of Ex-Cell Advanced CHO Fed-Batch™ media (MilliporeSigma) and incubated in a humidified (70-80%) shaking incubator at 250 rpm with 5% CO₂ and temperature of 37° C. (34° C. starting day 4). Cultures were fed every other day starting on day 2 with 6.25% (V:V) of a feed blend containing 66% Ex-Cell Advanced CHO Feed 1TM and 33% Cellvento 4Feed (MilliporeSigma). Glucose was monitored daily and supplemented if the level dropped below 5 g/L. Cultures were terminated when viabilities were ≤70% or at the end of day 20.

Protein Gel Electrophoresis. Supernatants from Fed Batch production (see above) were harvested and clarified. 3 ug of each antibody or Fc fusion protein was mixed with LDS loading buffer, with or without the addition of a denaturing agent. Denatured samples were also heated to 70 degrees for 10 minutes prior to electrophoresis. All samples were loaded onto a NuPAGE Novex 4-12% Bis-Tris gel (Invitrogen), and electrophoresed in 1X MES buffer for 15 minutes at 60V, and then 105 minutes at 100V. The gel was then rinsed with deionized water, and stained with SYPRO-Ruby. Stained gels were imaged, and the “negative” image (color-reversed) of the stained gel is found in FIG. 40.

### Results

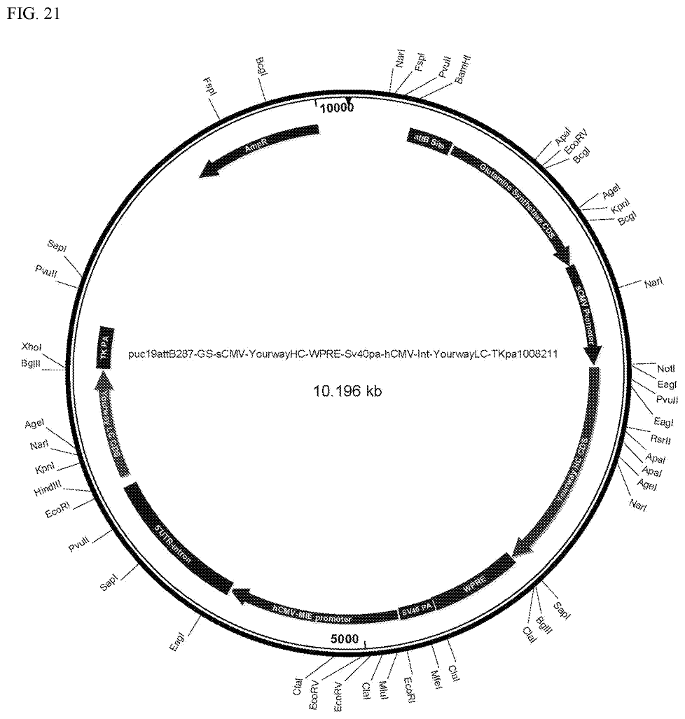

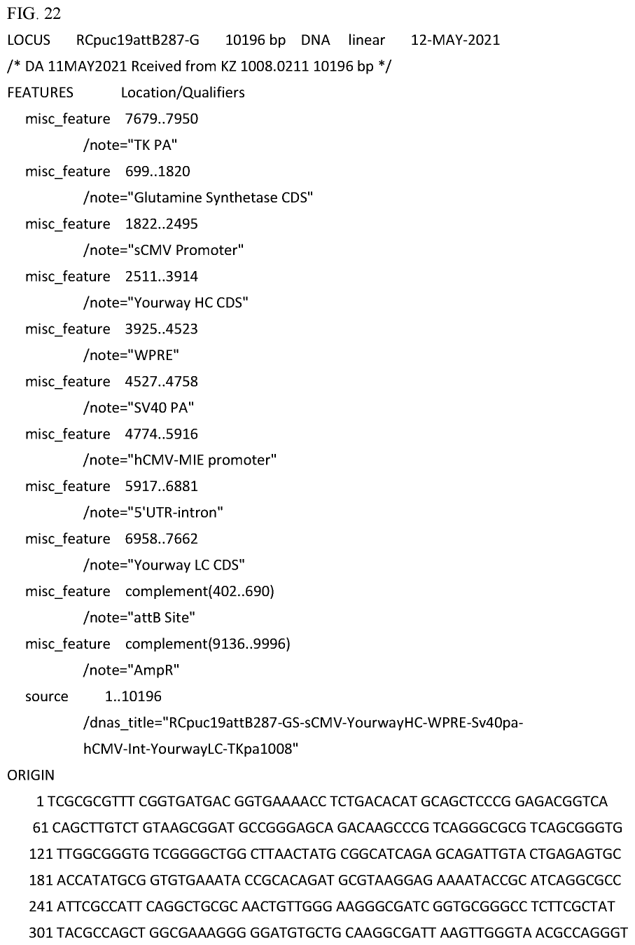

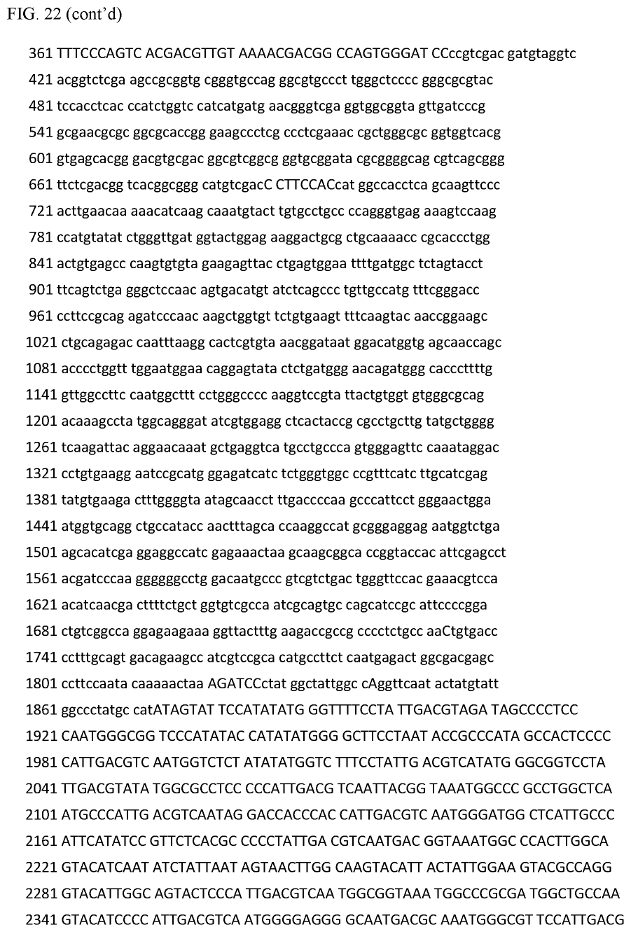

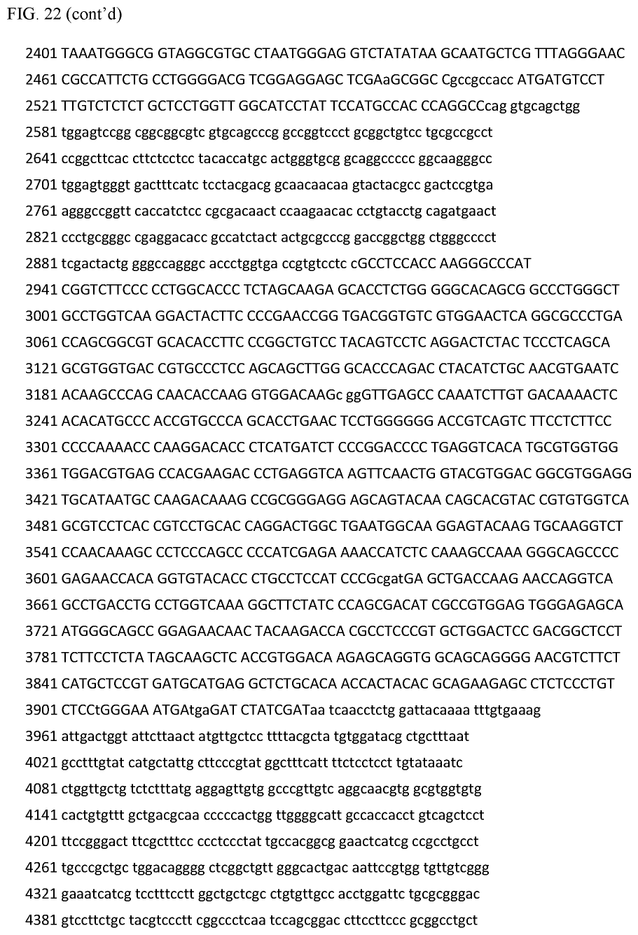

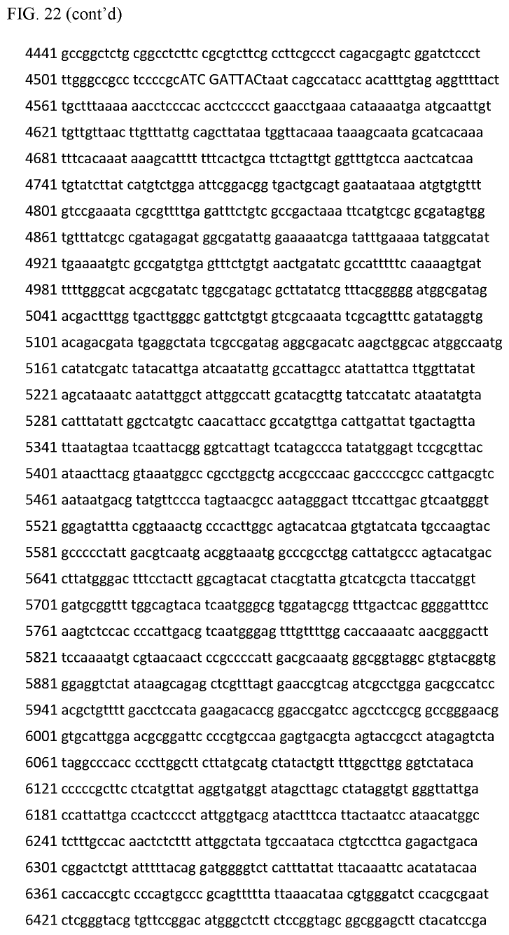

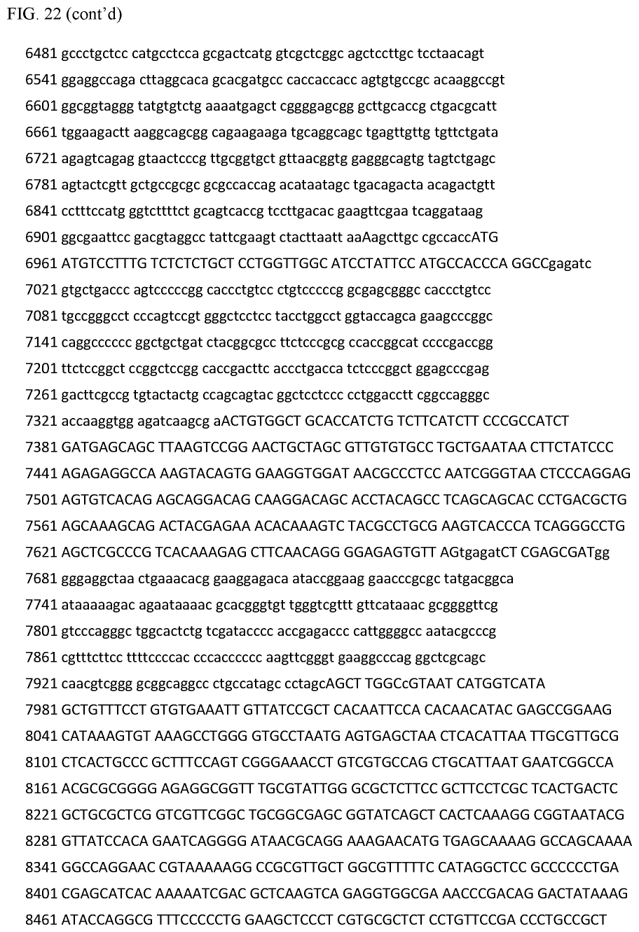

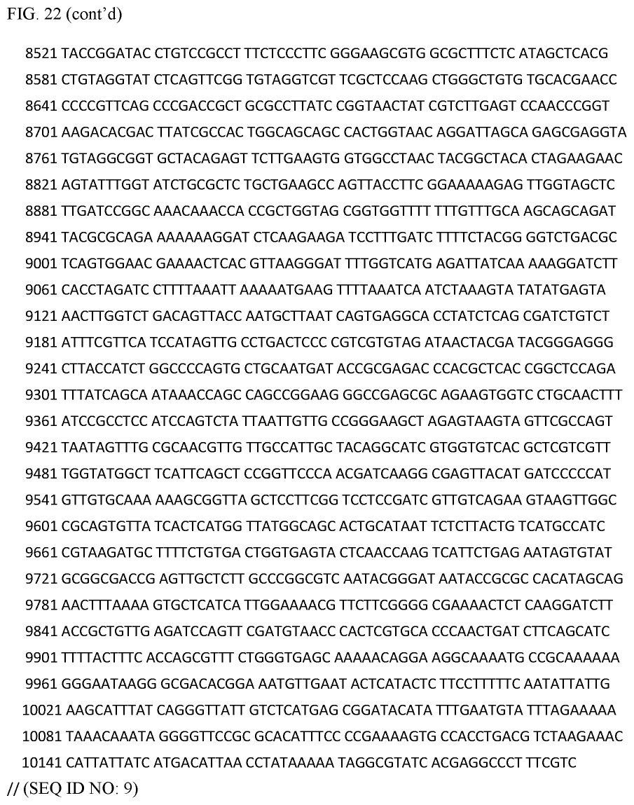

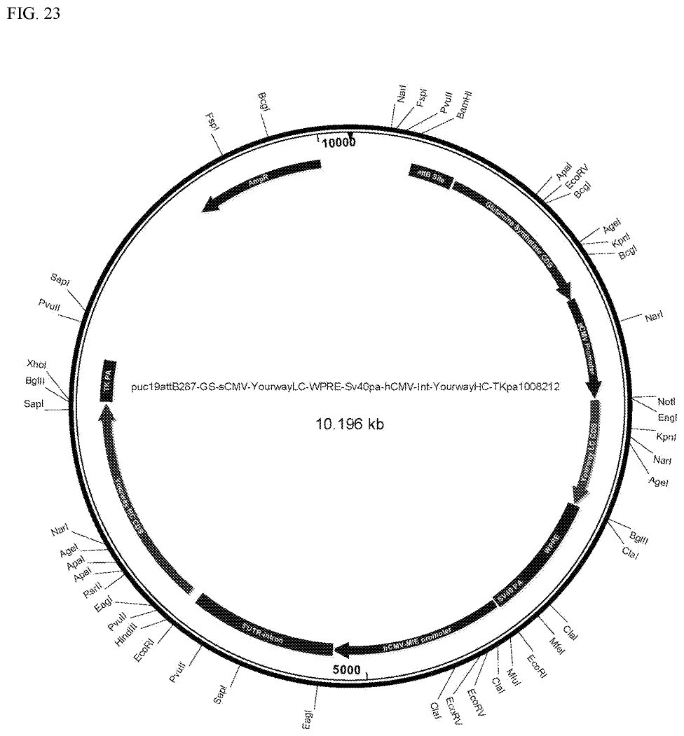

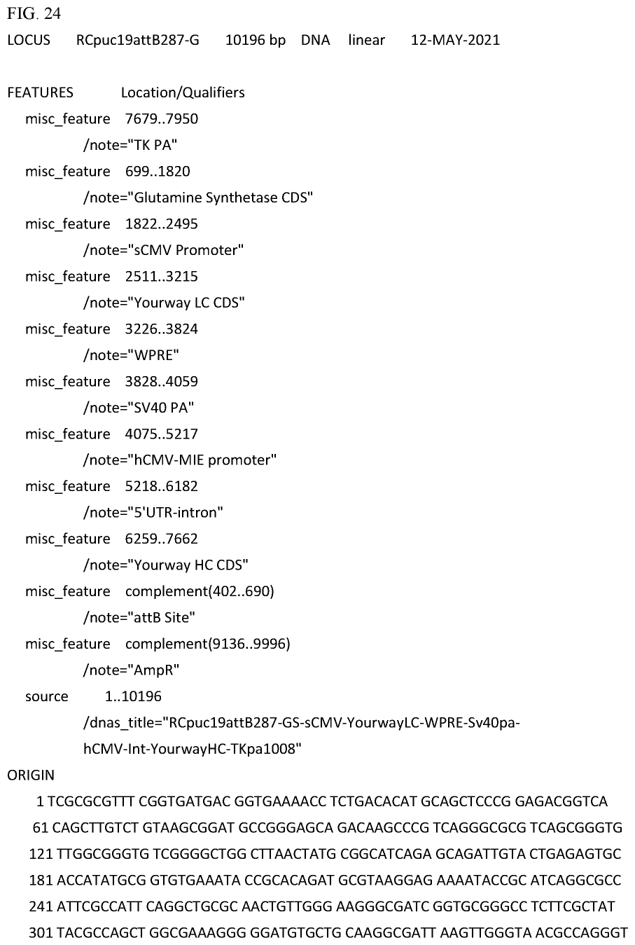

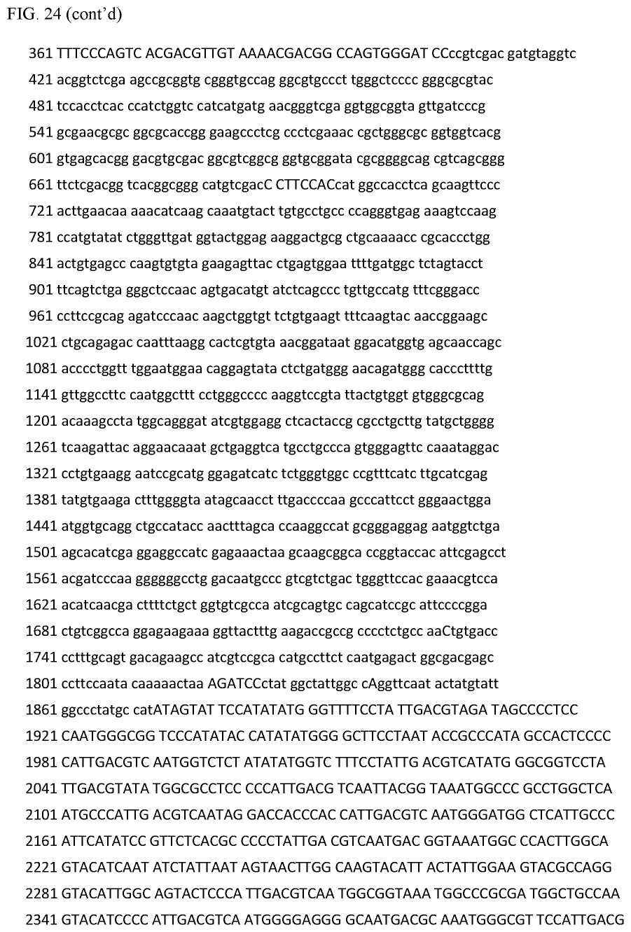

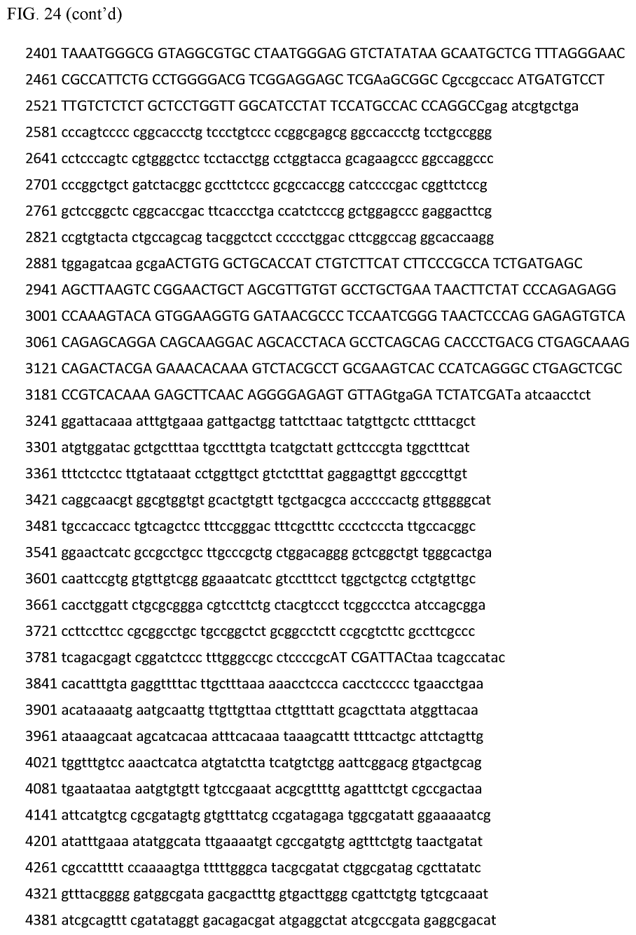

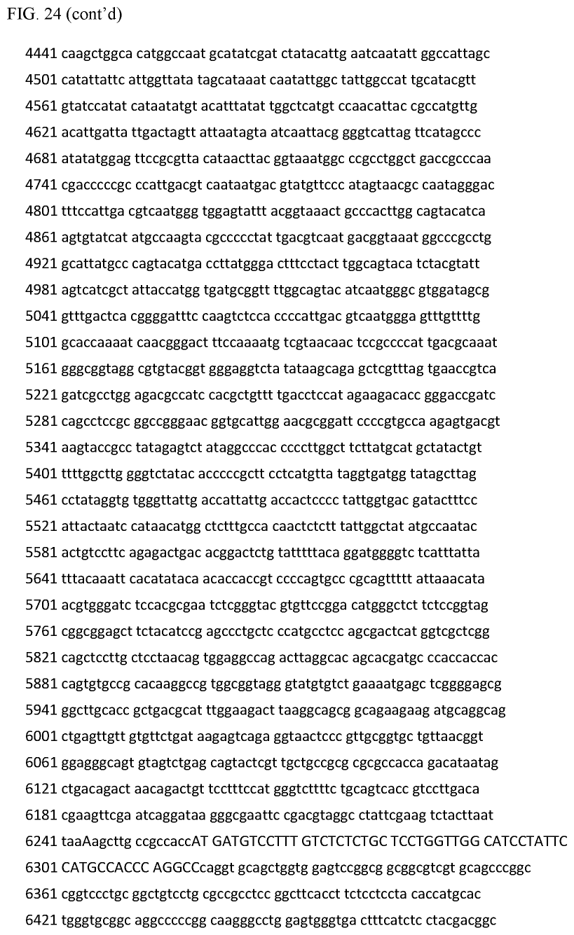

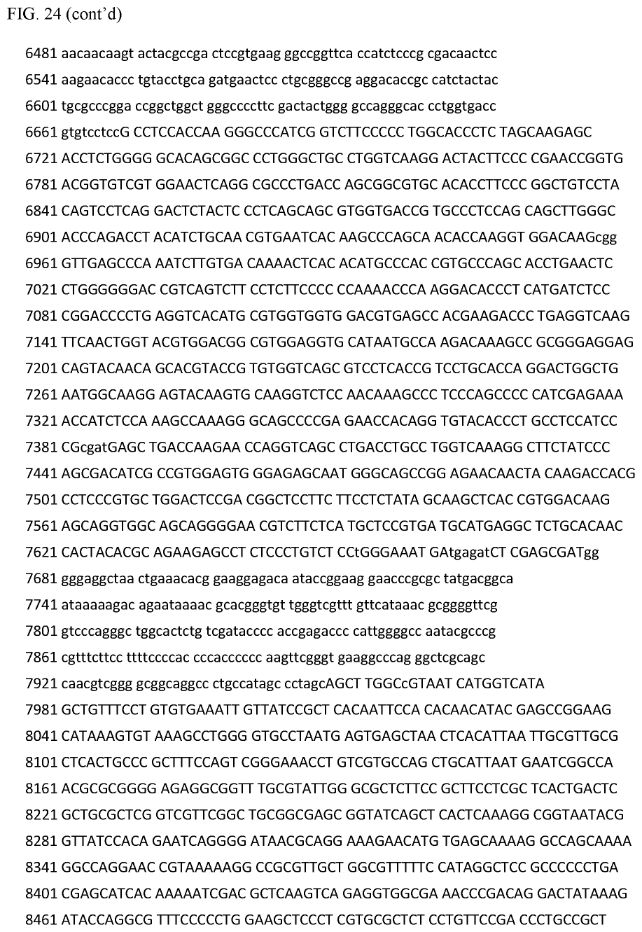

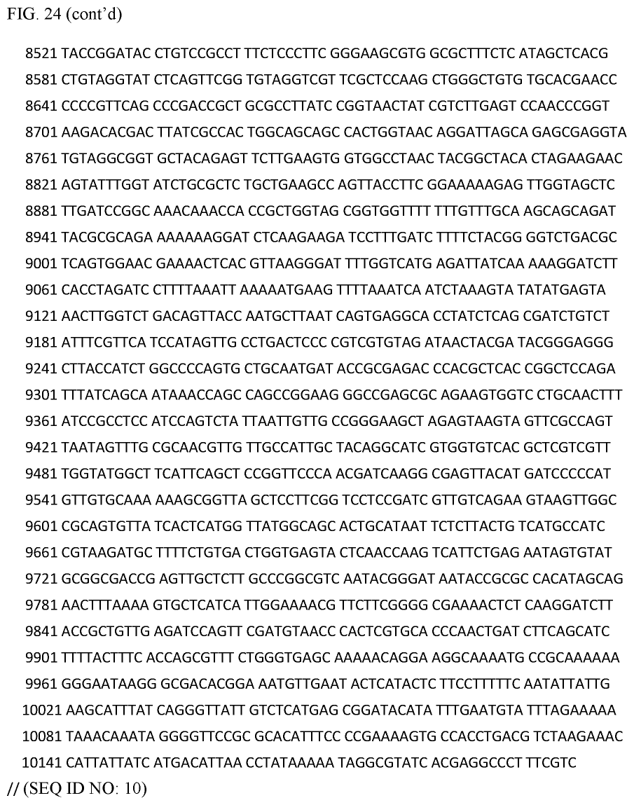

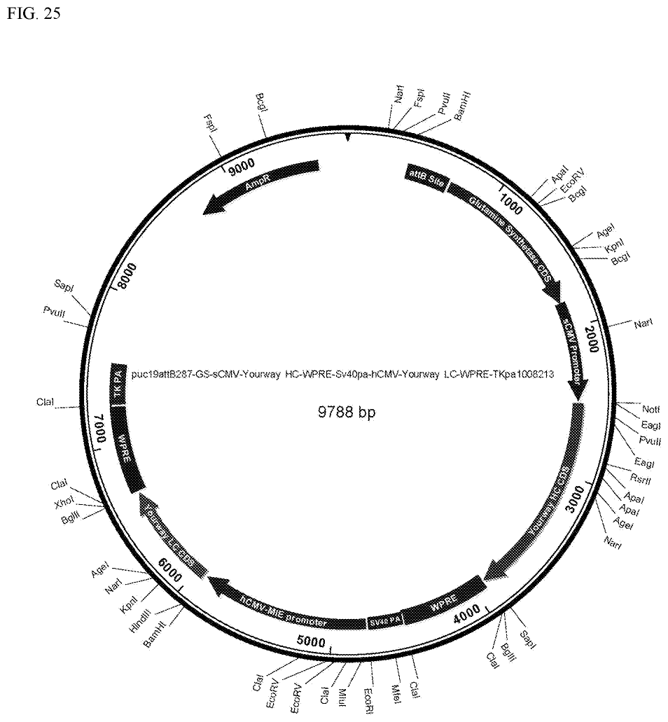

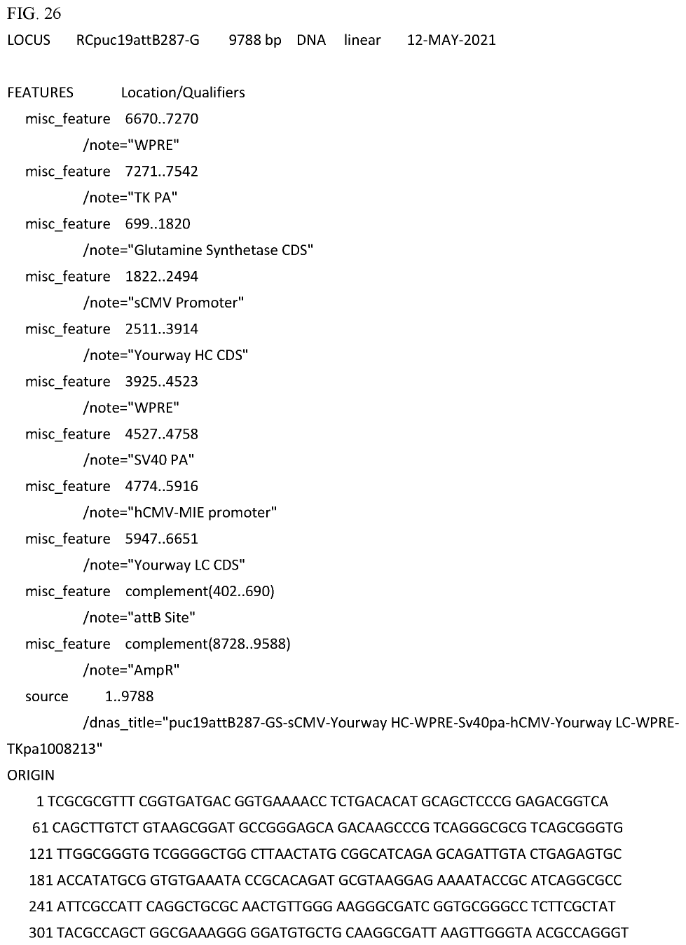

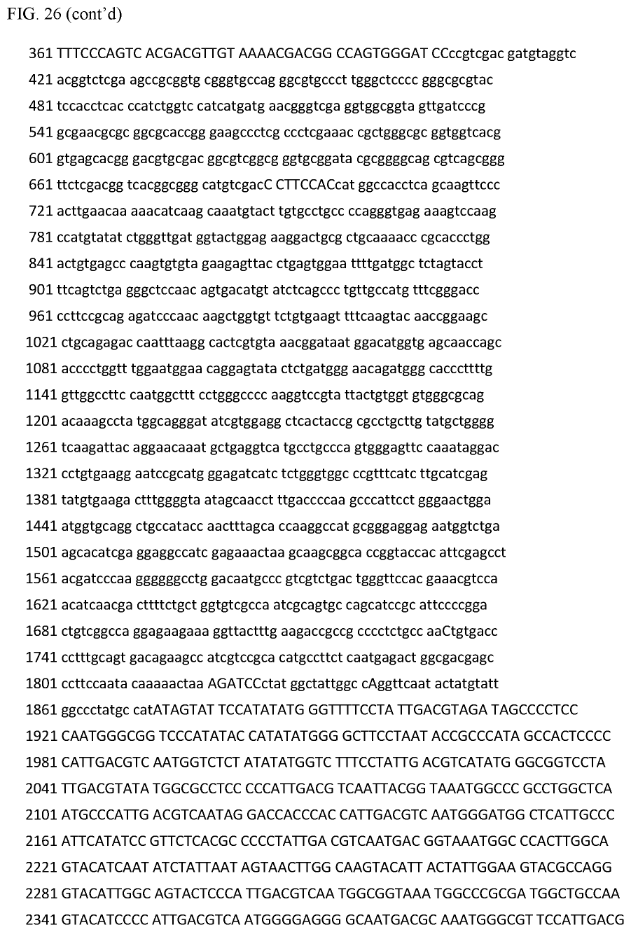

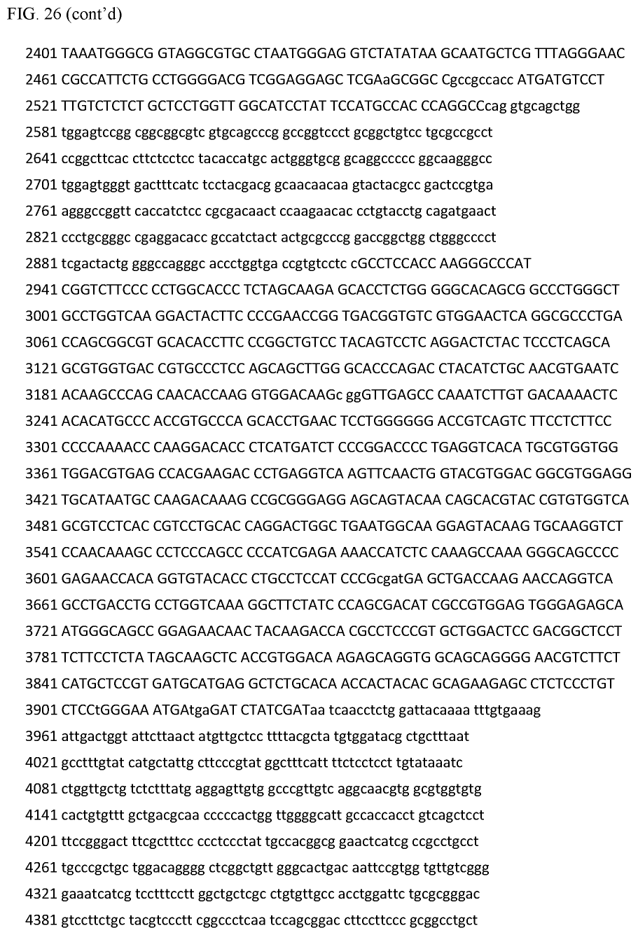

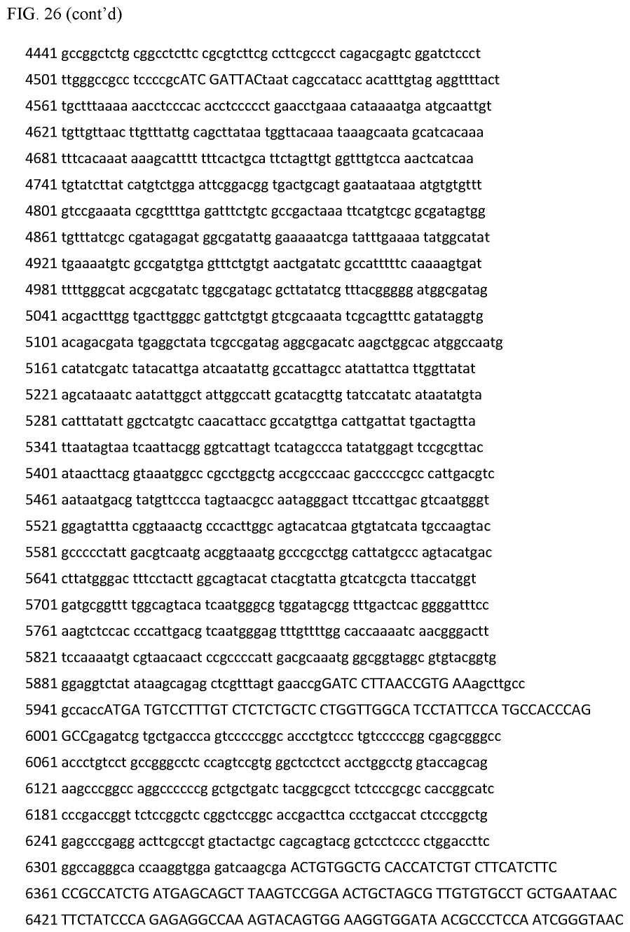

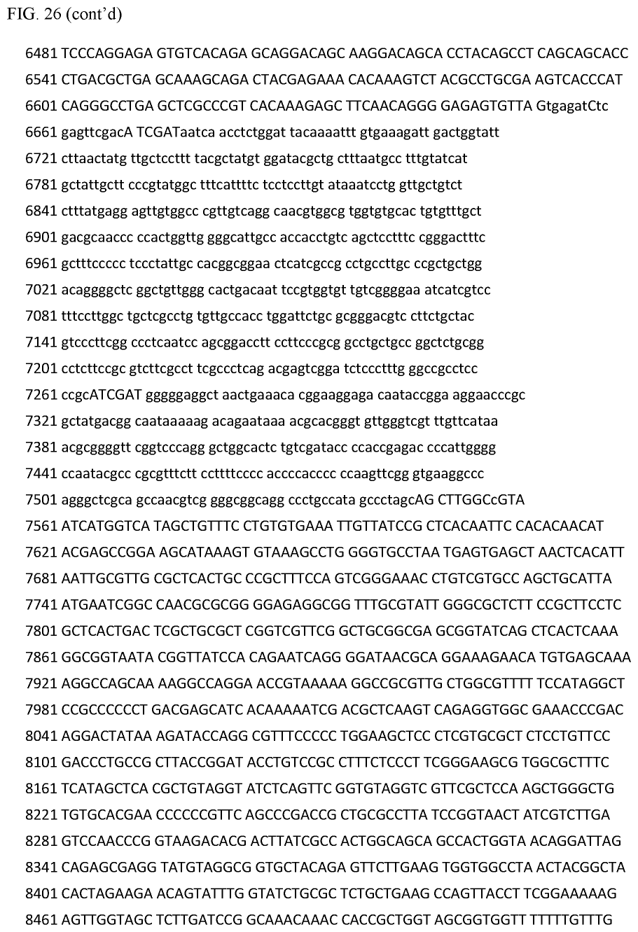

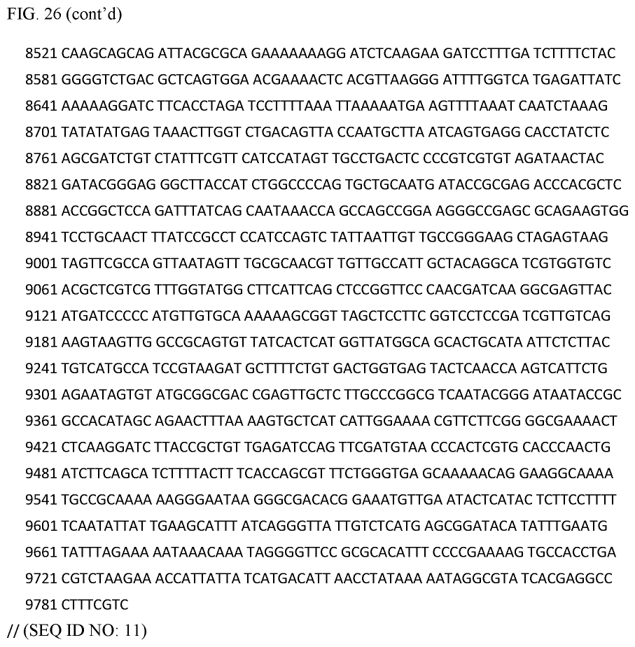

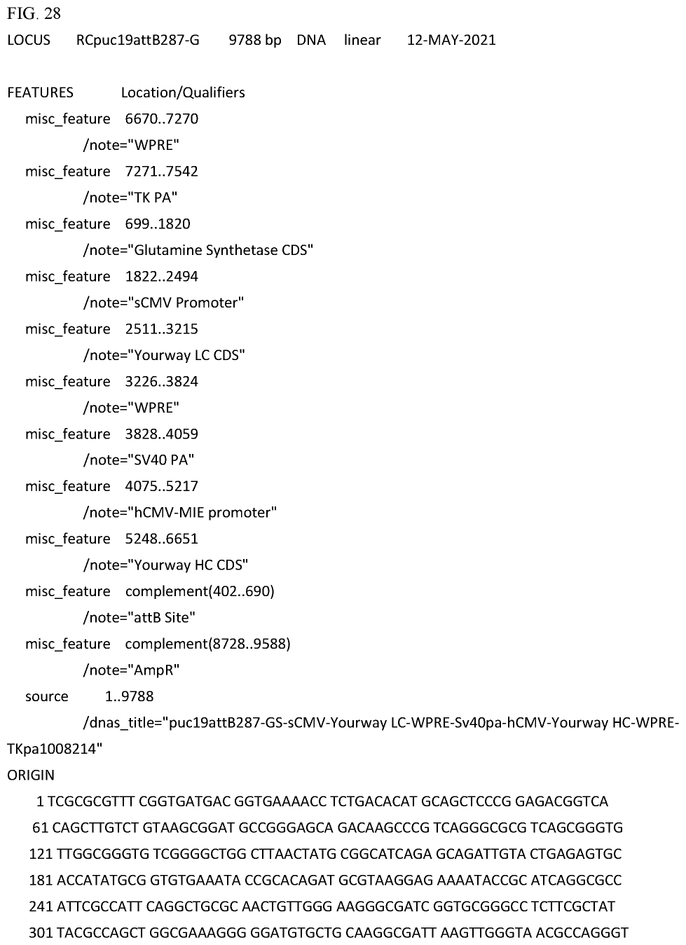

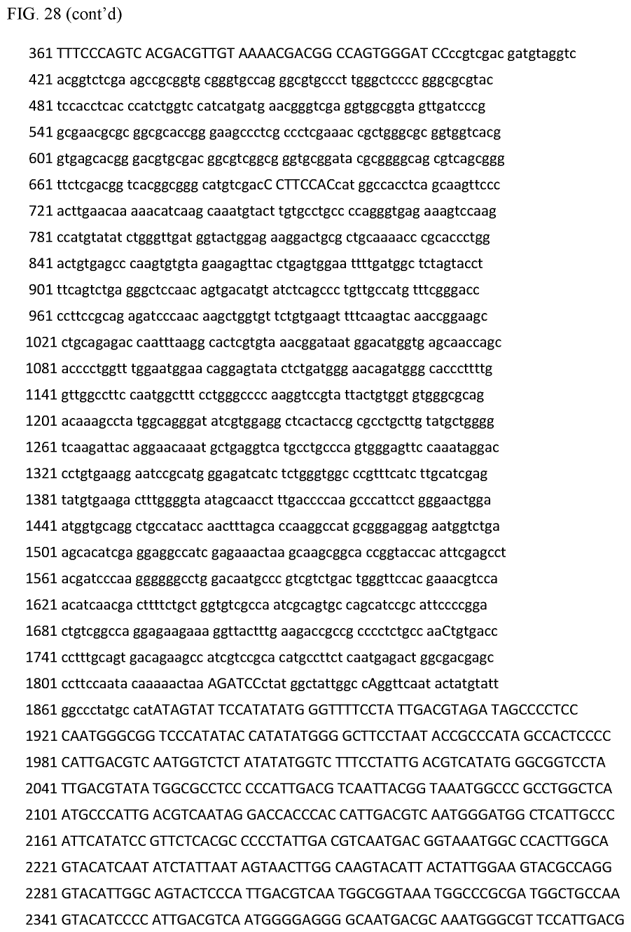

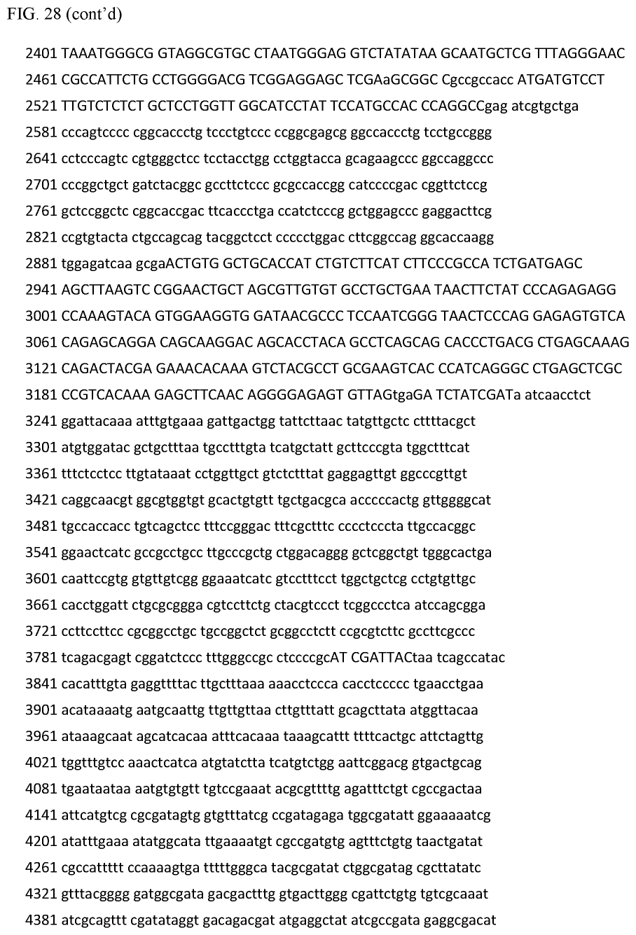

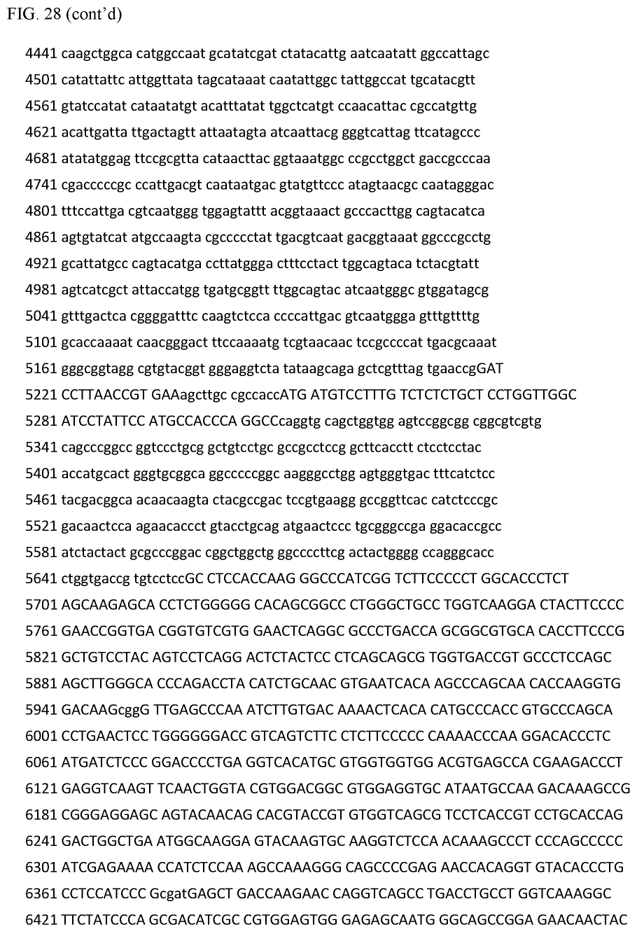

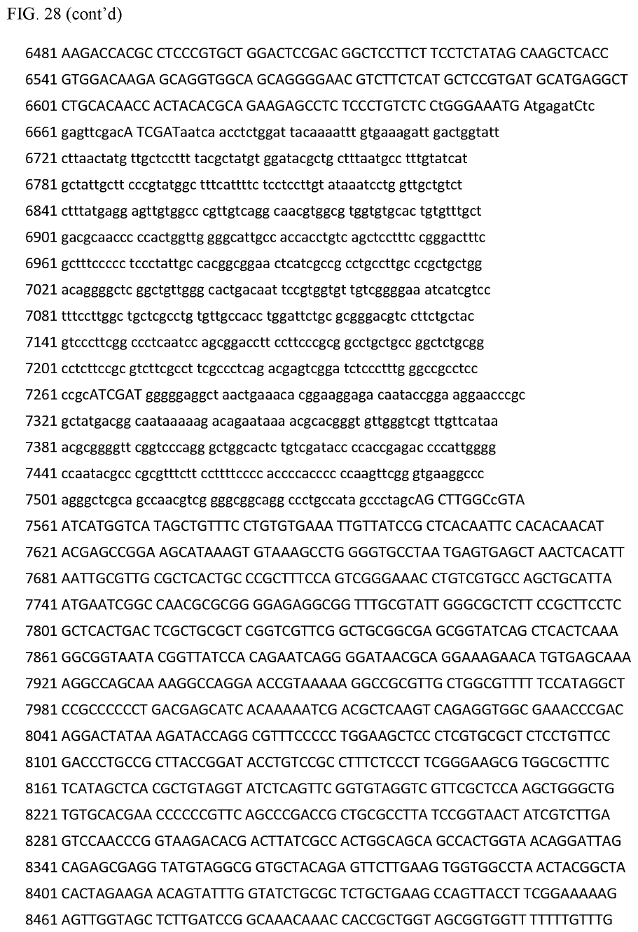

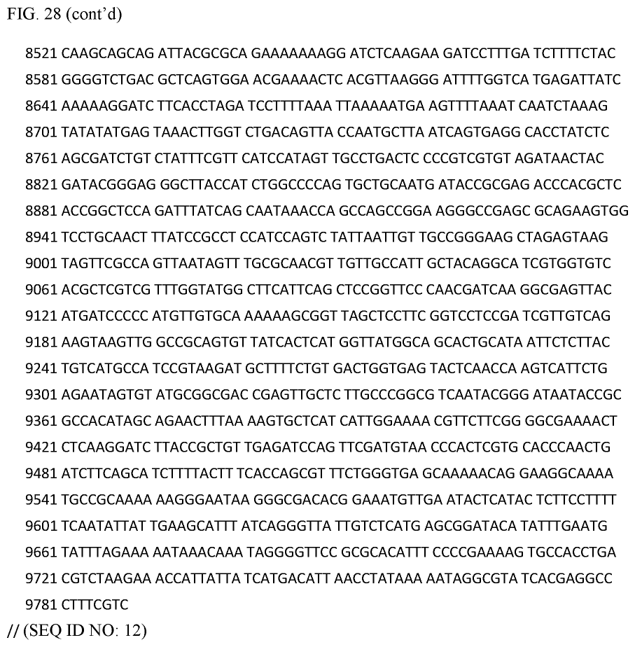

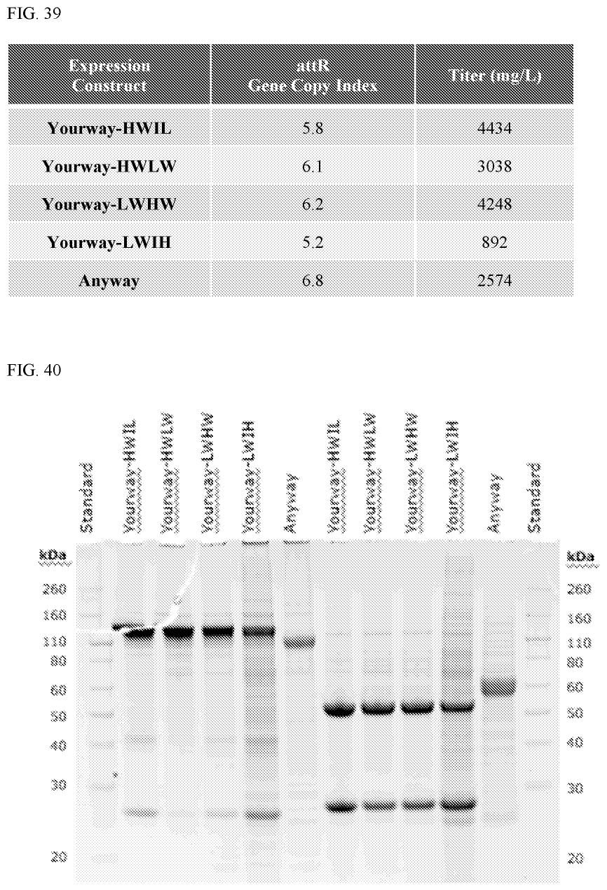

Both expression and purification of monoclonal antibodies is well known to be sensitive to the relatives amounts of light chain and heavy chain expressed. Our system is designed to integrate the light chain and heavy chain in a 1:1 gene ratio. To optimize the relative expression of each chain, we designed and tested four different expression constructs that contain different gene orders and enhancer elements (See FIGS. 21, 22, 23, 24, 25, 26, 27 and 28). All constructs tested do not contain a GS promoter and contain strong promoters and poly A sequences for both heavy chain and light chain genes. In the first construct, referred to as HWIL (to highlight differences between constructs), the heavy chain coding sequence (H) is expressed from the upstream promoter and is followed by the Woodchuck Post-transcriptional Regulatory Element (W or WPRE). The light chain coding sequence (L) is expressed from the downstream promoter and is preceded by an intron sequence (I). The remaining three expression constructs follow this same nomenclature. Dock clone 1F7 containing approximately 181 copies of Dock, was co-transfected with all four Transgene-Yourway plasmids or Transgene-Anyway plasmid (individually) and Integrase plasmids (FIGS. 5+6, 21+22, 23+24, 25+26, 27+28, 13+14) and the resulting pools were subjected to selection through Glutamine withdrawal, FIG. 38. Interestingly, pools transfected with the LWIH plasmid recovered more slowly from selection than other plasmids. QPCR analysis of the resulting pools, FIG. 38, showed that a high level of transgene integration was attained-similar to previous examples despite the larger size of these plasmids. Fed-batch productivity was also performed to determine the protein production capacity of these pools, FIG. 39. Three of the four expression plasmids showed robust expression with HWIL and LWHW providing the highest titers. The resulting proteins were subjected to both non-reduced and reduced SDS-PAGE analysis, FIG. 40, to assess the relative expression of the heavy and light chains and the assembly of the mature antibody. All four expression plasmids showed a high portion of mature antibody formation at 150 kDa relative to free light and heavy chains. All four antibody expression plasmids also had a slight excess of light chain expression which is desirable for protein A purification to minimize purification of free heavy chain. Similarly, expression of a single chain fusion protein, Anyway, showed both high titer, FIG. 39, and high portion of mature, dimerized protein of the predicted size.

### Example 7

Next we wanted to determine the production stability of pools generated using this technology as this is a necessary attribute for manufacturing.

Retrovector Production and Transduction to Create the Dock Parental Cell Line: The Dock construct, FIGS. 7 and 8, was introduced into a HEK 293 cell line that constitutively produces the MLV gag, pro, and pol proteins. An envelope containing expression plasmid was also co-transfected with the each of the gene constructs. The co-transfection resulted in the production of replication incompetent high titer retrovector that was concentrated by ultracentrifugation and used for cell transductions of the CHOZN Chinese Hamster Ovary parental cell line (1,2). 5 sequential rounds of transduction were performed, and cells were routinely maintained media supplemented with 6 mM glutamine.

Transfection and Selection of Dock Pooled Cell Line: 3 million cells were incubated with a precomplexed mixture containing 2 ug (total) of plasmid Transgene and Integrase DNA, and 8 microliters of ExpiFectamine CHO™ (ThermoFisher Scientific) in a final volume of 500 microliters. Pooled cell lines were allowed to recover in the presence of media supplemented with 6 mM glutamine until viability returned to greater than 95%. Cells were then transferred to media lacking glutamine. Viability was monitored and media was replaced weekly until the resulting selected cell pools returned to greater than 95% viability.

Fed-Batch Production-Ex-cell: 50 ml spin tubes were seeded at 600,000 cells per ml in 20 mls of Ex-Cell Advanced CHO Fed-Batch™ media (MilliporeSigma) and incubated in a humidified (70-80%) shaking incubator at 250 rpm with 5% CO₂ and temperature of 37° C. (34° C. starting day 4). Cultures were fed every other day starting on day 2 with 6.25% (V:V) of a feed blend containing 66% Ex-Cell Advanced CHO Feed 1TM and 33% Cellvento 4Feed (MilliporeSigma). Glucose was monitored daily and supplemented if the level dropped below 5 g/L. Cultures were terminated when viabilities were ≤70% or at the end of day 20.

Fed-Batch Production-ActiPro: 50 ml spin tubes were seeded at 600,000 cells per ml in 20 mls of Hyclone ActiPro™ media (Activa Life Sciences) and incubated in a humidified (70-80%) shaking incubator at 250 rpm with 5% CO₂ and temperature of 37° C. (34° C. starting day 4). Cultures were fed every other day starting on day 2 with 3% (V:V) Hyclone Cell Boost 7A and 0.3% Hyclone Cell Boost 7b (Activa Life Sciences). Glucose was monitored daily and supplemented if the level dropped below 5 g/L. Cultures were terminated when viabilities were ≤70% or at the end of day 20.

### Results

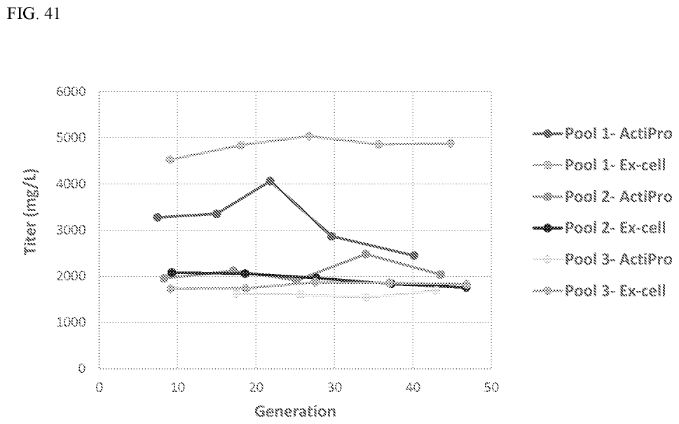

To determine the production stability of pools expressing the Anyway fusion protein, the Dock cell pool made with 9 rounds of transduction was co-transfected with Transgene-Anyway and Integrase plasmids, FIGS. 13+14 and 5+6. Resulting pools were selected by Glutamine withdrawal. Three pools were continually passaged and aliquots were frozen weekly for more than 40 generations. Once 40 generations were reached for all pools, vials from previously frozen generations were thawed and fed-batch productivity was performed using two different media/feed strategies. Final titers from the fed-batch productivities, FIG. 41, shows that even after continual culture for over 40 generations, protein titers remained stable in all three pools indicating both robust genetic stability of integrated transgene plasmids as well as stable expression from the integrated transgene plasmids both critical attributes for the use of this technology in drug substance manufacturing.

All publications and patents mentioned in the above specification are herein incorporated by reference. Various modifications and variations of the described method and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention that are obvious to those skilled in the field of this invention are intended to be within the scope of the following claims.

1. A nucleic acid construct for expression of a protein of interest comprising the following elements in operable association in 5′ to 3′ order:
optionally, a first promoter sequence;
a selectable marker sequence;
a second promoter sequence;
a nucleic acid sequence encoding a first protein of interest that is operably linked to the second promoter sequence; and
a poly A signal sequence;
the nucleic acid construct further comprising at least one insertion element at a position or positions selected from the group consisting of 5′ to the optional first promoter or selectable marker sequence, 3′ to the poly A signal sequence, between the optional first promoter and the poly A signal sequence, between the selectable marker and the second promoter sequence, and both 5′ to the optional first promoter sequence or the selectable marker sequence and 3′ to the poly A signal sequence.

2. The nucleic acid construct of claim 1, wherein the nucleic acid construct does not comprise a poly A signal sequence between the selectable marker and the second promoter.

3. The nucleic acid construct of claim 1, wherein the selectable marker is adjacent to the second promoter.

4. The nucleic acid construct of claim 1, wherein the second promoter is adjacent to the nucleic acid sequence encoding the first protein of interest.

5. The nucleic acid construct of claim 1, wherein the nucleic acid construct comprises an extending packaging region between the first promoter and the selectable marker.

6. The nucleic acid construct of claim 5, wherein the EPR comprises multiple potential Kozak sequences and/or ATG translation start sites.

7. The nucleic acid construct of claim 1, wherein the first promoter sequence is a weak promoter sequence.

8. The nucleic acid construct of claim 1, wherein the first promoter sequence is selected from the group consisting of SIN-LTR, SV40, E. coli lac, E. coli trp, phage lambda PL, phage lambda PR, T3, T7, cytomegalovirus (CMV) immediate early, herpes simplex virus (HSV) thymidine kinase, alpha-lactalbumin, human elongation factor 1 alpha (hEF1alpha), and mouse metallothionein-I promoter sequences.

9. (canceled)

10. The nucleic acid construct of claim 1, wherein the selectable marker sequence is an amplifiable selectable marker sequence selected from the group consisting of the Glutamine Synthase (GS) sequence and the Dihydrofolate Reductase (DHFR) sequence.

11. The nucleic acid construct of claim 1, wherein the selectable marker sequence is an antibiotic resistance marker sequence selected from the group consisting of neomycin resistance gene (neo), hygromycin B phosphotransferase gene and puromycin N-acetyl transferase gene sequences.

12. The nucleic acid construct of claim 1, wherein the second promoter sequence is selected from the group consisting of SV40, E. coli lac, E. coli trp, phage lambda PL, phage lambda PR, T3, T7, cytomegalovirus (CMV) immediate early, herpes simplex virus (HSV) thymidine kinase, alpha-lactalbumin, human elongation factor 1 alpha (hEF1alpha), and mouse metallothionein-I promoter sequences.

13. The nucleic acid construct of claim 1, wherein the nucleic acid sequence encoding a protein of interest encodes a protein selected from the group consisting of heavy and light chain immunoglobulin sequences.

14. The nucleic acid construct of any one of claim 1, wherein the insertion element is selected from the group consisting of a transposon insertion element, a recombinase insertion element, and a HDR insertion element.

15. The nucleic acid construct of claim 14, wherein the transposon insertion element is an inverted terminal repeat.

16. The nucleic acid construct of claim 15, wherein the construct comprises two inverted terminal repeats positioned 5′ to the first promoter and 3′ to the poly A signal sequence.

17. The nucleic acid construct of claim 14, wherein the recombinase insertion element is an attachment site (att).

18. The nucleic acid construct of claim 17, wherein the attachment site (att) is attB.

19-44. (canceled)

45. A host cell comprising the nucleic acid construct of claim 1.

46-49. (canceled)

50. The host cell of claim 45, wherein the host cell comprises from about 1 to 1000 copies of the nucleic acid construct.

51-62. (canceled)

63. A process for producing a protein of interest comprising culturing host cells according to claim 45, and purifying the protein of interest from the host cell culture.

64-68. (canceled)

69. A system comprising:
a first nucleic acid construct according to claim 1; and
a second nucleic acid construct encoding an enzyme.

70. The system of claim 69, wherein the enzyme is selected from the group consisting of a transposase, an integrase, a recombinase, a nuclease and a nickase.

71-99. (canceled)


SEQUENCE LISTING (12 sequences)

SEQ ID NO: 1 - DNA, 7516 bp, Artificial sequence
  synthetic
tcaatattggccattagccatattattcattggttatatagcataaatcaatattggctattggccattgcatacgttgtatctatatcataatatgtacatttatattggctcatgtccaatatgaccgccatgttggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaacaactgcgatcgcccgccccgttgacgcaaatgggcggtaggcgtgtacggtgggaggtctatataagcagagctcgtttagtgaaccgtcagatcactagaagctttattgcggtagtttatcacagttaaattgctaacgcagtcagtgcttctgacacaacagtctcgaacttaagctgcagtgactctcttaaggtagccttgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccactcccagttcaattacagctcttaaaaattggatctccattcgccattcaggctgcgcaactgctgggaaggacgatcagagcgggcctcttcgctattacgccagctggcgaaagggacgtggcaagcaaggcgattaagttgagttacgccaggattttcccagtcacgacgttgtaaaacgacggccagagaattataatacgactcactatagggcgaattcggatccgccgccaccatggtgacctacgccggagcctacgacagacagagccgggagagagagaacagcagcgccgccagccccgccacccagagaagcgccaacgaggccaaggccgccgccctgcagagagagatcgagagggccggcggcagattcagatttgtgggccacttcagcgaggcccctggcaccagcgccttcggcaccgccgagagacccgagttcgagagaatcctgaacgagtgtagggccggcaggctgaacatgatcatcgtgtacgacgtgtcccggttcagcaggctgaaggtgatggacgccatccctatcgtgtccgagctgctggccctgggcgtgaccatcgtgtccacccaggaaggcgtctttagacagggcaacgtgatggacctgatccacctgatcatgaggctggacgccagccacaaggagagcagcctgaagagcgccaagatcctggacaccaagaacctgcagagggagctgggcggctatgtgggcggcaaggccccctacggcttcgagctggtgtccgagaccaaggagatcacccggaacggcaggatggtgaacgtggtgatcaacaagctggcccacagcaccacccccctgaccggccccttcgagtttgagcccgacgtgatcaggtggtggtggcgggagatcaagacccacaagcacctgcctttcaagcccggcagccaggccgccatccaccccggcagcatcaccggcctgtgtaagagaatggacgccgacgccgtgcccaccagaggcgagaccatcggcaagaaaaccgccagcagcgcctgggaccccgccaccgtgatgagaatcctgagggaccctaggatcgccggcttcgccgccgaggtgatctacaagaagaagcccgacggcacccccaccaccaagatcgagggctacagaatccagagagaccccatcaccctgagacctgtggagctggactgtggccctatcatcgagcctgccgagtggtacgagctgcaggcctggctggacggcagaggcagaggcaagggcctgagcagaggccaggccatcctgagcgccatggacaagctgtactgtgagtgtggcgccgtgatgaccagcaagagaggcgaggagagcatcaaggacagctaccggtgccggagaagaaaggtggtggaccccagcgcccctggccagcacgagggcacctgtaatgtgagcatggccgccctggacaagttcgtggccgagcggatcttcaacaagatccggcacgccgagggcgacgaggagaccctggccctgctgtgggaggccgccagaagattcggcaagctgaccgaggcccccgagaagagcggcgagagggccaacctggtggccgagagagccgacgccctgaacgccctggaggagctgtacgaggacagagccgccggagcctatgacggccctgtgggcaggaagcacttcagaaagcagcaggccgccctgaccctgagacagcagggcgccgaggaaagactggccgagctggaggccgccgaggcccctaagctgcccctggatcagtggttccccgaggatgccgacgccgaccccaccggccccaagtcctggtggggcagagccagcgtggacgacaagagggtgttcgtgggcctgttcgtggataagatcgtggtgaccaagagcaccaccggcaggggccagggcacccccatcgagaagagagccagcatcacctgggccaagcctcccaccgacgacgacgaggatgacgcccaggacggcaccgaggacgtggccgcccctaagaaaaagcggaaagtgtgactcgagacgcgtgatatctttcccgggggtaccgtcgactgcggccgcgaattccaagcttgagtattctatcgtgtcacctaaataacttggcgtaatcatggtcatatctgtttcctgtgtgaaattgttatccgctcacaattccacacaacatacgagccggaagcataaagtgtaaagcctggggtgcctaatgagtgagctaactcacattaattgcgttgcgcgatgcttccattttgtgagggttaatgcttcgagaagacatgataagatacattgatgagtttggacaaaccacaacaagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatccgataaggatcgattccggagcctgaatggcgaatggacgcgccctgtagcggcgcattaagcgcggcgggtgtggtggttacgcgcacgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttcgcctgtgtaccttctgaggcggaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagcttgattcttctgacacaacagtctcgaacttaaggctagagccaccatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgatggccgcaataaaatatctttattttcattacatctgtgtgttggttttttgtgtgcgtacgaagatccgcgtatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatggctcgacagatct

SEQ ID NO: 2 - DNA, 5043 bp, Artificial sequence
  synthetic
gaattaattcataccagatcaccgaaaactgtcctccaaatgtgtccccctcacactcccaaattcgcgggcttctgcctcttagaccactctaccctattccccacactcaccggagccaaagccgcggcccttccgtttctttgctgtccggccattagccatattattcattggttatatagcataaatcaatattggctattggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcaataaaagagcccacaacccctcactcggcgcgccagtcttccgatagactgcgtcgcccgggtacccgtattcccaataaagcctcttgctgtttgcatccgaatcgtggtctcgctgttccttgggagggtctcctctgagtgattgactacccacgacgggggtctttcatttgggggctcgtccgggatttggagacccctgcccagggaccaccgacccaccaccgggaggtaagctggccagcaacttatctgtgtctgtccgattgtctagtgtctatgtttgatgttatgcgcctgcgtctgtactagttagctaactagctctgtatctggcggacccgtggtggaactgacgagttctgaacacccggccgcaaccctgggagacgtcccagggactttgggggccgtttttgtggcccgacctgaggaagggagtcgatgtggaatccgaccccgtcaggatatgtggttctggtaggagacgagaacctaaaacagttcccgcctccgtctgaatttttgctttcggtttggaaccgaagccgcgcgtcttgtctgctgcagcgctgcagcatcgttctgtgttgtctctgtctgactgtgtttctgtatttgtctgaaaattagggccagactgttaccactcccttaagtttgaccttaggtcactggaaagatgtcgagcggatcgctcacaaccagtcggtagatgtcaagaagagacgttgggttaccttctgctctgcagaatggccaacctttaacgtcggatggccgcgagacggcacctttaaccgagacctcatcacccaggttaagatcaaggtcttttcacctggcccgcatggacacccagaccaggtcccctacatcgtgacctgggaagccttggcttttgacccccctccctgggtcaagccctttgtacaccctaagcctccgcctcctcttcctccatccgccccgtctctcccccttgaacctcctcgttcgaccccgcctcgatcctccctttatccagccctcactccttctctaggcgccggaattgacgcgcgcgtaggcctgcggccgcagtactgacggacacaccgaagccccggcggcaaccctcagcggatgccccggggcttcacgttttcccaggtcagaagcggttttcgggagtagtgccccaactggggtaacctttgagttctctcagttgggggcgtagggtcgccgacatgacacaaggggttgtgaccggggtggacacgtacgcgggtgcttacgaccgtcagtcgcgcgagcgcgatagtctcgagttcgacatcgataaaataaaagattttatttagtctccagaaaaaggggggaatgaaagaccccacctgtaggtttggcaagctagcttaagtaacgccattttgcaaggcatggaaaaatacataactgagaatagaaaagttcagatcaaggtcaggaacagatggaacagggtcgaccggtcgaccggtcgaccctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggggcgccagtcctccgattgactgagtcgcccgggtacccgtgtatccaataaaccctcttgcagttgcatccgacttgtggtctcgctgttccttgggagggtctcctctgagtgattgactacccgtcagcgggggtctttcatttgggggctcgtccgggatcgggagacccctgcccagggaccaccgacccaccaccgggaggtaagctggctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtatgcatgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaa

SEQ ID NO: 3 - DNA, 5638 bp, Artificial sequence
  synthetic
gaattaattcataccagatcaccgaaaactgtcctccaaatgtgtccccctcacactcccaaattcgcgggcttctgcctcttagaccactctaccctattccccacactcaccggagccaaagccgcggcccttccgtttctttgctgtccggccattagccatattattcattggttatatagcataaatcaatattggctattggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcaataaaagagcccacaacccctcactcggcgcgccagtcttccgatagactgcgtcgcccgggtacccgtattcccaataaagcctcttgctgtttgcatccgaatcgtggtctcgctgttccttgggagggtctcctctgagtgattgactacccacgacgggggtctttcatttgggggctcgtccgggatttggagacccctgcccagggaccaccgacccaccaccgggaggtaagctggccagcaacttatctgtgtctgtccgattgtctagtgtctatgtttgatgttatgcgcctgcgtctgtactagttagctaactagctctgtatctggcggacccgtggtggaactgacgagttctgaacacccggccgcaaccctgggagacgtcccagggactttgggggccgtttttgtggcccgacctgaggaagggagtcgatgtggaatccgaccccgtcaggatatgtggttctggtaggagacgagaacctaaaacagttcccgcctccgtctgaatttttgctttcggtttggaaccgaagccgcgcgtcttgtctgctgcagcgctgcagcatcgttctgtgttgtctctgtctgactgtgtttctgtatttgtctgaaaattagggccagactgttaccactcccttaagtttgaccttaggtcactggaaagatgtcgagcggatcgctcacaaccagtcggtagatgtcaagaagagacgttgggttaccttctgctctgcagaatggccaacctttaacgtcggatggccgcgagacggcacctttaaccgagacctcatcacccaggttaagatcaaggtcttttcacctggcccgcatggacacccagaccaggtcccctacatcgtgacctgggaagccttggcttttgacccccctccctgggtcaagccctttgtacaccctaagcctccgcctcctcttcctccatccgccccgtctctcccccttgaacctcctcgttcgaccccgcctcgatcctccctttatccagccctcactccttctctaggcgccggaattgacgcgcgcgtaggcctgcggccgcagtactgacggacacaccgaagccccggcggcaaccctcagcggatgccccggggcttcacgttttcccaggtcagaagcggttttcgggagtagtgccccaactggggtaacctttgagttctctcagttgggggcgtagggtcgccgacatgacacaaggggttgtgaccggggtggacacgtacgcgggtgcttacgaccgtcagtcgcgcgagcgcgatagtctcgagttcgacatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataaaataaaagattttatttagtctccagaaaaaggggggaatgaaagaccccacctgtaggtttggcaagctagcttaagtaacgccattttgcaaggcatggaaaaatacataactgagaatagaaaagttcagatcaaggtcaggaacagatggaacagggtcgaccggtcgaccggtcgaccctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggggcgccagtcctccgattgactgagtcgcccgggtacccgtgtatccaataaaccctcttgcagttgcatccgacttgtggtctcgctgttccttgggagggtctcctctgagtgattgactacccgtcagcgggggtctttcatttgggggctcgtccgggatcgggagacccctgcccagggaccaccgacccaccaccgggaggtaagctggctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtatgcatgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaa

SEQ ID NO: 4 - DNA, 8348 bp, Artificial sequence
  synthetic
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcatttaaatgaaagaccccacctgtaggtttggcaagctagcttaagtaacgccattttgcaaggcatggaaaaatacataactgagaatagaaaagttcagatcaaggtcaggaacagatggaacagggtcgaccggtcgaccggtcgaccctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggggcgccagtcttccgatagactgcgtcgcccgggtacccgtattcccaataaagcctcttgctgtttgcatccgaatcgtggtctcgctgttccttgggagggtctcctctgagtgattgactacccacgacgggggtctttcatttgggggctcgtccgggatttggagacccctgcccagggaccaccgacccaccaccgggaggtaagctggccagcaacttatctgtgtctgtccgattgtctagtgtctatgtttgatgttatgcgcctgcgtctgtactagttagctaactagctctgtatctggcggacccgtggtggaactgacgagttctgaacacccggccgcaaccctgggagacgtcccagggactttgggggccgtttttgtggcccgacctgaggaagggagtcgatgtggaatccgaccccgtcaggatatgtggttctggtaggagacgagaacctaaaacagttcccgcctccgtctgaatttttgctttcggtttggaaccgaagccgcgcgtcttgtctgctgcagcgctgcagcatcgttctgtgttgtctctgtctgactgtgtttctgtatttgtctgaaaattagggccagactgttaccactcccttaagtttgaccttaggtcactggaaagatgtcgagcggatcgctcacaaccagtcggtagatgtcaagaagagacgttgggttaccttctgctctgcagaatggccaacctttaacgtcggatggccgcgagacggcacctttaaccgagacctcatcacccaggttaagatcaaggtcttttcacctggcccgcatggacacccagaccaggtcccctacatcgtgacctgggaagccttggcttttgacccccctccctgggtcaagccctttgtacaccctaagcctccgcctcctcttcctccatccgccccgtctctcccccttgaacctcctcgttcgaccccgcctcgatcctccctttatccagccctcactccttctctaggcgccggaattgccttccaccatggccacctcagcaagttcccacttgaacaaaaacatcaagcaaatgtacttgtgcctgccccagggtgagaaagtccaagccatgtatatctgggttgatggtactggagaaggactgcgctgcaaaacccgcaccctggactgtgagcccaagtgtgtagaagagttacctgagtggaattttgatggctctagtacctttcagtctgagggctccaacagtgacatgtatctcagccctgttgccatgtttcgggaccccttccgcagagatcccaacaagctggtgttctgtgaagttttcaagtacaaccggaagcctgcagagaccaatttaaggcactcgtgtaaacggataatggacatggtgagcaaccagcacccctggtttggaatggaacaggagtatactctgatgggaacagatgggcacccttttggttggccttccaatggctttcctgggccccaaggtccgtattactgtggtgtgggcgcagacaaagcctatggcagggatatcgtggaggctcactaccgcgcctgcttgtatgctggggtcaagattacaggaacaaatgctgaggtcatgcctgcccagtgggagttccaaataggaccctgtgaaggaatccgcatgggagatcatctctgggtggcccgtttcatcttgcatcgagtatgtgaagactttggggtaatagcaacctttgaccccaagcccattcctgggaactggaatggtgcaggctgccataccaactttagcaccaaggccatgcgggaggagaatggtctgaagcacatcgaggaggccatcgagaaactaagcaagcggcaccggtaccacattcgagcctacgatcccaaggggggcctggacaatgcccgtcgtctgactgggttccacgaaacgtccaacatcaacgacttttctgctggtgtcgccaatcgcagtgccagcatccgcattccccggactgtcggccaggagaagaaaggttactttgaagaccgccgcccctctgccaactgtgacccctttgcagtgacagaagccatcgtccgcacatgccttctcaatgagactggcgacgagcccttccaatacaaaaactaaagatccctatggctattggccaggttcaatactatgtattggccctatgccatatagtattccatatatgggttttcctattgacgtagatagcccctcccaatgggcggtcccatataccatatatggggcttcctaataccgcccatagccactcccccattgacgtcaatggtctctatatatggtctttcctattgacgtcatatgggcggtcctattgacgtatatggcgcctcccccattgacgtcaattacggtaaatggcccgcctggctcaatgcccattgacgtcaataggaccacccaccattgacgtcaatgggatggctcattgcccattcatatccgttctcacgccccctattgacgtcaatgacggtaaatggcccacttggcagtacatcaatatctattaatagtaacttggcaagtacattactattggaagtacgccagggtacattggcagtactcccattgacgtcaatggcggtaaatggcccgcgatggctgccaagtacatccccattgacgtcaatggggaggggcaatgacgcaaatgggcgttccattgacgtaaatgggcggtaggcgtgcctaatgggaggtctatataagcaatgctcgtttagggaaccgccattctgcctggggacgtcggaggagctcgaaagcttctagacaattgccgccaccatgatgtcctttgtctctctgctcctggttggcatcctattccatgccacccaggccagtgatacaggtagacctttcgtagagatgtacagtgaaatccccgaaattatacacatgactgaaggaagggagctcgtcattccctgccgggttacgtcacctaacatcactgttactttaaaaaagtttccacttgacactttgatccctgatggaaaacgcataatctgggacagtagaaagggcttcatcatatcaaatgcaacgtacaaagaaatagggcttctgacctgtgaagcaacagtcaatgggcatttgtataagacaaactatctcacacatcgacaaaccaatacaatcatagatgtcgttctgagtccgtctcatggaattgaactatctgttggagaaaagcttgtcttaaattgtacagcaagaactgaactaaatgtggggattgacttcaactgggaatacccttcttcgaagcatcagcataagaaacttgtaaaccgagacctaaaaacccagtctgggagtgagatgaagaagtttttgagcaccttaactatagatggtgtaacccggagtgaccaaggattgtacacctgtgcagcatccagtgggctgatgaccaagaaaaacagcacatttgtcagggtccatgaaaaagacaaaactcacacatgcccaccgtgcccagcacctgaactcctggggggaccctcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccacgggaggagcagtacaacagcacatatcgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctcccgggaaatgatgagatctcgagttcgacatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgatgggggaggctaactgaaacacggaaggagacaataccggaaggaacccgcgctatgacggcaataaaaagacagaataaaacgcacgggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagcggtcgacgatgtaggtcacggtctcgaagccgcggtgcgggtgccagggcgtgcccttgggctccccgggcgcgtactccacctcacccatctggtccatcatgatgaacgggtcgaggtggcggtagttgatcccggcgaacgcgcggcgcaccgggaagccctcgccctcgaaaccgctgggcgcggtggtcacggtgagcacgggacgtgcgacggcgtcggcgggtgcggatacgcggggcagcgtcagcgggttctcgacggtcacggcgggcatgatttccactgtacgcgtagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 5 - DNA, 7011 bp, Artificial sequence
  synthetic
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgggatccccgtcgacgatgtaggtcacggtctcgaagccgcggtgcgggtgccagggcgtgcccttgggctccccgggcgcgtactccacctcacccatctggtccatcatgatgaacgggtcgaggtggcggtagttgatcccggcgaacgcgcggcgcaccgggaagccctcgccctcgaaaccgctgggcgcggtggtcacggtgagcacgggacgtgcgacggcgtcggcgggtgcggatacgcggggcagcgtcagcgggttctcgacggtcacggcgggcatgtcgacccttccaccatggccacctcagcaagttcccacttgaacaaaaacatcaagcaaatgtacttgtgcctgccccagggtgagaaagtccaagccatgtatatctgggttgatggtactggagaaggactgcgctgcaaaacccgcaccctggactgtgagcccaagtgtgtagaagagttacctgagtggaattttgatggctctagtacctttcagtctgagggctccaacagtgacatgtatctcagccctgttgccatgtttcgggaccccttccgcagagatcccaacaagctggtgttctgtgaagttttcaagtacaaccggaagcctgcagagaccaatttaaggcactcgtgtaaacggataatggacatggtgagcaaccagcacccctggtttggaatggaacaggagtatactctgatgggaacagatgggcacccttttggttggccttccaatggctttcctgggccccaaggtccgtattactgtggtgtgggcgcagacaaagcctatggcagggatatcgtggaggctcactaccgcgcctgcttgtatgctggggtcaagattacaggaacaaatgctgaggtcatgcctgcccagtgggagttccaaataggaccctgtgaaggaatccgcatgggagatcatctctgggtggcccgtttcatcttgcatcgagtatgtgaagactttggggtaatagcaacctttgaccccaagcccattcctgggaactggaatggtgcaggctgccataccaactttagcaccaaggccatgcgggaggagaatggtctgaagcacatcgaggaggccatcgagaaactaagcaagcggcaccggtaccacattcgagcctacgatcccaaggggggcctggacaatgcccgtcgtctgactgggttccacgaaacgtccaacatcaacgacttttctgctggtgtcgccaatcgcagtgccagcatccgcattccccggactgtcggccaggagaagaaaggttactttgaagaccgccgcccctctgccaactgtgacccctttgcagtgacagaagccatcgtccgcacatgccttctcaatgagactggcgacgagcccttccaatacaaaaactaaagatccctatggctattggccaggttcaatactatgtattggccctatgccatatagtattccatatatgggttttcctattgacgtagatagcccctcccaatgggcggtcccatataccatatatggggcttcctaataccgcccatagccactcccccattgacgtcaatggtctctatatatggtctttcctattgacgtcatatgggcggtcctattgacgtatatggcgcctcccccattgacgtcaattacggtaaatggcccgcctggctcaatgcccattgacgtcaataggaccacccaccattgacgtcaatgggatggctcattgcccattcatatccgttctcacgccccctattgacgtcaatgacggtaaatggcccacttggcagtacatcaatatctattaatagtaacttggcaagtacattactattggaagtacgccagggtacattggcagtactcccattgacgtcaatggcggtaaatggcccgcgatggctgccaagtacatccccattgacgtcaatggggaggggcaatgacgcaaatgggcgttccattgacgtaaatgggcggtaggcgtgcctaatgggaggtctatataagcaatgctcgtttagggaaccgccattctgcctggggacgtcggaggagctcgaaagcttctagacaattgccgccaccatgatgtcctttgtctctctgctcctggttggcatcctattccatgccacccaggccagtgatacaggtagacctttcgtagagatgtacagtgaaatccccgaaattatacacatgactgaaggaagggagctcgtcattccctgccgggttacgtcacctaacatcactgttactttaaaaaagtttccacttgacactttgatccctgatggaaaacgcataatctgggacagtagaaagggcttcatcatatcaaatgcaacgtacaaagaaatagggcttctgacctgtgaagcaacagtcaatgggcatttgtataagacaaactatctcacacatcgacaaaccaatacaatcatagatgtcgttctgagtccgtctcatggaattgaactatctgttggagaaaagcttgtcttaaattgtacagcaagaactgaactaaatgtggggattgacttcaactgggaatacccttcttcgaagcatcagcataagaaacttgtaaaccgagacctaaaaacccagtctgggagtgagatgaagaagtttttgagcaccttaactatagatggtgtaacccggagtgaccaaggattgtacacctgtgcagcatccagtgggctgatgaccaagaaaaacagcacatttgtcagggtccatgaaaaagacaaaactcacacatgcccaccgtgcccagcacctgaactcctggggggaccctcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccacgggaggagcagtacaacagcacatatcgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctcccgggaaatgatgagatctcgagttcgacatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgatgggggaggctaactgaaacacggaaggagacaataccggaaggaacccgcgctatgacggcaataaaaagacagaataaaacgcacgggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagccctagcagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 6 - DNA, 5680 bp, Artificial sequence
  synthetic
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgggatccccgtcgacgatgtaggtcacggtctcgaagccgcggtgcgggtgccagggcgtgcccttgggctccccgggcgcgtactccacctcacccatctggtccatcatgatgaacgggtcgaggtggcggtagttgatcccggcgaacgcgcggcgcaccgggaagccctcgccctcgaaaccgctgggcgcggtggtcacggtgagcacgggacgtgcgacggcgtcggcgggtgcggatacgcggggcagcgtcagcgggttctcgacggtcacggcgggcatgtcgacccttccaccatggccacctcagcaagttcccacttgaacaaaaacatcaagcaaatgtacttgtgcctgccccagggtgagaaagtccaagccatgtatatctgggttgatggtactggagaaggactgcgctgcaaaacccgcaccctggactgtgagcccaagtgtgtagaagagttacctgagtggaattttgatggctctagtacctttcagtctgagggctccaacagtgacatgtatctcagccctgttgccatgtttcgggaccccttccgcagagatcccaacaagctggtgttctgtgaagttttcaagtacaaccggaagcctgcagagaccaatttaaggcactcgtgtaaacggataatggacatggtgagcaaccagcacccctggtttggaatggaacaggagtatactctgatgggaacagatgggcacccttttggttggccttccaatggctttcctgggccccaaggtccgtattactgtggtgtgggcgcagacaaagcctatggcagggatatcgtggaggctcactaccgcgcctgcttgtatgctggggtcaagattacaggaacaaatgctgaggtcatgcctgcccagtgggagttccaaataggaccctgtgaaggaatccgcatgggagatcatctctgggtggcccgtttcatcttgcatcgagtatgtgaagactttggggtaatagcaacctttgaccccaagcccattcctgggaactggaatggtgcaggctgccataccaactttagcaccaaggccatgcgggaggagaatggtctgaagcacatcgaggaggccatcgagaaactaagcaagcggcaccggtaccacattcgagcctacgatcccaaggggggcctggacaatgcccgtcgtctgactgggttccacgaaacgtccaacatcaacgacttttctgctggtgtcgccaatcgcagtgccagcatccgcattccccggactgtcggccaggagaagaaaggttactttgaagaccgccgcccctctgccaactgtgacccctttgcagtgacagaagccatcgtccgcacatgccttctcaatgagactggcgacgagcccttccaatacaaaaactaaagatccctatggctattggccaggttcaatactatgtattggccctatgccatatagtattccatatatgggttttcctattgacgtagatagcccctcccaatgggcggtcccatataccatatatggggcttcctaataccgcccatagccactcccccattgacgtcaatggtctctatatatggtctttcctattgacgtcatatgggcggtcctattgacgtatatggcgcctcccccattgacgtcaattacggtaaatggcccgcctggctcaatgcccattgacgtcaataggaccacccaccattgacgtcaatgggatggctcattgcccattcatatccgttctcacgccccctattgacgtcaatgacggtaaatggcccacttggcagtacatcaatatctattaatagtaacttggcaagtacattactattggaagtacgccagggtacattggcagtactcccattgacgtcaatggcggtaaatggcccgcgatggctgccaagtacatccccattgacgtcaatggggaggggcaatgacgcaaatgggcgttccattgacgtaaatgggcggtaggcgtgcctaatgggaggtctatataagcaatgctcgtttagggaaccgccattctgcctggggacgtcggaggagctcgaagcggccgcaagcttcaattgaggcctcctaggttaattaagtttaaacagatctctcgagttcgacatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgatgggggaggctaactgaaacacggaaggagacaataccggaaggaacccgcgctatgacggcaataaaaagacagaataaaacgcacgggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagccctagcagcttggccgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 7 - DNA, 8085 bp, Artificial sequence
  synthetic
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgggatccccgtcgacgatgtaggtcacggtctcgaagccgcggtgcgggtgccagggcgtgcccttgggctccccgggcgcgtactccacctcacccatctggtccatcatgatgaacgggtcgaggtggcggtagttgatcccggcgaacgcgcggcgcaccgggaagccctcgccctcgaaaccgctgggcgcggtggtcacggtgagcacgggacgtgcgacggcgtcggcgggtgcggatacgcggggcagcgtcagcgggttctcgacggtcacggcgggcatgtcgacccttccaccatggccacctcagcaagttcccacttgaacaaaaacatcaagcaaatgtacttgtgcctgccccagggtgagaaagtccaagccatgtatatctgggttgatggtactggagaaggactgcgctgcaaaacccgcaccctggactgtgagcccaagtgtgtagaagagttacctgagtggaattttgatggctctagtacctttcagtctgagggctccaacagtgacatgtatctcagccctgttgccatgtttcgggaccccttccgcagagatcccaacaagctggtgttctgtgaagttttcaagtacaaccggaagcctgcagagaccaatttaaggcactcgtgtaaacggataatggacatggtgagcaaccagcacccctggtttggaatggaacaggagtatactctgatgggaacagatgggcacccttttggttggccttccaatggctttcctgggccccaaggtccgtattactgtggtgtgggcgcagacaaagcctatggcagggatatcgtggaggctcactaccgcgcctgcttgtatgctggggtcaagattacaggaacaaatgctgaggtcatgcctgcccagtgggagttccaaataggaccctgtgaaggaatccgcatgggagatcatctctgggtggcccgtttcatcttgcatcgagtatgtgaagactttggggtaatagcaacctttgaccccaagcccattcctgggaactggaatggtgcaggctgccataccaactttagcaccaaggccatgcgggaggagaatggtctgaagcacatcgaggaggccatcgagaaactaagcaagcggcaccggtaccacattcgagcctacgatcccaaggggggcctggacaatgcccgtcgtctgactgggttccacgaaacgtccaacatcaacgacttttctgctggtgtcgccaatcgcagtgccagcatccgcattccccggactgtcggccaggagaagaaaggttactttgaagaccgccgcccctctgccaactgtgacccctttgcagtgacagaagccatcgtccgcacatgccttctcaatgagactggcgacgagcccttccaatacaaaaactaaagatccctatggctattggccaggttcaatactatgtattggccctatgccatatagtattccatatatgggttttcctattgacgtagatagcccctcccaatgggcggtcccatataccatatatggggcttcctaataccgcccatagccactcccccattgacgtcaatggtctctatatatggtctttcctattgacgtcatatgggcggtcctattgacgtatatggcgcctcccccattgacgtcaattacggtaaatggcccgcctggctcaatgcccattgacgtcaataggaccacccaccattgacgtcaatgggatggctcattgcccattcatatccgttctcacgccccctattgacgtcaatgacggtaaatggcccacttggcagtacatcaatatctattaatagtaacttggcaagtacattactattggaagtacgccagggtacattggcagtactcccattgacgtcaatggcggtaaatggcccgcgatggctgccaagtacatccccattgacgtcaatggggaggggcaatgacgcaaatgggcgttccattgacgtaaatgggcggtaggcgtgcctaatgggaggtctatataagcaatgctcgtttagggaaccgccattctgcctggggacgtcggaggagctcgaagcggccgccctagggtttaaacagatctatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgattactaatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggaattcggacggtgactgcagtgaataataaaatgtgtgtttgtccgaaatacgcgttttgagatttctgtcgccgactaaattcatgtcgcgcgatagtggtgtttatcgccgatagagatggcgatattggaaaaatcgatatttgaaaatatggcatattgaaaatgtcgccgatgtgagtttctgtgtaactgatatcgccatttttccaaaagtgatttttgggcatacgcgatatctggcgatagcgcttatatcgtttacgggggatggcgatagacgactttggtgacttgggcgattctgtgtgtcgcaaatatcgcagtttcgatataggtgacagacgatatgaggctatatcgccgatagaggcgacatcaagctggcacatggccaatgcatatcgatctatacattgaatcaatattggccattagccatattattcattggttatatagcataaatcaatattggctattggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggccgggaacggtgcattggaacgcggattccccgtgccaagagtgacgtaagtaccgcctatagagtctataggcccacccccttggcttcttatgcatgctatactgtttttggcttggggtctatacacccccgcttcctcatgttataggtgatggtatagcttagcctataggtgtgggttattgaccattattgaccactcccctattggtgacgatactttccattactaatccataacatggctctttgccacaactctctttattggctatatgccaatacactgtccttcagagactgacacggactctgtatttttacaggatggggtctcatttattatttacaaattcacatatacaacaccaccgtccccagtgcccgcagtttttattaaacataacgtgggatctccacgcgaatctcgggtacgtgttccggacatgggctcttctccggtagcggcggagcttctacatccgagccctgctcccatgcctccagcgactcatggtcgctcggcagctccttgctcctaacagtggaggccagacttaggcacagcacgatgcccaccaccaccagtgtgccgcacaaggccgtggcggtagggtatgtgtctgaaaatgagctcggggagcgggcttgcaccgctgacgcatttggaagacttaaggcagcggcagaagaagatgcaggcagctgagttgttgtgttctgataagagtcagaggtaactcccgttgcggtgctgttaacggtggagggcagtgtagtctgagcagtactcgttgctgccgcgcgcgccaccagacataatagctgacagactaacagactgttcctttccatgggtcttttctgcagtcaccgtccttgacacgaagttcgaatcaggataagggcgaattccgacgtaggcctattcgaagtctacttaattaaaagctttctagagcctcgagcgatgggggaggctaactgaaacacggaaggagacaataccggaaggaacccgcgctatgacggcaataaaaagacagaataaaacgcacgggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagccctagcagcttggccgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 8 - DNA, 7683 bp, Artificial sequence
  synthetic
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgggatccccgtcgacgatgtaggtcacggtctcgaagccgcggtgcgggtgccagggcgtgcccttgggctccccgggcgcgtactccacctcacccatctggtccatcatgatgaacgggtcgaggtggcggtagttgatcccggcgaacgcgcggcgcaccgggaagccctcgccctcgaaaccgctgggcgcggtggtcacggtgagcacgggacgtgcgacggcgtcggcgggtgcggatacgcggggcagcgtcagcgggttctcgacggtcacggcgggcatgtcgacccttccaccatggccacctcagcaagttcccacttgaacaaaaacatcaagcaaatgtacttgtgcctgccccagggtgagaaagtccaagccatgtatatctgggttgatggtactggagaaggactgcgctgcaaaacccgcaccctggactgtgagcccaagtgtgtagaagagttacctgagtggaattttgatggctctagtacctttcagtctgagggctccaacagtgacatgtatctcagccctgttgccatgtttcgggaccccttccgcagagatcccaacaagctggtgttctgtgaagttttcaagtacaaccggaagcctgcagagaccaatttaaggcactcgtgtaaacggataatggacatggtgagcaaccagcacccctggtttggaatggaacaggagtatactctgatgggaacagatgggcacccttttggttggccttccaatggctttcctgggccccaaggtccgtattactgtggtgtgggcgcagacaaagcctatggcagggatatcgtggaggctcactaccgcgcctgcttgtatgctggggtcaagattacaggaacaaatgctgaggtcatgcctgcccagtgggagttccaaataggaccctgtgaaggaatccgcatgggagatcatctctgggtggcccgtttcatcttgcatcgagtatgtgaagactttggggtaatagcaacctttgaccccaagcccattcctgggaactggaatggtgcaggctgccataccaactttagcaccaaggccatgcgggaggagaatggtctgaagcacatcgaggaggccatcgagaaactaagcaagcggcaccggtaccacattcgagcctacgatcccaaggggggcctggacaatgcccgtcgtctgactgggttccacgaaacgtccaacatcaacgacttttctgctggtgtcgccaatcgcagtgccagcatccgcattccccggactgtcggccaggagaagaaaggttactttgaagaccgccgcccctctgccaactgtgacccctttgcagtgacagaagccatcgtccgcacatgccttctcaatgagactggcgacgagcccttccaatacaaaaactaaagatccctatggctattggccaggttcaatactatgtattggccctatgccatatagtattccatatatgggttttcctattgacgtagatagcccctcccaatgggcggtcccatataccatatatggggcttcctaataccgcccatagccactcccccattgacgtcaatggtctctatatatggtctttcctattgacgtcatatgggcggtcctattgacgtatatggcgcctcccccattgacgtcaattacggtaaatggcccgcctggctcaatgcccattgacgtcaataggaccacccaccattgacgtcaatgggatggctcattgcccattcatatccgttctcacgccccctattgacgtcaatgacggtaaatggcccacttggcagtacatcaatatctattaatagtaacttggcaagtacattactattggaagtacgccagggtacattggcagtactcccattgacgtcaatggcggtaaatggcccgcgatggctgccaagtacatccccattgacgtcaatggggaggggcaatgacgcaaatgggcgttccattgacgtaaatgggcggtaggcgtgcctaatgggaggtctatataagcaatgctcgtttagggaaccgccattctgcctggggacgtcggaggagctcgaagcggccgccctagggtttaaacagatctatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgattactaatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggaattcggacggtgactgcagtgaataataaaatgtgtgtttgtccgaaatacgcgttttgagatttctgtcgccgactaaattcatgtcgcgcgatagtggtgtttatcgccgatagagatggcgatattggaaaaatcgatatttgaaaatatggcatattgaaaatgtcgccgatgtgagtttctgtgtaactgatatcgccatttttccaaaagtgatttttgggcatacgcgatatctggcgatagcgcttatatcgtttacgggggatggcgatagacgactttggtgacttgggcgattctgtgtgtcgcaaatatcgcagtttcgatataggtgacagacgatatgaggctatatcgccgatagaggcgacatcaagctggcacatggccaatgcatatcgatctatacattgaatcaatattggccattagccatattattcattggttatatagcataaatcaatattggctattggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccggatccttaaccgtgaaagcttaggccttctagagcctcgagttcgacatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgatgggggaggctaactgaaacacggaaggagacaataccggaaggaacccgcgctatgacggcaataaaaagacagaataaaacgcacgggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagccctagcagcttggccgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 9 - DNA, 10196 bp, Artificial sequence
  synthetic
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgggatccccgtcgacgatgtaggtcacggtctcgaagccgcggtgcgggtgccagggcgtgcccttgggctccccgggcgcgtactccacctcacccatctggtccatcatgatgaacgggtcgaggtggcggtagttgatcccggcgaacgcgcggcgcaccgggaagccctcgccctcgaaaccgctgggcgcggtggtcacggtgagcacgggacgtgcgacggcgtcggcgggtgcggatacgcggggcagcgtcagcgggttctcgacggtcacggcgggcatgtcgacccttccaccatggccacctcagcaagttcccacttgaacaaaaacatcaagcaaatgtacttgtgcctgccccagggtgagaaagtccaagccatgtatatctgggttgatggtactggagaaggactgcgctgcaaaacccgcaccctggactgtgagcccaagtgtgtagaagagttacctgagtggaattttgatggctctagtacctttcagtctgagggctccaacagtgacatgtatctcagccctgttgccatgtttcgggaccccttccgcagagatcccaacaagctggtgttctgtgaagttttcaagtacaaccggaagcctgcagagaccaatttaaggcactcgtgtaaacggataatggacatggtgagcaaccagcacccctggtttggaatggaacaggagtatactctgatgggaacagatgggcacccttttggttggccttccaatggctttcctgggccccaaggtccgtattactgtggtgtgggcgcagacaaagcctatggcagggatatcgtggaggctcactaccgcgcctgcttgtatgctggggtcaagattacaggaacaaatgctgaggtcatgcctgcccagtgggagttccaaataggaccctgtgaaggaatccgcatgggagatcatctctgggtggcccgtttcatcttgcatcgagtatgtgaagactttggggtaatagcaacctttgaccccaagcccattcctgggaactggaatggtgcaggctgccataccaactttagcaccaaggccatgcgggaggagaatggtctgaagcacatcgaggaggccatcgagaaactaagcaagcggcaccggtaccacattcgagcctacgatcccaaggggggcctggacaatgcccgtcgtctgactgggttccacgaaacgtccaacatcaacgacttttctgctggtgtcgccaatcgcagtgccagcatccgcattccccggactgtcggccaggagaagaaaggttactttgaagaccgccgcccctctgccaactgtgacccctttgcagtgacagaagccatcgtccgcacatgccttctcaatgagactggcgacgagcccttccaatacaaaaactaaagatccctatggctattggccaggttcaatactatgtattggccctatgccatatagtattccatatatgggttttcctattgacgtagatagcccctcccaatgggcggtcccatataccatatatggggcttcctaataccgcccatagccactcccccattgacgtcaatggtctctatatatggtctttcctattgacgtcatatgggcggtcctattgacgtatatggcgcctcccccattgacgtcaattacggtaaatggcccgcctggctcaatgcccattgacgtcaataggaccacccaccattgacgtcaatgggatggctcattgcccattcatatccgttctcacgccccctattgacgtcaatgacggtaaatggcccacttggcagtacatcaatatctattaatagtaacttggcaagtacattactattggaagtacgccagggtacattggcagtactcccattgacgtcaatggcggtaaatggcccgcgatggctgccaagtacatccccattgacgtcaatggggaggggcaatgacgcaaatgggcgttccattgacgtaaatgggcggtaggcgtgcctaatgggaggtctatataagcaatgctcgtttagggaaccgccattctgcctggggacgtcggaggagctcgaagcggccgccgccaccatgatgtcctttgtctctctgctcctggttggcatcctattccatgccacccaggcccaggtgcagctggtggagtccggcggcggcgtcgtgcagcccggccggtccctgcggctgtcctgcgccgcctccggcttcaccttctcctcctacaccatgcactgggtgcggcaggcccccggcaagggcctggagtgggtgactttcatctcctacgacggcaacaacaagtactacgccgactccgtgaagggccggttcaccatctcccgcgacaactccaagaacaccctgtacctgcagatgaactccctgcgggccgaggacaccgccatctactactgcgcccggaccggctggctgggccccttcgactactggggccagggcaccctggtgaccgtgtcctccgcctccaccaagggcccatcggtcttccccctggcaccctctagcaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagcgggttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcctccatcccgcgatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctcctgggaaatgatgagatctatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgattactaatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggaattcggacggtgactgcagtgaataataaaatgtgtgtttgtccgaaatacgcgttttgagatttctgtcgccgactaaattcatgtcgcgcgatagtggtgtttatcgccgatagagatggcgatattggaaaaatcgatatttgaaaatatggcatattgaaaatgtcgccgatgtgagtttctgtgtaactgatatcgccatttttccaaaagtgatttttgggcatacgcgatatctggcgatagcgcttatatcgtttacgggggatggcgatagacgactttggtgacttgggcgattctgtgtgtcgcaaatatcgcagtttcgatataggtgacagacgatatgaggctatatcgccgatagaggcgacatcaagctggcacatggccaatgcatatcgatctatacattgaatcaatattggccattagccatattattcattggttatatagcataaatcaatattggctattggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggccgggaacggtgcattggaacgcggattccccgtgccaagagtgacgtaagtaccgcctatagagtctataggcccacccccttggcttcttatgcatgctatactgtttttggcttggggtctatacacccccgcttcctcatgttataggtgatggtatagcttagcctataggtgtgggttattgaccattattgaccactcccctattggtgacgatactttccattactaatccataacatggctctttgccacaactctctttattggctatatgccaatacactgtccttcagagactgacacggactctgtatttttacaggatggggtctcatttattatttacaaattcacatatacaacaccaccgtccccagtgcccgcagtttttattaaacataacgtgggatctccacgcgaatctcgggtacgtgttccggacatgggctcttctccggtagcggcggagcttctacatccgagccctgctcccatgcctccagcgactcatggtcgctcggcagctccttgctcctaacagtggaggccagacttaggcacagcacgatgcccaccaccaccagtgtgccgcacaaggccgtggcggtagggtatgtgtctgaaaatgagctcggggagcgggcttgcaccgctgacgcatttggaagacttaaggcagcggcagaagaagatgcaggcagctgagttgttgtgttctgataagagtcagaggtaactcccgttgcggtgctgttaacggtggagggcagtgtagtctgagcagtactcgttgctgccgcgcgcgccaccagacataatagctgacagactaacagactgttcctttccatgggtcttttctgcagtcaccgtccttgacacgaagttcgaatcaggataagggcgaattccgacgtaggcctattcgaagtctacttaattaaaagcttgccgccaccatgatgtcctttgtctctctgctcctggttggcatcctattccatgccacccaggccgagatcgtgctgacccagtcccccggcaccctgtccctgtcccccggcgagcgggccaccctgtcctgccgggcctcccagtccgtgggctcctcctacctggcctggtaccagcagaagcccggccaggccccccggctgctgatctacggcgccttctcccgcgccaccggcatccccgaccggttctccggctccggctccggcaccgacttcaccctgaccatctcccggctggagcccgaggacttcgccgtgtactactgccagcagtacggctcctccccctggaccttcggccagggcaccaaggtggagatcaagcgaactgtggctgcaccatctgtcttcatcttcccgccatctgatgagcagcttaagtccggaactgctagcgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttagtgagatctcgagcgatgggggaggctaactgaaacacggaaggagacaataccggaaggaacccgcgctatgacggcaataaaaagacagaataaaacgcacgggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagccctagcagcttggccgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 10 - DNA, 10196 bp, Artificial sequence
  synthetic
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgggatccccgtcgacgatgtaggtcacggtctcgaagccgcggtgcgggtgccagggcgtgcccttgggctccccgggcgcgtactccacctcacccatctggtccatcatgatgaacgggtcgaggtggcggtagttgatcccggcgaacgcgcggcgcaccgggaagccctcgccctcgaaaccgctgggcgcggtggtcacggtgagcacgggacgtgcgacggcgtcggcgggtgcggatacgcggggcagcgtcagcgggttctcgacggtcacggcgggcatgtcgacccttccaccatggccacctcagcaagttcccacttgaacaaaaacatcaagcaaatgtacttgtgcctgccccagggtgagaaagtccaagccatgtatatctgggttgatggtactggagaaggactgcgctgcaaaacccgcaccctggactgtgagcccaagtgtgtagaagagttacctgagtggaattttgatggctctagtacctttcagtctgagggctccaacagtgacatgtatctcagccctgttgccatgtttcgggaccccttccgcagagatcccaacaagctggtgttctgtgaagttttcaagtacaaccggaagcctgcagagaccaatttaaggcactcgtgtaaacggataatggacatggtgagcaaccagcacccctggtttggaatggaacaggagtatactctgatgggaacagatgggcacccttttggttggccttccaatggctttcctgggccccaaggtccgtattactgtggtgtgggcgcagacaaagcctatggcagggatatcgtggaggctcactaccgcgcctgcttgtatgctggggtcaagattacaggaacaaatgctgaggtcatgcctgcccagtgggagttccaaataggaccctgtgaaggaatccgcatgggagatcatctctgggtggcccgtttcatcttgcatcgagtatgtgaagactttggggtaatagcaacctttgaccccaagcccattcctgggaactggaatggtgcaggctgccataccaactttagcaccaaggccatgcgggaggagaatggtctgaagcacatcgaggaggccatcgagaaactaagcaagcggcaccggtaccacattcgagcctacgatcccaaggggggcctggacaatgcccgtcgtctgactgggttccacgaaacgtccaacatcaacgacttttctgctggtgtcgccaatcgcagtgccagcatccgcattccccggactgtcggccaggagaagaaaggttactttgaagaccgccgcccctctgccaactgtgacccctttgcagtgacagaagccatcgtccgcacatgccttctcaatgagactggcgacgagcccttccaatacaaaaactaaagatccctatggctattggccaggttcaatactatgtattggccctatgccatatagtattccatatatgggttttcctattgacgtagatagcccctcccaatgggcggtcccatataccatatatggggcttcctaataccgcccatagccactcccccattgacgtcaatggtctctatatatggtctttcctattgacgtcatatgggcggtcctattgacgtatatggcgcctcccccattgacgtcaattacggtaaatggcccgcctggctcaatgcccattgacgtcaataggaccacccaccattgacgtcaatgggatggctcattgcccattcatatccgttctcacgccccctattgacgtcaatgacggtaaatggcccacttggcagtacatcaatatctattaatagtaacttggcaagtacattactattggaagtacgccagggtacattggcagtactcccattgacgtcaatggcggtaaatggcccgcgatggctgccaagtacatccccattgacgtcaatggggaggggcaatgacgcaaatgggcgttccattgacgtaaatgggcggtaggcgtgcctaatgggaggtctatataagcaatgctcgtttagggaaccgccattctgcctggggacgtcggaggagctcgaagcggccgccgccaccatgatgtcctttgtctctctgctcctggttggcatcctattccatgccacccaggccgagatcgtgctgacccagtcccccggcaccctgtccctgtcccccggcgagcgggccaccctgtcctgccgggcctcccagtccgtgggctcctcctacctggcctggtaccagcagaagcccggccaggccccccggctgctgatctacggcgccttctcccgcgccaccggcatccccgaccggttctccggctccggctccggcaccgacttcaccctgaccatctcccggctggagcccgaggacttcgccgtgtactactgccagcagtacggctcctccccctggaccttcggccagggcaccaaggtggagatcaagcgaactgtggctgcaccatctgtcttcatcttcccgccatctgatgagcagcttaagtccggaactgctagcgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttagtgagatctatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgattactaatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggaattcggacggtgactgcagtgaataataaaatgtgtgtttgtccgaaatacgcgttttgagatttctgtcgccgactaaattcatgtcgcgcgatagtggtgtttatcgccgatagagatggcgatattggaaaaatcgatatttgaaaatatggcatattgaaaatgtcgccgatgtgagtttctgtgtaactgatatcgccatttttccaaaagtgatttttgggcatacgcgatatctggcgatagcgcttatatcgtttacgggggatggcgatagacgactttggtgacttgggcgattctgtgtgtcgcaaatatcgcagtttcgatataggtgacagacgatatgaggctatatcgccgatagaggcgacatcaagctggcacatggccaatgcatatcgatctatacattgaatcaatattggccattagccatattattcattggttatatagcataaatcaatattggctattggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggccgggaacggtgcattggaacgcggattccccgtgccaagagtgacgtaagtaccgcctatagagtctataggcccacccccttggcttcttatgcatgctatactgtttttggcttggggtctatacacccccgcttcctcatgttataggtgatggtatagcttagcctataggtgtgggttattgaccattattgaccactcccctattggtgacgatactttccattactaatccataacatggctctttgccacaactctctttattggctatatgccaatacactgtccttcagagactgacacggactctgtatttttacaggatggggtctcatttattatttacaaattcacatatacaacaccaccgtccccagtgcccgcagtttttattaaacataacgtgggatctccacgcgaatctcgggtacgtgttccggacatgggctcttctccggtagcggcggagcttctacatccgagccctgctcccatgcctccagcgactcatggtcgctcggcagctccttgctcctaacagtggaggccagacttaggcacagcacgatgcccaccaccaccagtgtgccgcacaaggccgtggcggtagggtatgtgtctgaaaatgagctcggggagcgggcttgcaccgctgacgcatttggaagacttaaggcagcggcagaagaagatgcaggcagctgagttgttgtgttctgataagagtcagaggtaactcccgttgcggtgctgttaacggtggagggcagtgtagtctgagcagtactcgttgctgccgcgcgcgccaccagacataatagctgacagactaacagactgttcctttccatgggtcttttctgcagtcaccgtccttgacacgaagttcgaatcaggataagggcgaattccgacgtaggcctattcgaagtctacttaattaaaagcttgccgccaccatgatgtcctttgtctctctgctcctggttggcatcctattccatgccacccaggcccaggtgcagctggtggagtccggcggcggcgtcgtgcagcccggccggtccctgcggctgtcctgcgccgcctccggcttcaccttctcctcctacaccatgcactgggtgcggcaggcccccggcaagggcctggagtgggtgactttcatctcctacgacggcaacaacaagtactacgccgactccgtgaagggccggttcaccatctcccgcgacaactccaagaacaccctgtacctgcagatgaactccctgcgggccgaggacaccgccatctactactgcgcccggaccggctggctgggccccttcgactactggggccagggcaccctggtgaccgtgtcctccgcctccaccaagggcccatcggtcttccccctggcaccctctagcaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagcgggttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcctccatcccgcgatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctcctgggaaatgatgagatctcgagcgatgggggaggctaactgaaacacggaaggagacaataccggaaggaacccgcgctatgacggcaataaaaagacagaataaaacgcacgggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagccctagcagcttggccgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 11 - DNA, 9778 bp, Artificial sequence
  synthetic
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgggatccccgtcgacgatgtaggtcacggtctcgaagccgcggtgcgggtgccagggcgtgcccttgggctccccgggcgcgtactccacctcacccatctggtccatcatgatgaacgggtcgaggtggcggtagttgatcccggcgaacgcgcggcgcaccgggaagccctcgccctcgaaaccgctgggcgcggtggtcacggtgagcacgggacgtgcgacggcgtcggcgggtgcggatacgcggggcagcgtcagcgggttctcgacggtcacggcgggcatgtcgacccttccaccatggccacctcagcaagttcccacttgaacaaaaacatcaagcaaatgtacttgtgcctgccccagggtgagaaagtccaagccatgtatatctgggttgatggtactggagaaggactgcgctgcaaaacccgcaccctggactgtgagcccaagtgtgtagaagagttacctgagtggaattttgatggctctagtacctttcagtctgagggctccaacagtgacatgtatctcagccctgttgccatgtttcgggaccccttccgcagagatcccaacaagctggtgttctgtgaagttttcaagtacaaccggaagcctgcagagaccaatttaaggcactcgtgtaaacggataatggacatggtgagcaaccagcacccctggtttggaatggaacaggagtatactctgatgggaacagatgggcacccttttggttggccttccaatggctttcctgggccccaaggtccgtattactgtggtgtgggcgcagacaaagcctatggcagggatatcgtggaggctcactaccgcgcctgcttgtatgctggggtcaagattacaggaacaaatgctgaggtcatgcctgcccagtgggagttccaaataggaccctgtgaaggaatccgcatgggagatcatctctgggtggcccgtttcatcttgcatcgagtatgtgaagactttggggtaatagcaacctttgaccccaagcccattcctgggaactggaatggtgcaggctgccataccaactttagcaccaaggccatgcgggaggagaatggtctgaagcacatcgaggaggccatcgagaaactaagcaagcggcaccggtaccacattcgagcctacgatcccaaggggggcctggacaatgcccgtcgtctgactgggttccacgaaacgtccaacatcaacgacttttctgctggtgtcgccaatcgcagtgccagcatccgcattccccggactgtcggccaggagaagaaaggttactttgaagaccgccgcccctctgccaactgtgacccctttgcagtgacagaagccatcgtccgcacatgccttctcaatgagactggcgacgagcccttccaatacaaaaactaaagatccctatggctattggccaggttcaatactatgtattggccctatgccatatagtattccatatatgggttttcctattgacgtagatagcccctcccaatgggcggtcccatataccatatatggggcttcctaataccgcccatagccactcccccattgacgtcaatggtctctatatatggtctttcctattgacgtcatatgggcggtcctattgacgtatatggcgcctcccccattgacgtcaattacggtaaatggcccgcctggctcaatgcccattgacgtcaataggaccacccaccattgacgtcaatgggatggctcattgcccattcatatccgttctcacgccccctattgacgtcaatgacggtaaatggcccacttggcagtacatcaatatctattaatagtaacttggcaagtacattactattggaagtacgccagggtacattggcagtactcccattgacgtcaatggcggtaaatggcccgcgatggctgccaagtacatccccattgacgtcaatggggaggggcaatgacgcaaatgggcgttccattgacgtaaatgggcggtaggcgtgcctaatgggaggtctatataagcaatgctcgtttagggaaccgccattctgcctggggacgtcggaggagctcgaagcggccgccgccaccatgatgtcctttgtctctctgctcctggttggcatcctattccatgccacccaggcccaggtgcagctggtggagtccggcggcggcgtcgtgcagcccggccggtccctgcggctgtcctgcgccgcctccggcttcaccttctcctcctacaccatgcactgggtgcggcaggcccccggcaagggcctggagtgggtgactttcatctcctacgacggcaacaacaagtactacgccgactccgtgaagggccggttcaccatctcccgcgacaactccaagaacaccctgtacctgcagatgaactccctgcgggccgaggacaccgccatctactactgcgcccggaccggctggctgggccccttcgactactggggccagggcaccctggtgaccgtgtcctccgcctccaccaagggcccatcggtcttccccctggcaccctctagcaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagcgggttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcctccatcccgcgatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctcctgggaaatgatgagatctatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgattactaatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggaattcggacggtgactgcagtgaataataaaatgtgtgtttgtccgaaatacgcgttttgagatttctgtcgccgactaaattcatgtcgcgcgatagtggtgtttatcgccgatagagatggcgatattggaaaaatcgatatttgaaaatatggcatattgaaaatgtcgccgatgtgagtttctgtgtaactgatatcgccatttttccaaaagtgatttttgggcatacgcgatatctggcgatagcgcttatatcgtttacgggggatggcgatagacgactttggtgacttgggcgattctgtgtgtcgcaaatatcgcagtttcgatataggtgacagacgatatgaggctatatcgccgatagaggcgacatcaagctggcacatggccaatgcatatcgatctatacattgaatcaatattggccattagccatattattcattggttatatagcataaatcaatattggctattggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccggatccttaaccgtgaaagcttgccgccaccatgatgtcctttgtctctctgctcctggttggcatcctattccatgccacccaggccgagatcgtgctgacccagtcccccggcaccctgtccctgtcccccggcgagcgggccaccctgtcctgccgggcctcccagtccgtgggctcctcctacctggcctggtaccagcagaagcccggccaggccccccggctgctgatctacggcgccttctcccgcgccaccggcatccccgaccggttctccggctccggctccggcaccgacttcaccctgaccatctcccggctggagcccgaggacttcgccgtgtactactgccagcagtacggctcctccccctggaccttcggccagggcaccaaggtggagatcaagcgaactgtggctgcaccatctgtcttcatcttcccgccatctgatgagcagcttaagtccggaactgctagcgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttagtgagatctcgagttcgacatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgatgggggaggctaactgaaacacggaaggagacaataccggaaggaacccgcgctatgacggcaataaaaagacagaataaaacgcacgggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagccctagcagcttggccgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgacaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 12 - DNA, 9788 bp, Artificial sequence
  synthetic
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgggatccccgtcgacgatgtaggtcacggtctcgaagccgcggtgcgggtgccagggcgtgcccttgggctccccgggcgcgtactccacctcacccatctggtccatcatgatgaacgggtcgaggtggcggtagttgatcccggcgaacgcgcggcgcaccgggaagccctcgccctcgaaaccgctgggcgcggtggtcacggtgagcacgggacgtgcgacggcgtcggcgggtgcggatacgcggggcagcgtcagcgggttctcgacggtcacggcgggcatgtcgacccttccaccatggccacctcagcaagttcccacttgaacaaaaacatcaagcaaatgtacttgtgcctgccccagggtgagaaagtccaagccatgtatatctgggttgatggtactggagaaggactgcgctgcaaaacccgcaccctggactgtgagcccaagtgtgtagaagagttacctgagtggaattttgatggctctagtacctttcagtctgagggctccaacagtgacatgtatctcagccctgttgccatgtttcgggaccccttccgcagagatcccaacaagctggtgttctgtgaagttttcaagtacaaccggaagcctgcagagaccaatttaaggcactcgtgtaaacggataatggacatggtgagcaaccagcacccctggtttggaatggaacaggagtatactctgatgggaacagatgggcacccttttggttggccttccaatggctttcctgggccccaaggtccgtattactgtggtgtgggcgcagacaaagcctatggcagggatatcgtggaggctcactaccgcgcctgcttgtatgctggggtcaagattacaggaacaaatgctgaggtcatgcctgcccagtgggagttccaaataggaccctgtgaaggaatccgcatgggagatcatctctgggtggcccgtttcatcttgcatcgagtatgtgaagactttggggtaatagcaacctttgaccccaagcccattcctgggaactggaatggtgcaggctgccataccaactttagcaccaaggccatgcgggaggagaatggtctgaagcacatcgaggaggccatcgagaaactaagcaagcggcaccggtaccacattcgagcctacgatcccaaggggggcctggacaatgcccgtcgtctgactgggttccacgaaacgtccaacatcaacgacttttctgctggtgtcgccaatcgcagtgccagcatccgcattccccggactgtcggccaggagaagaaaggttactttgaagaccgccgcccctctgccaactgtgacccctttgcagtgacagaagccatcgtccgcacatgccttctcaatgagactggcgacgagcccttccaatacaaaaactaaagatccctatggctattggccaggttcaatactatgtattggccctatgccatatagtattccatatatgggttttcctattgacgtagatagcccctcccaatgggcggtcccatataccatatatggggcttcctaataccgcccatagccactcccccattgacgtcaatggtctctatatatggtctttcctattgacgtcatatgggcggtcctattgacgtatatggcgcctcccccattgacgtcaattacggtaaatggcccgcctggctcaatgcccattgacgtcaataggaccacccaccattgacgtcaatgggatggctcattgcccattcatatccgttctcacgccccctattgacgtcaatgacggtaaatggcccacttggcagtacatcaatatctattaatagtaacttggcaagtacattactattggaagtacgccagggtacattggcagtactcccattgacgtcaatggcggtaaatggcccgcgatggctgccaagtacatccccattgacgtcaatggggaggggcaatgacgcaaatgggcgttccattgacgtaaatgggcggtaggcgtgcctaatgggaggtctatataagcaatgctcgtttagggaaccgccattctgcctggggacgtcggaggagctcgaagcggccgccgccaccatgatgtcctttgtctctctgctcctggttggcatcctattccatgccacccaggccgagatcgtgctgacccagtcccccggcaccctgtccctgtcccccggcgagcgggccaccctgtcctgccgggcctcccagtccgtgggctcctcctacctggcctggtaccagcagaagcccggccaggccccccggctgctgatctacggcgccttctcccgcgccaccggcatccccgaccggttctccggctccggctccggcaccgacttcaccctgaccatctcccggctggagcccgaggacttcgccgtgtactactgccagcagtacggctcctccccctggaccttcggccagggcaccaaggtggagatcaagcgaactgtggctgcaccatctgtcttcatcttcccgccatctgatgagcagcttaagtccggaactgctagcgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttagtgagatctatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgattactaatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggaattcggacggtgactgcagtgaataataaaatgtgtgtttgtccgaaatacgcgttttgagatttctgtcgccgactaaattcatgtcgcgcgatagtggtgtttatcgccgatagagatggcgatattggaaaaatcgatatttgaaaatatggcatattgaaaatgtcgccgatgtgagtttctgtgtaactgatatcgccatttttccaaaagtgatttttgggcatacgcgatatctggcgatagcgcttatatcgtttacgggggatggcgatagacgactttggtgacttgggcgattctgtgtgtcgcaaatatcgcagtttcgatataggtgacagacgatatgaggctatatcgccgatagaggcgacatcaagctggcacatggccaatgcatatcgatctatacattgaatcaatattggccattagccatattattcattggttatatagcataaatcaatattggctattggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccggatccttaaccgtgaaagcttgccgccaccatgatgtcctttgtctctctgctcctggttggcatcctattccatgccacccaggcccaggtgcagctggtggagtccggcggcggcgtcgtgcagcccggccggtccctgcggctgtcctgcgccgcctccggcttcaccttctcctcctacaccatgcactgggtgcggcaggcccccggcaagggcctggagtgggtgactttcatctcctacgacggcaacaacaagtactacgccgactccgtgaagggccggttcaccatctcccgcgacaactccaagaacaccctgtacctgcagatgaactccctgcgggccgaggacaccgccatctactactgcgcccggaccggctggctgggccccttcgactactggggccagggcaccctggtgaccgtgtcctccgcctccaccaagggcccatcggtcttccccctggcaccctctagcaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagcgggttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcctccatcccgcgatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctcctgggaaatgatgagatctcgagttcgacatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgatgggggaggctaactgaaacacggaaggagacaataccggaaggaacccgcgctatgacggcaataaaaagacagaataaaacgcacgggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagccctagcagcttggccgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc